(19) United States
(12) Patent Application Publication
(10) Pub. No.: US 2026/0077005 A1
(43) Pub. Date: Mar. 19, 2026

(54) LENTIVIRAL VECTOR FORMULATIONS

(71) Applicants: BIOVERATIV THERAPEUTICS INC., Waltham, MA (US); FONDAZIONE TELETHON, Rome (IT); OSPEDALE SAN RAFFAELE S.R.L., Milan (IT)

(72) Inventors: Andrew Kroetsch, Waltham, MA (US); Isidro Zarraga, Waltham, MA (US)

(21) Appl. No.: 19/284,911

(22) Filed: Jul. 30, 2025

Related U.S. Application Data
(63) Continuation of application No. 17/038,031, filed on Sep. 30, 2020, now Pat. No. 12,403,164.
(60) Provisional application No. 62/908,390, filed on Sep. 30, 2019.

(51) Int. Cl. A61K 35/76; A61K 47/26; A61P 7/04; C12N 15/86

(52) U.S. Cl. CPC A61K 35/76; A61K 47/26; A61P 7/04; C12N 15/86

(57) ABSTRACT

Lentiviral vector (LV) formulations, and pharmaceutical compositions comprising such LV formulations, with improved stability and suitable for systemic administration are provided. Methods for treating disorders, especially blood disorders, using systemic administration of LV formulations are also provided.

### RELATED APPLICATIONS

This application is a continuation of U.S. patent application Ser. No. 17/038,031, filed Sep. 30, 2020, which claims priority to U.S. Provisional Patent Application Ser. No. 62/908,390, filed Sep. 30, 2019, the disclosures of which are hereby incorporated by reference in their entirety.

### SEQUENCE LISTING

The instant application contains a Sequence Listing which has been submitted electronically in XML format and is hereby incorporated by reference in its entirety. Said XML file, created on Jul. 29, 2025, is named 766511_SA9-472CON_ST26.xml and is 99,395 bytes in size.

### FIELD OF THE INVENTION

The present disclosure concerns formulations of recombinant lentiviral vectors (LVs) and related pharmaceutical products for use in the treatment of disease. In particular, it relates to formulations that improve LV stability and quality, while also being compatible for use in systemic and other types of administration to subjects for treating diseases, including bleeding disorders, such as hemophilia A and hemophilia B.

### BACKGROUND

Lentiviral vectors (LVs) and other viral vectors are an attractive tool for gene therapy (Thomas et al., 2003). LVs can transduce a broad range of tissues, including non-dividing cells such as hepatocytes, neurons and hematopoietic stem cells. Moreover, LVs can integrate into target cell genomes and provide long-term transgene expression.

An ongoing challenge in the field of gene therapy and vaccine development is to generate non-toxic liquid formulations that enable LVs to remain structurally stable and biologically active for longer periods of time and withstand conditions such as agitation, freeze/thawing, and storage at a range of temperatures. LV titer has been observed to decrease in a biphasic manner with increased freeze/thaw cycles and storage at higher temperatures (Kigashikawa and Chang 2001, Virology 280, 124-131). In order for gene therapy to be most effective, it is desirable to have lentiviral vectors that maintain their biological activity or potency.

The biological activity of an LV depends on the conformational integrity of an enclosed structure that consists of at least: (a) a core polynucleotide, (b) a shell of inter-linked capsid proteins surrounding the core polynucleotide, and (c) a glycoprotein-embedded lipid membrane surrounding the shell of inter-linked capsid proteins. Unlike organic and inorganic drugs, LVs are highly complex biological structures and minor chemical or physical stressors can contribute to the degradation of the structural integrity of the enclosed structure. Such stressors include osmolarity, buffer, pH, viscosity, electrolytes, agitation, and temperature fluctuations. The structural or conformational integrity of LVs is directly linked to their biological activity or potency. Thus, LVs may lose potency as a result of physical instabilities, including denaturation, soluble and insoluble aggregation, precipitation and adsorption, as well as chemical instabilities, including hydrolysis, deamidation, and oxidation. Any of these types of degradation can result in lowered biological activity, and can also potentially result in the formation of by-products or derivatives having increased toxicity and/or altered immunogenicity. A good formulation of LVs is thus crucially important to ensure not only a reasonable shelf-life, but also lowered toxicity upon administration to a subject, such as via systemic administration. Finding vehicles that stabilize LVs to result in robust formulations, in which the LVs are stable over a wide range of conditions, requires meticulous optimization of buffer type, pH, and excipients. For each set of conditions tested, the stability of the LVs needs to be measured via different experimental methods. Thus, in view of all of the factors that can be varied, finding optimal conditions for formulating LVs is challenging, and the composition of a good formulation is a priori unpredictable.

Accordingly, there is a need in the art to prepare formulations that are suitable for administration to a subject and that improve LV stability by preserving the quantity, structural integrity, and potency of the LVs under a range of conditions. Herein, we disclose formulations that demonstrate improved stability of LVs under a variety of conditions and that are suitable for systemic administration to subjects.

### SUMMARY

The present disclosure is based on the unexpected finding that lentiviral vector (LV) formulations with improved stability can be achieved when the LVs are suspended in a vehicle comprising a TRIS-free buffer system (e.g., a phosphate or histidine buffer) in combination with a carbohydrate (e.g., sucrose), a surfactant (e.g., poloxamer or polysorbate), and a salt (e.g., NaCl or other chloride salt). The contribution of a surfactant, e.g., poloxamer, to LV stability was surprising because surfactants are known in the art to destabilize particles bound by lipid membranes. Also surprising was the observation that an LV formulation at a pH range of from about 6.0 to about 7.5 (e.g., a pH of 6.5), improved LV stability, instead of destabilizing LV surface proteins (e.g., capsid proteins and VSV-G proteins) and promoting LV disassembly or breakdown. Moreover, the instant disclosure demonstrates that the LV formulations of the disclosure are particularly suitable for systemic administration (e.g., intravenous administration) to a subject.

In one aspect, the present disclosure provides a recombinant lentiviral vector preparation comprising: (a) a therapeutically effective dose of a recombinant lentiviral vector; (b) a TRIS-free buffer system; (c) a salt; (d) a surfactant; and (e) a carbohydrate, wherein the pharmaceutical composition is suitable for systemic administration to a human patient.

In certain embodiments, the lentiviral vector comprises a nucleotide sequence encoding VSV-G or a fragment thereof.

In certain embodiments, the buffer system comprises a phosphate buffer.

In certain embodiments, the concentration of the phosphate buffer is between 5 mM and 30 mM.

In certain embodiments, the concentration of the phosphate buffer is about 10 to about 20 mM, about 10 to about 15 mM, about 20 to about 30 mM, about 20 to about 25 mM, or about 15 to about 20 mM.

In certain embodiments, the concentration of the salt is between 80 mM and 150 mM.

In certain embodiments, the concentration of the salt is about 100 mM, about 110 mM, about 130 mM, or about 150 mM.

In certain embodiments, the salt is a chloride salt.

In certain embodiments, the chloride salt is NaCl

In certain embodiments, the surfactant is a poloxamer.

In certain embodiments, the poloxamer is selected from the group consisting of poloxamer 101 (P101), poloxamer 105 (P105), poloxamer 108 (P108), poloxamer 122 (P122), poloxamer 123 (P123), poloxamer 124 (P124), poloxamer 181 (P181), poloxamer 182 (P182), poloxamer 183 (P183), poloxamer 184 (P184), poloxamer 185 (P185), poloxamer 188 (P188), poloxamer 212 (P212), poloxamer 215 (P215), poloxamer 217 (P217), poloxamer 231 (P231), poloxamer 234 (P234), poloxamer 235 (P235), poloxamer 237 (P237), poloxamer 238 (P238), poloxamer 282 (P282), poloxamer 284 (P284), poloxamer 288 (P288), poloxamer 331 (P331), poloxamer 333 (P333), poloxamer 334 (P334), poloxamer 335 (P335), poloxamer 338 (P338), poloxamer 401 (P401), poloxamer 402 (P402), poloxamer 403 (P403), poloxamer 407 (P407), and a combination thereof.

In certain embodiments, the poloxamer is poloxamer 188 (P188).

In certain embodiments, the poloxamer is poloxamer 407 (P407).

In certain embodiments, surfactant is a polysorbate.

In certain embodiments, the polysorbate is selected from the group consisting of polysorbate 20, polysorbate 40, polysorbate 60, polysorbate 80, and a combination thereof.

In certain embodiments, the concentration of the surfactant is between 0.01% (w/v) and 0.1% (w/v).

In certain embodiments, the concentration of surfactant is about 0.03% (w/v), about 0.05% (w/v), about 0.07% (w/v), or about 0.09% (w/v).

In certain embodiments, the concentration of the carbohydrate is between 0.5% (w/v) and 5% (w/v).

In certain embodiments, the concentration of the carbohydrate is about 1% (w/v), about 2% (w/v), about 3% (w/v), or about 4% (w/v).

In certain embodiments, the carbohydrate is sucrose.

In certain embodiments, the pH of the buffer system or of the preparation is between 6.0 and 8.0.

In certain embodiments, the pH is between 6.0 and 7.0.

In certain embodiments, the pH is about 6.5.

In certain embodiments, the pH is about 7.0 to about 8.0.

In certain embodiments, the pH is about 7.3.

In one aspect, the present invention is directed to a recombinant lentiviral vector preparation comprising: (a) a therapeutically effective dose of a recombinant lentiviral vector; (b) a histidine buffer system; (c) a salt; (d) a surfactant; and (e) a carbohydrate, wherein the pharmaceutical composition is suitable for systemic administration to a human patient.

In certain embodiments, the lentiviral vector comprises a nucleotide sequence encoding VSV-G or a fragment thereof.

In certain embodiments, the concentration of the histidine buffer is between 5 mM and 30 mM.

In certain embodiments, the concentration of the histidine buffer is about 10 to about 20 mM, about 10 to about 15 mM, about 20 to about 30 mM, about 20 to about 25 mM, or about 15 to about 20 mM.

In certain embodiments, the concentration of salt is between 80 mM and 150 mM.

In certain embodiments, the concentration of the salt is about 100 mM, about 110 mM, about 130 mM, or about 150 mM.

In certain embodiments, the salt is a chloride salt.

In certain embodiments, the chloride salt is NaCl

In certain embodiments, the surfactant is a poloxamer.

In certain embodiments, the poloxamer is selected from the group consisting of poloxamer 101 (P101), poloxamer 105 (P105), poloxamer 108 (P108), poloxamer 122 (P122), poloxamer 123 (P123), poloxamer 124 (P124), poloxamer 181 (P181), poloxamer 182 (P182), poloxamer 183 (P183), poloxamer 184 (P184), poloxamer 185 (P185), poloxamer 188 (P188), poloxamer 212 (P212), poloxamer 215 (P215), poloxamer 217 (P217), poloxamer 231 (P231), poloxamer 234 (P234), poloxamer 235 (P235), poloxamer 237 (P237), poloxamer 238 (P238), poloxamer 282 (P282), poloxamer 284 (P284), poloxamer 288 (P288), poloxamer 331 (P331), poloxamer 333 (P333), poloxamer 334 (P334), poloxamer 335 (P335), poloxamer 338 (P338), poloxamer 401 (P401), poloxamer 402 (P402), poloxamer 403 (P403), poloxamer 407 (P407), and a combination thereof.

In certain embodiments, the poloxamer is poloxamer 188 (P188).

In certain embodiments, the poloxamer is poloxamer 407 (P407).

In certain embodiments, surfactant is a polysorbate.

In certain embodiments, the polysorbate is selected from the group consisting of polysorbate 20, polysorbate 40, polysorbate 60, polysorbate 80, and a combination thereof.

In certain embodiments, the concentration of the surfactant is between 0.01% (w/v) and 0.1% (w/v).

In certain embodiments, the concentration of surfactant is about 0.03% (w/v), about 0.05% (w/v), about 0.07% (w/v), or about 0.09% (w/v).

In certain embodiments, the concentration of the carbohydrate is between 0.5% (w/v) and 5% (w/v).

In certain embodiments, the concentration of the carbohydrate is about 1% (w/v), about 2% (w/v), about 3% (w/v), or about 4% (w/v).

In certain embodiments, the carbohydrate is sucrose.

In certain embodiments, the pH of the buffer system or of the preparation is between 6.0 and 8.0.

In certain embodiments, the pH is between 6.0 and 7.0.

In certain embodiments, the pH is about 6.5.

In certain embodiments, the pH is about 7.0 to about 8.0.

In certain embodiments, the pH is about 7.3.

In one aspect, the present invention is directed to a recombinant lentiviral vector preparation comprising: (a) a therapeutically effective dose of a recombinant lentiviral vector; (b) a phosphate buffer system; (c) a salt; (d) a surfactant; and (e) a carbohydrate, wherein the pharmaceutical composition is suitable for systemic administration to a human patient.

In certain embodiments, the lentiviral vector comprises a nucleotide sequence encoding VSV-G or a fragment thereof.

In certain embodiments, the concentration of the phosphate buffer is between 5 mM and 30 mM.

In certain embodiments, the concentration of the phosphate buffer is about 10 to about 20 mM, about 10 to about 15 mM, about 20 to about 30 mM, about 20 to about 25 mM, or about 15 to about 20 mM.

In certain embodiments, the concentration of the salt is between 80 mM and 150 mM.

In certain embodiments, the concentration of the salt is about 100 mM, about 110 mM, about 130 mM, or about 150 mM.

In certain embodiments, the salt is a chloride salt.

In certain embodiments, the chloride salt is NaCl

In certain embodiments, the surfactant is a poloxamer.

In certain embodiments, the poloxamer is selected from the group consisting of poloxamer 101 (P101), poloxamer 105 (P105), poloxamer 108 (P108), poloxamer 122 (P122), poloxamer 123 (P123), poloxamer 124 (P124), poloxamer 181 (P181), poloxamer 182 (P182), poloxamer 183 (P183), poloxamer 184 (P184), poloxamer 185 (P185), poloxamer 188 (P188), poloxamer 212 (P212), poloxamer 215 (P215), poloxamer 217 (P217), poloxamer 231 (P231), poloxamer 234 (P234), poloxamer 235 (P235), poloxamer 237 (P237), poloxamer 238 (P238), poloxamer 282 (P282), poloxamer 284 (P284), poloxamer 288 (P288), poloxamer 331 (P331), poloxamer 333 (P333), poloxamer 334 (P334), poloxamer 335 (P335), poloxamer 338 (P338), poloxamer 401 (P401), poloxamer 402 (P402), poloxamer 403 (P403), poloxamer 407 (P407), and a combination thereof.

In certain embodiments, the poloxamer is poloxamer 188 (P188).

In certain embodiments, the poloxamer is poloxamer 407 (P407).

In certain embodiments, surfactant is a polysorbate.

In certain embodiments, the polysorbate is selected from the group consisting of polysorbate 20, polysorbate 40, polysorbate 60, polysorbate 80, and a combination thereof.

In certain embodiments, the concentration of the surfactant is between 0.01% (w/v) and 0.1% (w/v).

In certain embodiments, the concentration of surfactant is about 0.03% (w/v), about 0.05% (w/v), about 0.07% (w/v), or about 0.09% (w/v).

In certain embodiments, the concentration of the carbohydrate is between 0.5% (w/v) and 5% (w/v).

In certain embodiments, the concentration of the carbohydrate is about 1% (w/v), about 2% (w/v), about 3% (w/v), or about 4% (w/v).

In certain embodiments, the carbohydrate is sucrose.

In certain embodiments, the pH of the buffer system or of the preparation is between 6.0 and 8.0.

In certain embodiments, the pH is between 6.0 and 7.0.

In certain embodiments, the pH is about 6.5.

In certain embodiments, the pH is about 7.0 to about 8.0.

In certain embodiments, the pH is about 7.3.

In certain embodiments, the recombinant lentiviral vector further comprises a nucleotide sequence at least 80% identical to the Factor VIII (FVIII) coding sequence set forth in SEQ ID NO: 1 or SEQ ID NO: 2.

In certain embodiments, the recombinant lentiviral vector further comprises the Factor VIII (FVIII) coding sequence set forth in SEQ ID NO: 1 or SEQ ID NO: 2.

In certain embodiments, the recombinant lentiviral vector further comprises a nucleotide sequence at least 80% identical to the Factor IX (FIX) coding sequence set forth in SEQ ID NO: 3.

In certain embodiments, the recombinant lentiviral vector further comprises the Factor IX (FIX) coding sequence set forth in SEQ ID NO: 3.

In certain embodiments, the recombinant lentiviral vector further comprises an enhanced transthyretin (ET) promoter.

In certain embodiments, the recombinant lentiviral vector further comprises a nucleotide sequence at least 90% identical to the target sequence for miR-142 set forth in SEQ ID NO: 7.

In certain embodiments, the recombinant lentiviral vector is isolated from transfected host cells selected from the group of: a CHO cell, a HEK293 cell, a BHK21 cell, a PER.C6 cell, an NSO cell, and a CAP cell.

In certain embodiments, the host cells are CD47-positive host cells.

In one aspect, the present invention is directed to a method of treating a human patient with a disorder, wherein the human patient is administered a recombinant lentiviral vector preparation comprising: (a) a therapeutically effective dose of a recombinant lentiviral vector; (b) a TRIS-free buffer system; (c) a salt; (d) a surfactant; and (e) a carbohydrate, wherein the pharmaceutical composition is suitable for systemic administration to a human patient.

In certain embodiments, the preparation is administered systemically to the human patient.

In certain embodiments, the preparation is administered intravenously.

In certain embodiments, the disorder is a bleeding disorder.

In certain embodiments, the bleeding disorder is hemophilia A or hemophilia B.

In another aspect, a recombinant lentiviral vector preparation comprising: (a) a therapeutically effective dose of a recombinant lentiviral vector; (b) a TRIS-free buffer system; (c) a salt; (d) a surfactant; and (e) a carbohydrate, wherein the pH of the buffer system or of the preparation is from about 6.0 to about 7.5, and wherein the pharmaceutical composition is suitable for systemic administration to a human patient, is provided.

In certain exemplary embodiments, the lentiviral vector comprises a nucleotide sequence encoding VSV-G or a fragment thereof.

In certain exemplary embodiments, the buffer system comprises a phosphate buffer or a histidine buffer. In certain exemplary embodiments, the concentration of the phosphate buffer is from about 5 mM to about 30 mM. In certain exemplary embodiments, the concentration of the phosphate buffer is from about 10 mM to about 20 mM, from about 10 mM to about 15 mM, from about 20 mM to about 30 mM, from about 20 mM to about 25 mM, or from about 15 mM to about 20 mM. In certain exemplary embodiments, the concentration of the histidine buffer is from about 5 mM to about 30 mM. In certain exemplary embodiments, the concentration of the histidine buffer is from about 10 mM to about 20 mM, from about 10 mM to about 15 mM, from about 20 mM to about 30 mM, from about 20 mM to about 25 mM, or from about 15 mM to about 20 mM.

In certain exemplary embodiments, the concentration of the salt is from about 80 mM to about 150 mM. In certain exemplary embodiments, the concentration of the salt is about 100 mM, about 110 mM, about 130 mM, or about 150 mM. In certain exemplary embodiments, the salt is a chloride salt. In certain exemplary embodiments, the chloride salt is NaCl.

In certain exemplary embodiments, the surfactant is a poloxamer. In certain exemplary embodiments, the poloxamer is selected from the group consisting of poloxamer 101 (P101), poloxamer 105 (P105), poloxamer 108 (P108), poloxamer 122 (P122), poloxamer 123 (P123), poloxamer 124 (P124), poloxamer 181 (P181), poloxamer 182 (P182), poloxamer 183 (P183), poloxamer 184 (P184), poloxamer 185 (P185), poloxamer 188 (P188), poloxamer 212 (P212), poloxamer 215 (P215), poloxamer 217 (P217), poloxamer 231 (P231), poloxamer 234 (P234), poloxamer 235 (P235), poloxamer 237 (P237), poloxamer 238 (P238), poloxamer 282 (P282), poloxamer 284 (P284), poloxamer 288 (P288), poloxamer 331 (P331), poloxamer 333 (P333), poloxamer 334 (P334), poloxamer 335 (P335), poloxamer 338 (P338), poloxamer 401 (P401), poloxamer 402 (P402), poloxamer 403 (P403), poloxamer 407 (P407), and a combination thereof. In certain exemplary embodiments, the poloxamer is poloxamer 188 (P188). In certain exemplary embodiments, the poloxamer is poloxamer 407 (P407).

In certain exemplary embodiments, the surfactant is a polysorbate. In certain exemplary embodiments, the polysorbate is selected from the group consisting of polysorbate 20, polysorbate 40, polysorbate 60, polysorbate 80, and a combination thereof. In certain exemplary embodiments, the concentration of the surfactant is from about 0.01% (w/v) to about 0.1% (w/v). In certain exemplary embodiments, the concentration of surfactant is about 0.03% (w/v), about 0.05% (w/v), about 0.07% (w/v), or about 0.09% (w/v).

In certain exemplary embodiments, the concentration of the carbohydrate is from about 0.5% (w/v) to about 5% (w/v). In certain exemplary embodiments, the concentration of the carbohydrate is about 1% (w/v), about 2% (w/v), about 3% (w/v), or about 4% (w/v). In certain exemplary embodiments, the carbohydrate is sucrose.

In certain exemplary embodiments, the preparation comprises: (a) a therapeutically effective dose of a recombinant lentiviral vector; (b) about 10 mM phosphate; (c) about 100 mM sodium chloride; (d) about 0.05% (w/v) poloxamer 188; and (e) about 3% (w/v) sucrose, wherein the pH of the preparation is about 7.3, and wherein the pharmaceutical composition is suitable for systemic administration to a human patient.

In certain exemplary embodiments, the preparation comprises: (a) a therapeutically effective dose of a recombinant lentiviral vector; (b) about 10 mM phosphate; (c) about 130 mM sodium chloride; (d) about 0.05% (w/v) poloxamer 188; and (e) about 1% (w/v) sucrose, wherein the pH of the preparation is about 7.3, and wherein the pharmaceutical composition is suitable for systemic administration to a human patient.

In certain exemplary embodiments, the preparation comprises: (a) a therapeutically effective dose of a recombinant lentiviral vector; (b) about 20 mM histidine; (c) about 100 mM sodium chloride; (d) about 0.05% (w/v) poloxamer 188; and (e) about 3% (w/v) sucrose, wherein the pH of the preparation is about 6.5, and wherein the pharmaceutical composition is suitable for systemic administration to a human patient.

In certain exemplary embodiments, the preparation comprises: (a) a therapeutically effective dose of a recombinant lentiviral vector; (b) about 10 mM phosphate; (c) about 100 mM sodium chloride; (d) about 0.05% (w/v) poloxamer 188; and (e) about 3% (w/v) sucrose, wherein the pH of the preparation is about 7.0, and wherein the pharmaceutical composition is suitable for systemic administration to a human patient.

In certain exemplary embodiments, the preparation comprises: (a) a therapeutically effective dose of a recombinant lentiviral vector; (b) about 20 mM histidine; (c) about 100 mM sodium chloride; (d) about 0.05% (w/v) poloxamer 188; and (e) about 3% (w/v) sucrose, wherein the pH of the preparation is about 7.0, and wherein the pharmaceutical composition is suitable for systemic administration to a human patient.

In certain exemplary embodiments, the recombinant lentiviral vector comprises a nucleic acid comprising a nucleotide sequence at least 80%, at least 85%, at least 90%, at least 95%, or at least 99% identical to a Factor VIII (FVIII) coding sequence set forth in SEQ ID NO: 1 or SEQ ID NO: 2. In certain exemplary embodiments, the recombinant lentiviral vector comprises a nucleic acid comprising a Factor VIII (FVIII) coding sequence set forth in SEQ ID NO: 1 or SEQ ID NO: 2. In certain exemplary embodiments, the recombinant lentiviral vector comprises a nucleic acid consisting of a Factor VIII (FVIII) coding sequence set forth in SEQ ID NO: 1 or SEQ ID NO: 2.

In certain exemplary embodiments, the recombinant lentiviral vector comprises a nucleic acid comprising a nucleotide sequence at least 80%, at least 85%, at least 90%, at least 95%, or at least 99% identical to a Factor IX (FIX) coding sequence set forth in SEQ ID NO: 3. In certain exemplary embodiments, the recombinant lentiviral vector comprises a nucleic acid comprising the Factor IX (FIX) coding sequence set forth in SEQ ID NO: 3. In certain exemplary embodiments, the recombinant lentiviral vector comprises a nucleic acid consisting of the Factor IX (FIX) coding sequence set forth in SEQ ID NO: 3.

In certain exemplary embodiments, the recombinant lentiviral vector comprises an enhanced transthyretin (ET) promoter.

In certain exemplary embodiments, the recombinant lentiviral vector further comprises a nucleotide sequence at least 90% identical to the target sequence for miR-142 set forth in SEQ ID NO: 7.

In certain exemplary embodiments, the recombinant lentiviral vector is isolated from a transfected host cell selected from the group of: a CHO cell, a HEK293 cell, a BHK21 cell, a PER.C6 cell, an NSO cell, and a CAP cell. In certain exemplary embodiments, the host cell is a CD47-positive host cell.

In another aspect, a method of treating a human patient with a disorder, comprising administering to the human patient a recombinant lentiviral vector preparation described herein, is provided.

In certain exemplary embodiments, the preparation is administered systemically to the human patient. In certain exemplary embodiments, the preparation is administered intravenously.

In certain exemplary embodiments, the disorder is a bleeding disorder. In certain exemplary embodiments, the bleeding disorder is hemophilia A or hemophilia B.

### BRIEF DESCRIPTION OF THE DRAWINGS

The foregoing and other features and advantages of the present invention will be more fully understood from the following detailed description of illustrative embodiments taken in conjunction with the accompanying drawings. The patent or application file contains at least one drawing executed in color. Copies of this patent or patent application publication with color drawing(s) will be provided by the Office upon request and payment of the necessary fee.

FIG. 1 depicts characterization of lentiviral vector (LV) formulation upon processing into the vehicle Phosphate (10 mM Phosphate, 100 mM NaCl, 3% (w/v) sucrose, 0.05% (w/v) P188, pH 7.3). The drug substance (DS) pool shows a clean monomeric peak, which after ultrafiltration/diafiltration into the final vehicle buffer (post tangential flow filtration—TFF) shifts slightly to a larger size and there is presence of some larger particles. Without being bound to theory this may be due to physical degradation of the particle during the stress of processing the material. The final DP has been filtered through a 0.22 μm sized filter membrane and the profile comes back in line with the DS pool at the start of processing. The effects of the TFF stress can be visually seen in the pictures shown in FIGS. 2A-2B.

FIGS. 2A-2B depict effects of sterile filtration on lentiviral vectors (LVs) in the vehicle Phosphate (10 mM Phosphate, 100 mM NaCl, 3% (w/v) sucrose, 0.05% (w/v) P188, pH 7.3) post tangential flow filtration (TFF) (FIG. 2A) and in the final drug substance (DS) pool (FIG. 2B).

FIGS. 3A-3B depict stability of lentiviral vectors (LVs) in the vehicle TSSM (20 mM TRIS, 100 mM NaCl, 1% (w/v) Sucrose, 1% (w/v) Mannitol, pH 7.3), without the addition of 1% (w/v) P188 (FIG. 3A) and with the addition of 1% (w/v) P188 (FIG. 3B), as measured by p24 concentration using p24 ELISA.

FIG. 4 depicts stability of lentiviral vectors (LVs) in the vehicle TSSM (20 mM TRIS, 100 mM NaCl, 1% (w/v) Sucrose, 1% (w/v) Mannitol, pH 7.3) upon agitation, as measured by particle concentration and particle size using NanoSight. Stability study using TSSM formulation: 20 mM Tris, 100 mM NaCl, 1% (w/v) Sucrose, 1% (w/v) Mannitol, pH 7.3, as indicated with and without Poloxomer 188 (P188). Measurements were made using NanoSight.

FIGS. 5A-5B depict stability of lentiviral vectors (LVs) in the vehicle TSSM (20 mM TRIS, 100 mM NaCl, 1% (w/v) Sucrose, 1% (w/v) Mannitol, pH 7.3) upon agitation, as measured by particle concentration and particle size using NanoSight. FIG. 5A shows that upon agitation stress the lentiviral particles only seem to grow slightly in size. The main peak is assumed to be monomeric lentiviral vector (˜130 nm) and the smaller larger peaks may be degradation of the monomeric particle. FIG. 5B shows that the addition of 1% (w/v) poloxamer 188 (P188) did not interfere with the lentiviral particle.

FIGS. 6A-6B depict stability of lentiviral vectors (LVs) in the vehicle TSSM (20 mM TRIS, 100 mM NaCl, 1% (w/v) Sucrose, 1% (w/v) Mannitol, pH 7.3) at 37° C. over 0, 3, 7, and 14 days, as measured by functional titer in TU/ml (FIG. 6A) and % of T0 (FIG. 6B) using ddPCR. FIG. 6B is normalized to time 0. Each group of bars represent a dilution series of: undiluted (no dilution), 20 fold dilution (20×), 100 fold dilution (100×).

FIGS. 7A-7B depict stability of lentiviral vectors (LVs) in the vehicle TSSM (20 mM TRIS, 100 mM NaCl, 1% (w/v) Sucrose, 1% (w/v) Mannitol, pH 7.3) at 37° C. over 0, 3, 7, and 14 days, as measured by functional titer in TU/ml using ddPCR and particle concentration using NanoSight (FIG. 7A) or functional titer overlaid with p24 data (FIG. 7B).

FIGS. 8A-8B. FIG. 8A depict stability of lentiviral vectors (LVs) in the vehicle TSSM (20 mM TRIS, 100 mM NaCl, 1% (w/v) Sucrose, 1% (w/v) Mannitol, pH 7.3) at 37° C. as a function of incubation time in day and weeks, as measured by particle concentration and size using Nanosight. FIG. 8B is reporting the results of the 37° C. stability experiment on a log scale in order to more clearly see differences in particle size over time.

FIG. 9 depicts stability of lentiviral vectors (LVs) in the vehicle Phosphate Formulation (10 mM Phosphate, 100 mM NaCl, 3% (w/v) sucrose, 0.05% (w/v) P188, pH 7.3) as a function of incubation time, incubation temperature, agitation, and freeze/thaw (F/T) cycles in days, as measured by functional titer in % of T0 using ddPCR and p24 concentration using p24 ELISA.

FIGS. 10A-10B depict stability of lentiviral vectors (LVs) at 37° C. using Phosphate formulation (10 mM Phosphate, 100 mM NaCl, 3% (w/v) sucrose, 0.05% (w/v) P188, pH 7.3) as a function of time in days, as measured by particle concentration and particle size distribution using NanoSight. FIG. 10A is reporting the NanoSight data that is normalized to 1 in order to make differences in degradation peaks more visible. FIG. 10B presents the raw data showing a decrease in the monomeric peak over time at 37° C. incubation.

FIGS. 11A-11B depict stability of lentiviral vectors (LVs) comparing three formulations: Formulation 1. Phosphate formulation: 10 mM Phosphate, 100 mM NaCl, 3% (w/v) sucrose, 0.05% (w/v) P188, pH 7.3; Formulation 2. 10 mM Phosphate, 130 mM NaCl, 1% (w/v) sucrose, 0.05% (w/v) P188, pH 7.3; Formulation 3. 20 mM Histidine, 100 mM NaCl, 3% (w/v) sucrose, 0.05% (w/v) P188, pH 6.5; over 5 and 10 cycles of Freezing and Thawing (F/T) (FIG. 11A) and comparing 3 days at room temperature (RT) and RT with agitation (orbital shaker, 350 rpm) (FIG. 11B), as measured by functional titer using ddPCR.

FIGS. 12A-12B depict stability of lentiviral vectors (LVs) comparing three formulations): Formulation 1 (Phos). Phosphate formulation: 10 mM Phosphate, 100 mM NaCl, 3% (w/v) sucrose, 0.05% (w/v) P188, pH 7.3; Formulation 2 (Phos. higherSalt). 10 mM Phosphate, 130 mM NaCl, 1% (w/v) sucrose, 0.05% (w/v) P188, pH 7.3; Formulation 3 (Hist). 20 mM Histidine, 100 mM NaCl, 3% (w/v) sucrose, 0.05% (w/v) P188, pH 6.5, as measured by number of particles using NanoSight. FIG. 12A shows data 3 days at room temperature (RT) and RT with agitation (orbital shaker, 350 rpm), while FIG. 12B shows data for 5 and 10 cycles of Freezing and Thawing (F/T).

FIG. 13 depicts a mock in-use stability study with one formulation over 6 hours at room temperature exposed to an IV bag: Formulation 1 (Phosphate Buffer): 10 mM Phosphate, 100 mM NaCl, 3% (w/v) sucrose, 0.05% (w/v) P188, pH 7.3.

FIG. 14 depicts a formulation buffer stability study comparing container closure (Schott Type 1 glass vials and West CZ COP vials) performance over one freezing and thawing cycle (−80° C. overnight, thaw in 37° C. water bath): Formulation 1 (Phosphate Buffer): 10 mM Phosphate, 100 mM NaCl, 3% (w/v) sucrose, 0.05% (w/v) P188, pH 7.3. Particle concentration was obtained using Microflow Imaging (MFI).

FIG. 15 depicts a vial strain study using Schott Type 1 glass vials over one freezing and thawing cycle (˜80 C, thaw in 37° C. water bath): Formulation 1 (Phosphate Buffer Only): 10 mM Phosphate, 100 mM NaCl, 3% (w/v) sucrose, 0.05% (w/v) P188, pH 7.3. Data was collected using a strain gauge and thermocouple.

FIGS. 16A-FIG. 16C depict LVV material compatibility comparing container closure (Schott Type 1 glass vials and West CZ vials) performance over various stability conditions, 1FT=1 cycle of freezing and thawing; 2 hr=2 hr exposure to room temperature; 3× foam is aggressive aspiration and dispensing from a pipette generating visible foam in the container; 10× is the same stability parameters at a 10 fold dilution. LVV was in Formulation 1 (Phosphate Buffer): 10 mM Phosphate, 100 mM NaCl, 3% (w/v) sucrose, 0.05% (w/v) P188, pH 7.3; as measured by functional titer (FIG. 16A), p24 concentration (FIG. 16B), and particle concentration (FIG. 16C). Data was collected using a strain gauge and thermocouple. Particle concentration was obtained using NanoSight.

FIG. 17 depicts a stability study comparing two formulations over 10 cycles of Freezing and Thawing (F/T) and comparing 1 and 3 days at room temperature (RT): Formulation 1 (Phosphate Buffer): 10 mM Phosphate, 100 mM NaCl, 3% (w/v) sucrose, 0.05% (w/v) P188, pH 7.3; Formulation 3 (Histidine Buffer). 20 mM Histidine, 100 mM NaCl, 3% (w/v) sucrose, 0.05% (w/v) P188, pH 6.5.

FIG. 18 depicts a long-term stability (9 month timepoint (9 mo.) stored frozen at −80° C.) data for the Phosphate and Histidine formulations: Formulation 1 (Phosphate): 10 mM Phosphate, 100 mM NaCl, 3% (w/v) sucrose, 0.05% (w/v) P188, pH 7.3; Formulation 3 (Histidine). 20 mM Histidine, 100 mM NaCl, 3% (w/v) sucrose, 0.05% (w/v) P188, pH 6.5.

FIGS. 19A-19D depict stability studies comparing the Phosphate and Histidine buffers at the same pH, 7.0: Formulation 4 (Phosphate): 10 mM Phosphate, 100 mM NaCl, 3% (w/v) sucrose, 0.05% (w/v) P188, pH 7.0; Formulation 5 (Histidine). 20 mM Histidine, 100 mM NaCl, 3% (w/v) sucrose, 0.05% (w/v) P188, pH 7.0; as measured by functional titer (FIG. 19A), normalized functional titer (FIG. 19B), p24 concentration (FIG. 19C), and particle concentration (FIG. 19D). Results are shown in units of functional titer and normalized as a percentage of the starting material (TO). Particle concentration was obtained using the NanoSight.

### DETAILED DESCRIPTION

This disclosure provides, among other things, preparations (formulations) and pharmaceutical compositions of lentiviral vectors (LVs), including recombinant LVs. The disclosure also provides methods of treating a subject with a disorder, including a bleeding disorder, such as hemophilia A or hemophilia B, using LV preparations. The disclosure further provides processes for producing LV preparations.

Generally, nomenclature used in connection with cell and tissue culture, molecular biology, biophysics, immunology, microbiology, genetics, and protein and nucleic acid chemistry described herein is well-known and commonly used in the art. The methods and techniques provided herein are generally performed according to conventional methods well known in the art and as described in various general and more specific references that are cited and discussed throughout the present specification unless otherwise indicated. Enzymatic reactions and purification techniques are performed according to manufacturer's specifications, as commonly accomplished in the art or as described herein. The nomenclature used in connection with, and the laboratory procedures and techniques of, analytical chemistry, synthetic organic chemistry, and medicinal and pharmaceutical chemistry described herein is well-known and commonly used in the art. Standard techniques are used for chemical syntheses, chemical analyses, pharmaceutical preparation, formulation, and delivery, and treatment of patients.

Unless otherwise defined herein, scientific and technical terms used herein have the meanings that are commonly understood by those of ordinary skill in the art. In the event of any latent ambiguity, definitions provided herein take precedence over any dictionary or extrinsic definition. Unless otherwise required by context, singular terms shall include pluralities and plural terms shall include the singular. The use of “or” means “and/or” unless stated otherwise. The use of the term “including,” as well as other forms, such as “includes” and “included,” is not limiting.

So that the invention may be more readily understood, certain terms are first defined.

As used herein, the term “vector” refers to any vehicle for the cloning of and/or transfer of a nucleic acid into a host cell. A vector can be a replicon to which another nucleic acid segment can be attached so as to bring about the replication of the attached segment. A “replicon” refers to any genetic element (e.g., plasmid, phage, cosmid, chromosome, virus) that functions as an autonomous unit of replication in vivo, i.e., capable of replication under its own control. The term “vector” includes both viral and nonviral vehicles for introducing the nucleic acid into a cell in vitro, ex vivo or in vivo. A large number of vectors are known and used in the art including, for example, plasmids, modified eukaryotic viruses, or modified bacterial viruses. Insertion of a polynucleotide into a suitable vector can be accomplished by ligating the appropriate polynucleotide fragments into a chosen vector that has complementary cohesive termini.

As used herein, the phrase “recombinant lentiviral vector” refers to a vector with sufficient lentiviral genetic information to allow packaging of an RNA genome, in the presence of packaging components, into a viral particle capable of infecting a target cell. Infection of the target cell may include reverse transcription and integration into the target cell genome. The recombinant lentiviral vector carries non-viral coding sequences which are to be delivered by the vector to the target cell. A recombinant lentiviral vector is incapable of independent replication to produce infectious lentiviral particles within the final target cell. Usually the recombinant lentiviral vector lacks a functional gag-pol and/or env gene and/or other genes essential for replication. The vector of the present invention may be configured as a split-intron vector.

As used herein, the term “treat” refers to an amelioration or reduction of one or more symptoms of a disorder. Treating need not be a cure.

As used herein, the term “human patient” refers to a human being having a disease or disorder and in need of treatment for this disease or disorder.

As used herein, the phrase “systemically administer” refers to prescribing or giving a pharmaceutical composition comprising an LV to a subject, such that the LV is introduced directly into the bloodstream of the subject. Examples of routes of systemic administration include, but are not limited to, intravenous, e.g., intravenous injection and intravenous infusion, e.g., via central venous access.

As used herein, the term “about,” when used in reference to a particular recited numerical value, means that the value may vary from the recited value by no more than 10%. For example, as used herein, the expression “about 100” includes 90 and 110 and all values in between (e.g., 90, 91, 92, 93, 94, 95, etc.).

### A. Formulations of Lentiviral Vectors (LVs) Comprising TRIS-Free Buffering Systems

In one aspect, the present invention is directed to a recombinant lentiviral vector preparation comprising: (a) an effective dose of a recombinant lentiviral vector; (b) a TRIS-free buffer system; (c) a salt; (d) a surfactant; and (e) a carbohydrate, wherein the pharmaceutical composition is suitable for systemic administration to a human patient. In certain embodiments, the vector comprises a nucleotide sequence encoding VSV-G or a fragment thereof. In certain embodiments, the pH of the buffer system is from about 6.0 to about 8.0. In certain embodiments, the pH of the buffer system is from about 6.0 to about 7.5. In certain embodiments, the pH of the buffer system is from about 6.0 to about 7.0. In certain embodiments, the pH of the buffer system is from about 6.0 to about 8.0. In certain embodiments, the pH of the buffer system is about 6.5.

In certain embodiments, the pH of the buffer system is about 7.3. In certain embodiments, the buffer system is a phosphate buffer or a histidine buffer. In certain embodiments, the concentration of the phosphate or histidine buffer is from about 5 mM to about 30 mM. In certain embodiments, the concentration of the phosphate buffer is from about 10 mM to about 20 mM, from about 10 mM to about 15 mM, from about 20 mM to about 30 mM, from about 20 mM to about 25 mM, or from about 15 mM to about 20 mM. In certain embodiments, the salt is a chloride salt. In certain embodiments, the concentration of the chloride salt is from about 80 mM to about 150 mM. In certain embodiments, the concentration of the salt is about 100 mM, about 110 mM, about 130 mM, or about 150 mM. In certain embodiments, the surfactant is a poloxamer or a polysorbate. In certain embodiments, the concentration of the poloxamer or polysorbate is from about 0.01% (w/v) to about 0.1% (w/v). In certain embodiments, the carbohydrate is sucrose. In certain embodiments, the concentration of the carbohydrate is from about 0.5% (w/v) to about 5% (w/v). In certain embodiments, the chloride salt is sodium chloride (NaCl). In certain embodiments, the poloxamer is selected from the group consisting of poloxamer 101 (P101), poloxamer 105 (P105), poloxamer 108 (P108), poloxamer 122 (P122), poloxamer 123 (P123), poloxamer 124 (P124), poloxamer 181 (P181), poloxamer 182 (P182), poloxamer 183 (P183), poloxamer 184 (P184), poloxamer 185 (P185), poloxamer 188 (P188), poloxamer 212 (P212), poloxamer 215 (P215), poloxamer 217 (P217), poloxamer 231 (P231), poloxamer 234 (P234), poloxamer 235 (P235), poloxamer 237 (P237), poloxamer 238 (P238), poloxamer 282 (P282), poloxamer 284 (P284), poloxamer 288 (P288), poloxamer 331 (P331), poloxamer 333 (P333), poloxamer 334 (P334), poloxamer 335 (P335), poloxamer 338 (P338), poloxamer 401 (P401), poloxamer 402 (P402), poloxamer 403 (P403), poloxamer 407 (P407), and a combination thereof. In certain embodiments, the polysorbate is selected from the group consisting of polysorbate 20, polysorbate 40, polysorbate 60, polysorbate 80, and a combination thereof. In certain embodiments, the pH of the phosphate or histidine buffer is about 6.1, about 6.3, about 6.5, about 6.7, about 6.9, about 7.1, about 7.3, about 7.5, about 7.7, or about 7.9. In certain embodiments, the concentration of the phosphate or histidine buffer is about 10 mM, about 15 mM, about 20 mM, or about 25 mM. In certain embodiments, the chloride salt is about 100 mM, about 110 mM, about 130 mM, or about 150 mM. In certain embodiments, the concentration of the poloxamer or polysorbate is about 0.03% (w/v), about 0.05% (w/v), about 0.07% (w/v), or about 0.09% (w/v). In certain embodiments, the concentration of the carbohydrate is about 1% (w/v), about 2% (w/v), about 3% (w/v), or about 4% (w/v). In certain embodiments, the poloxamer is poloxamer 188 (P188). In certain embodiments, the poloxamer is poloxamer 407 (P407).

In certain embodiments, the present disclosure provides a recombinant lentiviral vector preparation comprising: (a) a therapeutically effective dose of a recombinant lentiviral vector; (b) about 10 mM phosphate; (c) about 100 mM sodium chloride; (d) about 0.05% (w/v) poloxamer 188; and (e) about 3% (w/v) sucrose, wherein the pH of the preparation is about 7.3, and wherein the pharmaceutical composition is suitable for systemic administration to a human patient.

In certain embodiments, the present disclosure provides a recombinant lentiviral vector preparation comprising: (a) a therapeutically effective dose of a recombinant lentiviral vector; (b) about 10 mM phosphate; (c) about 130 mM sodium chloride; (d) about 0.05% (w/v) poloxamer 188; and (e) about 1% (w/v) sucrose, wherein the pH of the preparation is about 7.3, and wherein the pharmaceutical composition is suitable for systemic administration to a human patient.

In certain embodiments, the present disclosure provides a recombinant lentiviral vector preparation comprising: (a) a therapeutically effective dose of a recombinant lentiviral vector; (b) about 20 mM histidine; (c) about 100 mM sodium chloride; (d) about 0.05% (w/v) poloxamer 188; and (e) about 3% (w/v) sucrose, wherein the pH of the preparation is about 6.5, and wherein the pharmaceutical composition is suitable for systemic administration to a human patient.

In certain embodiments, the present disclosure provides a recombinant lentiviral vector preparation comprising: (a) a therapeutically effective dose of a recombinant lentiviral vector; (b) about 10 mM phosphate; (c) about 100 mM sodium chloride; (d) about 0.05% (w/v) poloxamer 188; and (e) about 3% (w/v) sucrose, wherein the pH of the preparation is about 7.0, and wherein the pharmaceutical composition is suitable for systemic administration to a human patient.

In certain embodiments, the present disclosure provides a recombinant lentiviral vector preparation comprising: (a) a therapeutically effective dose of a recombinant lentiviral vector; (b) about 20 mM histidine; (c) about 100 mM sodium chloride; (d) about 0.05% (w/v) poloxamer 188; and (e) about 3% (w/v) sucrose, wherein the pH of the preparation is about 7.0, and wherein the pharmaceutical composition is suitable for systemic administration to a human patient.

### A.1. Lentiviral Vectors

Lentiviral vectors are part of a larger group of retroviral vectors (Coffin et al. (1997) “Retroviruses” Cold Spring Harbor Laboratory Press Eds: J M Coffin, S M Hughes, H E Varmus pp 758-763). Examples of primate lentiviruses include: the human immunodeficiency virus (HIV) and the simian immunodeficiency virus (SIV). The lentivirus family differs from retroviruses in that lentiviruses have the capability to infect both dividing and non-dividing cells (Lewis et al. (1992); Lewis and Emerman (1994)).

A lentiviral vector, as used herein, is a vector which comprises at least one component part derivable from a lentivirus. Preferably, that component part is involved in the biological mechanisms by which the vector infects cells, expresses genes or is replicated. In a recombinant lentiviral vector at least part of one or more protein coding regions essential for replication may be removed from the virus. This makes the viral vector replication-defective. Portions of the viral genome may also be replaced by a transgene, thus rendering the vector capable of transducing a target non-dividing host cell and/or integrating its genome into a host genome.

A recombinant lentiviral is usually pseudotyped. Pseudotyping can confer one or more advantages. For example, the env gene product of the HIV based vectors would restrict these vectors to infecting only cells that express a protein called CD4. But if the env gene in these vectors has been substituted with env sequences from other RNA viruses, then they may have a broader infectious spectrum (Verma and Somia (1997)). The envelope glycoprotein (G) of Vesicular stomatitis virus (VSV), a rhabdovirus, is an envelope protein that has been shown to be capable of pseudotyping certain retroviruses. Pseudotyped VSV-G vectors may be used to transduce a wide range of mammalian cells. The incorporation of a non-lentiviral pseudotyping envelope, such as VSV-G protein gives the advantage that vector particles can be concentrated to a high titre without loss of infectivity (Akkina et al. (1996) J. Virol. 70:2581-5). Lentivirus and retrovirus envelope proteins are apparently unable to withstand the shearing forces during ultracentrifugation, probably because they consist of two non-covalently linked subunits. The interaction between the subunits may be disrupted by the centrifugation. In comparison the VSV glycoprotein is composed of a single unit. VSV-G protein pseudotyping can therefore offer potential advantages.

Lentiviruses include members of the bovine lentivirus group, equine lentivirus group, feline lentivirus group, ovinecaprine lentivirus group, and primate lentivirus group. The development of lentivirus vectors for gene therapy has been reviewed in Klimatcheva et al. (1999) Frontiers in Bioscience 4:481-496. The design and use of lentiviral vectors suitable for gene therapy is described for example in U.S. Pat. Nos. 6,207,455 and 6,615,782. Examples of lentivirus include, but are not limited to, HIV-1, HIV-2, HIV-1/HIV-2 pseudotype, HIV-1/SIV, FIV, caprine arthritis encephalitis virus (CAEV), equine infectious anemia virus, and bovine immunodeficiency virus.

In some embodiments, the lentiviral vector of the present disclosure is a “third-generation” lentiviral vector. As used herein, the term “third-generation” lentiviral vector refers to a lentiviral packaging system that has the characteristics of a second-generation vector system, and that further lacks a functional tat gene, such as one from which the tat gene has been deleted or inactivated. Typically, the gene encoding rev is provided on a separate expression construct. See, e.g., Dull et al. (1998) J. Virol. 72: 8463-8471. As used herein, a “second-generation” lentiviral vector system refers to a lentiviral packaging system that lacks functional accessory genes, such as one from which the accessory genes vif, vpr, vpu, and nef have been deleted or inactivated. See, e.g., Zufferey et al. (1997) Nat. Biotechnol. 15:871-875. As used herein, “packaging system” refers to a set of viral constructs comprising genes that encode viral proteins involved in packaging a recombinant virus. Typically, the constructs of the packaging system will ultimately be incorporated into a packaging cell.

In some embodiments, the third-generation lentiviral vector of the present disclosure is a self-inactivating lentiviral vector. In some embodiments, the lentiviral vector is a VSV.G pseudo type lentiviral vector. In some embodiments, the lentiviral vector comprises a hepatocyte-specific promoter for transgene expression. In some embodiments, the hepatocyte-specific promoter is an enhanced transthyretin promoter. In some embodiments, the lentiviral vector comprises one or more target sequences for miR-142 to reduce immune response to the transgene product. In some embodiments, incorporating one or more target sequences for miR-142 into a lentiviral vector of the present disclosure allows for a desired transgene expression profile. For example, incorporating one or more target sequences for miR-142 may suppress transgene expression in intravascular and extravascular hematopoietic lineages, whereas transgene expression is maintained in nonhematopoietic cells. No oncogenesis has been detected in tumor prone mice treated with the lentivirus vector system of the present disclosure. See Brown et al. (2007) Blood 110:4144-52, Brown at al. (2006) Nat. Ned. 12:585-91, and Cantore et al. (2015) Sci. Transl. Med. 7(277):277ra28.

Lentiviral vectors of the disclosure include codon optimized polynucleotides of transgenes encoding specific proteins, such as the FVIII or FIX protein described herein. In one embodiment, the optimized coding sequences for the FVIII or FIX protein is operably linked to an expression control sequence. As used herein, two nucleic acid sequences are operably linked when they are covalently linked in such a way as to permit each component nucleic acid sequence to retain its functionality. A coding sequence and a gene expression control sequence are said to be operably linked when they are covalently linked in such a way as to place the expression or transcription and/or translation of the coding sequence under the influence or control of the gene expression control sequence. Two DNA sequences are said to be operably linked if induction of a promoter in the 5′ gene expression sequence results in the transcription of the coding sequence and if the nature of the linkage between the two DNA sequences does not (1) result in the introduction of a frame-shift mutation, (2) interfere with the ability of the promoter region to direct the transcription of the coding sequence, or (3) interfere with the ability of the corresponding RNA transcript to be translated into a protein. Thus, a gene expression sequence would be operably linked to a coding nucleic acid sequence if the gene expression sequence were capable of effecting transcription of that coding nucleic acid sequence such that the resulting transcript is translated into the desired protein or polypeptide.

In certain embodiments, the lentiviral vector is a vector of a recombinant lentivirus capable of infecting non-dividing cells. In certain embodiments, the lentiviral vector is a vector of a recombinant lentivirus capable of infecting liver cells (e.g., hepatocytes). The lentiviral genome and the proviral DNA typically have the three genes found in retroviruses: gag, pol and env, which are flanked by two long terminal repeat (LTR) sequences. The gag gene encodes the internal structural (matrix, capsid and nucleocapsid) proteins; the pol gene encodes the RNA-directed DNA polymerase (reverse transcriptase), a protease and an integrase; and the env gene encodes viral envelope glycoproteins. The 5′ and 3′ LTR's serve to promote transcription and polyadenylation of the virion RNA's. The LTR contains all other cis-acting sequences necessary for viral replication. Lentiviruses have additional genes including vif, vpr, tat, rev, vpu, nef and vpx (in HIV-1, HIV-2 and/or SIV).

Adjacent to the 5′ LTR are sequences necessary for reverse transcription of the genome (the tRNA primer binding site) and for efficient encapsidation of viral RNA into particles (the Psi site). If the sequences necessary for encapsidation (or packaging of retroviral RNA into infectious virions) are missing from the viral genome, the cis defect prevents encapsidation of genomic RNA.

However, the resulting mutant remains capable of directing the synthesis of all virion proteins. The disclosure provides a method of producing a recombinant lentivirus capable of infecting a non-dividing cell comprising transfecting a suitable host cell with two or more vectors carrying the packaging functions, namely gag, pol and env, as well as rev and tat. As will be disclosed herein below, vectors lacking a functional tat gene are desirable for certain applications. Thus, for example, a first vector can provide a nucleic acid encoding a viral gag and a viral pol and another vector can provide a nucleic acid encoding a viral env to produce a packaging cell. Introducing a vector providing a heterologous gene, herein identified as a transfer vector, into that packaging cell yields a producer cell which releases infectious viral particles carrying the foreign gene of interest.

According to the above-indicated configuration of vectors and foreign genes, the second vector can provide a nucleic acid encoding a viral envelope (env) gene. The env gene can be derived from nearly any suitable virus, including retroviruses. In some embodiments, the env protein is an amphotropic envelope protein which allows transduction of cells of human and other species.

Examples of retroviral-derived env genes include, but are not limited to: Moloney murine leukemia virus (MoMuLV or MMLV), Harvey murine sarcoma virus (HaMuSV or HSV), murine mammary tumor virus (MuMTV or MMTV), gibbon ape leukemia virus (GaLV or GALV), human immunodeficiency virus (HIV) and Rous sarcoma virus (RSV). Other env genes such as Vesicular stomatitis virus (VSV) protein G (VSV-G), that of hepatitis viruses and of influenza also can be used. In some embodiments, the viral env nucleic acid sequence is associated operably with regulatory sequences described elsewhere herein. In certain embodiments, a formulation buffer of the present disclosure confers lentivirus stability and affords long term frozen storage, in particular, for lentivirus comprising VSV-G. A formulation buffer of the present invention offers enhanced lentivirus stability upon freezing and thawing as well as exposure to elevated temperatures, in particular, for lentivirus comprising VSV-G.

In certain embodiments, the lentiviral vector has the HIV virulence genes env, vif, vpr, vpu and nef deleted without compromising the ability of the vector to transduce non-dividing cells. In some embodiments, the lentiviral vector comprises a deletion of the U3 region of the 3′ LTR. The deletion of the U3 region can be the complete deletion or a partial deletion.

In some embodiments, the lentiviral vector of the disclosure comprising the FVIII nucleotide sequence described herein can be transfected in a cell with (a) a first nucleotide sequence comprising a gag, a pol, or gag and pol genes and (b) a second nucleotide sequence comprising a heterologous env gene; wherein the lentiviral vector lacks a functional tat gene. In other embodiments, the cell is further transfected with a fourth nucleotide sequence comprising a rev gene. In certain embodiments, the lentiviral vector lacks functional genes selected from vif, vpr, vpu, vpx and nef, or a combination thereof.

In certain embodiments, a lentiviral vector of the instant disclosure comprises one or more nucleotide sequences encoding a gag protein, a Rev-response element, a central polypurine track (cPPT), or any combination thereof.

In some embodiments, the lentiviral vector expresses on its surface one or more polypeptides that improve the targeting and/or activity of the lentiviral vector or the encoded FVIII polypeptide. The one or more polypeptides can be encoded by the lentiviral vector or can be incorporated during budding of the lentiviral vector from a host cell. During lentiviral production, viral particles bud off from a producing host cell. During the budding process, the viral particle takes on a lipid coat, which is derived from the lipid membrane of the host cell. As a result, the lipid coat of the viral particle can include membrane bound polypeptides that were previously present on the surface of the host cell.

In some embodiments, the lentiviral vector expresses one or more polypeptides on its surface that inhibit an immune response to the lentiviral vector following administration to a human subject. In some embodiments, the surface of the lentiviral vector comprises one or more CD47 molecules. CD47 is a “marker of self” protein, which is ubiquitously expressed on human cells. Surface expression of CD47 inhibits macrophage-induced phagocytosis of endogenous cells through the interaction of CD47 and macrophage expressed-SIRPα. Cells expressing high levels of CD47 are less likely to be targeted and destroyed by human macrophages in vivo.

In some embodiments, the lentiviral vector comprises a high concentration of CD47 polypeptide molecules on its surface. In some embodiments, the lentiviral vector is produced in a cell line that has a high expression level of CD47. In certain embodiments, the lentiviral vector is produced in a CD47^{high} cell, wherein the cell has high expression of CD47 on the cell membrane. In particular embodiments, the lentiviral vector is produced in a CD47^{high} HEK 293T cell, wherein the HEK 293T is has high expression of CD47 on the cell membrane. In some embodiments, the HEK 293T cell is modified to have increased expression of CD47 relative to unmodified HEK 293T cells. In certain embodiments, the CD47 is human CD47.

In some embodiments, the lentiviral vector has little or no surface expression of major histocompatibility complex class I (MHC-I). Surface expressed MHC-I displays peptide fragments of “non-self” proteins from within a cell, such as protein fragments indicative of an infection, facilitating an immune response against the cell. In some embodiments, the lentiviral vector is produced in a MHC-I^{low} cell, wherein the cell has reduced expression of MHC-I on the cell membrane. In some embodiments, the lentiviral vector is produced in an MHC-I⁻ (or “MHC-I^{free}”, “MHC-1^{neg}” or “MHC-negative”) cell, wherein the cell lacks expression of MHC-I.

In particular embodiments, the lentiviral vector comprises a lipid coat comprising a high concentration of CD47 polypeptides and lacking MHC-T polypeptides. In certain embodiments, the lentiviral vector is produced in a C47^{high}/MHC-I^{low} cell line, e.g., a CD47^{high}/MHC-I^{low} HEK 293T cell line. In some embodiments, the lentiviral vector is produced in a CD47^{high}/MHC-I^{low} cell line, e.g., a CD47^{high}/MHC-I^{free} HEK 293T cell line.

Examples of lentiviral vectors are disclosed in U.S. Pat. No. 9,050,269 and International Publication Nos. WO9931251, WO9712622, WO9817815, WO9817816, and WO9818934, which are incorporated herein by reference in their entireties.

In some embodiments, the present disclosure provides a lentiviral vector comprising an isolated nucleic acid molecule comprising a nucleotide sequence which comprises a nucleotide sequence as shown in Table 1.

TABLE 1
Sequences

| SEQ | | |
|---|---|---|
| ID | | |
| NO | Description | Sequence |
| 1 | FVIII coding | ATGCAGATTGAGCTGTCCACTTGTTTCTTCCTGTGCCTCCTGC |
| | sequence | GCTTCTGTTTCTCCGCCACTCGCCGGTACTACCTTGGAGCCGT |
| | | GGAGCTTTCATGGGACTACATGCAGAGCGACCTGGGCGAAC |
| | | TCCCCGTGGATGCCAGATTCCCCCCCCGCGTGCCAAAGTCCT |
| | | TCCCCTTTAACACCTCCGTGGTGTACAAGAAAACCCTCTTTG |
| | | TCGAGTTCACTGACCACCTGTTCAACATCGCCAAGCCGCGCC |
| | | CACCTTGGATGGGCCTCCTGGGACCGACCATTCAAGCTGAAG |
| | | TGTACGACACCGTGGTGATCACCCTGAAGAACATGGCGTCCC |
| | | ACCCCGTGTCCCTGCATGCGGTCGGAGTGTCCTACTGGAAGG |
| | | CCTCCGAAGGAGCTGAGTACGACGACCAGACTAGCCAGCGG |
| | | GAAAAGGAGGACGATAAAGTGTTCCCGGGCGGCTCGCATAC |
| | | TTACGTGTGGCAAGTCCTGAAGGAAAACGGACCTATGGCAT |
| | | CCGATCCTCTGTGCCTGACTTACTCCTACCTTTCCCATGTGGA |
| | | CCTCGTGAAGGACCTGAACAGCGGGCTGATTGGTGCACTTCT |
| | | CGTGTGCCGCGAAGGTTCGCTCGCTAAGGAAAAGACCCAGA |
| | | CCCTCCATAAGTTCATCCTTTTGTTCGCTGTGTTCGATGAAGG |
| | | AAAGTCATGGCATTCCGAAACTAAGAACTCGCTGATGCAGG |
| | | ACCGGGATGCCGCCTCAGCCCGCGCCTGGCCTAAAATGCAT |
| | | ACAGTCAACGGATACGTGAATCGGTCACTGCCCGGGCTCATC |
| | | GGTTGTCACAGAAAGTCCGTGTACTGGCACGTCATCGGCATG |
| | | GGCACTACGCCTGAAGTGCACTCCATCTTCCTGGAAGGGCAC |
| | | ACCTTCCTCGTGCGCAACCACCGCCAGGCCTCTCTGGAAATC |
| | | TCCCCGATTACCTTTCTGACCGCCCAGACTCTGCTCATGGAC |
| | | CTGGGGCAGTTCCTTCTCTTCTGCCACATCTCCAGCCATCAG |
| | | CACGACGGAATGGAGGCCTACGTGAAGGTGGACTCATGCCC |
| | | GGAAGAACCTCAGTTGCGGATGAAGAACAACGAGGAGGCCG |
| | | AGGACTATGACGACGATTTGACTGACTCCGAGATGGACGTC |
| | | GTGCGGTTCGATGACGACAACAGCCCCAGCTTCATCCAGATT |
| | | CGCAGCGTGGCCAAGAAGCACCCCAAAACCTGGGTGCACTA |
| | | CATCGCGGCCGAGGAAGAAGATTGGGACTACGCCCCGTTGG |
| | | TGCTGGCACCCGATGACCGGTCGTACAAGTCCCAGTATCTGA |
| | | ACAATGGTCCGCAGCGGATTGGCAGAAAGTACAAGAAAGTG |
| | | CGGTTCATGGCGTACACTGACGAAACGTTTAAGACCCGGGA |
| | | GGCCATTCAACATGAGAGCGGCATTCTGGGACCACTGCTGTA |
| | | CGGAGAGGTCGGCGATACCCTGCTCATCATCTTCAAAAACCA |
| | | GGCCTCCCGGCCTTACAACATCTACCCTCACGGAATCACCGA |
| | | CGTGCGGCCACTCTACTCGCGGCGCCTGCCGAAGGGCGTCA |
| | | AGCACCTGAAAGACTTCCCTATCCTGCCGGGCGAAATCTTCA |
| | | AGTATAAGTGGACCGTCACCGTGGAGGACGGGCCCACCAAG |
| | | AGCGATCCTAGGTGTCTGACTCGGTACTACTCCAGCTTCGTG |
| | | AACATGGAACGGGACCTGGCATCGGGACTCATTGGACCGCT |
| | | GCTGATCTGCTACAAAGAGTCGGTGGATCAACGCGGCAACC |
| | | AGATCATGTCCGACAAGCGCAACGTGATCCTGTTCTCCGTGT |
| | | TTGATGAAAACAGATCCTGGTACCTCACTGAAAACATCCAG |
| | | AGGTTCCTCCCAAACCCCGCAGGAGTGCAACTGGAGGACCC |
| | | TGAGTTTCAGGCCTCGAATATCATGCACTCGATTAACGGTTA |
| | | CGTGTTCGACTCGCTGCAGCTGAGCGTGTGCCTCCATGAAGT |
| | | CGCTTACTGGTACATTCTGTCCATCGGCGCCCAGACTGACTT |
| | | CCTGAGCGTGTTCTTTTCCGGTTACACCTTTAAGCACAAGAT |
| | | GGTGTACGAAGATACCCTGACCCTGTTCCCTTTCTCCGGCGA |
| | | AACGGTGTTCATGTCGATGGAGAACCCGGGTCTGTGGATTCT |
| | | GGGATGCCACAACAGCGACTTTCGGAACCGCGGAATGACTG |
| | | CCCTGCTGAAGGTGTCCTCATGCGACAAGAACACCGGAGAC |
| | | TACTACGAGGACTCCTACGAGGATATCTCAGCCTACCTCCTG |
| | | TCCAAGAACAACGCGATCGAGCCGCGCAGCTTCAGCCAGAA |
| | | CCCGCCTGTGCTGAAGAGGCACCAGCGAGAAATTACCCGGA |
| | | CCACCCTCCAATCGGATCAGGAGGAAATCGACTACGACGAC |
| | | ACCATCTCGGTGGAAATGAAGAAGGAAGATTTCGATATCTA |
| | | CGACGAGGACGAAAATCAGTCCCCTCGCTCATTCCAAAAGA |
| | | AAACTAGACACTACTTTATCGCCGCGGTGGAAAGACTGTGG |
| | | GACTATGGAATGTCATCCAGCCCTCACGTCCTTCGGAACCGG |
| | | GCCCAGAGCGGATCGGTGCCTCAGTTCAAGAAAGTGGTGTT |
| | | CCAGGAGTTCACCGACGGCAGCTTCACCCAGCCGCTGTACC |
| | | GGGGAGAACTGAACGAACACCTGGGCCTGCTCGGTCCCTAC |
| | | ATCCGCGCGGAAGTGGAGGATAACATCATGGTGACCTTCCG |
| | | TAACCAAGCATCCAGACCTTACTCCTTCTATTCCTCCCTGATC |
| | | TCATACGAGGAGGACCAGCGCCAAGGCGCCGAGCCCCGCAA |
| | | GAACTTCGTCAAGCCCAACGAGACTAAGACCTACTTCTGGA |
| | | AGGTCCAACACCATATGGCCCCGACCAAGGATGAGTTTGAC |
| | | TGCAAGGCCTGGGCCTACTTCTCCGACGTGGACCTTGAGAAG |
| | | GATGTCCATTCCGGCCTGATCGGGCCGCTGCTCGTGTGTCAC |
| | | ACCAACACCCTGAACCCAGCGCATGGACGCCAGGTCACCGT |
| | | CCAGGAGTTTGCTCTGTTCTTCACCATTTTTGACGAAACTAA |
| | | GTCCTGGTACTTCACCGAGAATATGGAGCGAAACTGTAGAG |
| | | CGCCCTGCAATATCCAGATGGAAGATCCGACTTTCAAGGAG |
| | | AACTATAGATTCCACGCCATCAACGGGTACATCATGGATACT |
| | | CTGCCGGGGCTGGTCATGGCCCAGGATCAGAGGATTCGGTG |
| | | GTACTTGCTGTCAATGGGATCGAACGAAAACATTCACTCCAT |
| | | TCACTTCTCCGGTCACGTGTTCACTGTGCGCAAGAAGGAGGA |
| | | GTACAAGATGGCGCTGTACAATCTGTACCCCGGGGTGTTCGA |
| | | AACTGTGGAGATGCTGCCGTCCAAGGCCGGCATCTGGAGAG |
| | | TGGAGTGCCTGATCGGAGAGCACCTCCACGCGGGGATGTCC |
| | | ACCCTCTTCCTGGTGTACTCGAATAAGTGCCAGACCCCGCTG |
| | | GGCATGGCCTCGGGCCACATCAGAGACTTCCAGATCACAGC |
| | | AAGCGGACAATACGGCCAATGGGCGCCGAAGCTGGCCCGCT |
| | | TGCACTACTCCGGATCGATCAACGCATGGTCCACCAAGGAA |
| | | CCGTTCTCGTGGATTAAGGTGGACCTCCTGGCCCCTATGATT |
| | | ATCCACGGAATTAAGACCCAGGGCGCCAGGCAGAAGTTCTC |
| | | CTCCCTGTACATCTCGCAATTCATCATCATGTACAGCCTGGA |
| | | CGGGAAGAAGTGGCAGACTTACAGGGGAAACTCCACCGGCA |
| | | CCCTGATGGTCTTTTTCGGCAACGTGGATTCCTCCGGCATTA |
| | | AGCACAACATCTTCAACCCACCGATCATAGCCAGATATATTA |
| | | GGCTCCACCCCACTCACTACTCAATCCGCTCAACTCTTCGGA |
| | | TGGAACTCATGGGGTGCGACCTGAACTCCTGCTCCATGCCGT |
| | | TGGGGATGGAATCAAAGGCTATTAGCGACGCCCAGATCACC |
| | | GCGAGCTCCTACTTCACTAACATGTTCGCCACCTGGAGCCCC |
| | | TCCAAGGCCAGGCTGCACTTGCAGGGACGGTCAAATGCCTG |
| | | GCGGCCGCAAGTGAACAATCCGAAGGAATGGCTTCAAGTGG |
| | | ATTTCCAAAAGACCATGAAAGTGACCGGAGTCACCACCCAG |
| | | GGAGTGAAGTCCCTTCTGACCTCGATGTATGTGAAGGAGTTC |
| | | CTGATTAGCAGCAGCCAGGACGGGCACCAGTGGACCCTGTT |
| | | CTTCCAAAACGGAAAGGTCAAGGTGTTCCAGGGGAACCAGG |
| | | ACTCGTTCACACCCGTGGTGAACTCCCTGGACCCCCCACTGC |
| | | TGACGCGGTACTTGAGGATTCATCCTCAGTCCTGGGTCCATC |
| | | AGATTGCATTGCGAATGGAAGTCCTGGGCTGCGAGGCCCAG |
| | | GACCTGTAC |
| 2 | FVIII coding | ATGCAGATTGAGCTGTCCACTTGTTTCTTCCTGTGCCTCCTGC |
| | sequence | GCTTCTGTTTCTCCGCCACTCGCCGGTACTACCTTGGAGCCGT |
| | comprising | GGAGCTTTCATGGGACTACATGCAGAGCGACCTGGGCGAAC |
| | XTEN | TCCCCGTGGATGCCAGATTCCCCCCCCGCGTGCCAAAGTCCT |
| | (XTEN in | TCCCCTTTAACACCTCCGTGGTGTACAAGAAAACCCTCTTTG |
| | bold and | TCGAGTTCACTGACCACCTGTTCAACATCGCCAAGCCGCGCC |
| | underline) | CACCTTGGATGGGCCTCCTGGGACCGACCATTCAAGCTGAAG |
| | | TGTACGACACCGTGGTGATCACCCTGAAGAACATGGCGTCCC |
| | | ACCCCGTGTCCCTGCATGCGGTCGGAGTGTCCTACTGGAAGG |
| | | CCTCCGAAGGAGCTGAGTACGACGACCAGACTAGCCAGCGG |
| | | GAAAAGGAGGACGATAAAGTGTTCCCGGGCGGCTCGCATAC |
| | | TTACGTGTGGCAAGTCCTGAAGGAAAACGGACCTATGGCAT |
| | | CCGATCCTCTGTGCCTGACTTACTCCTACCTTTCCCATGTGGA |
| | | CCTCGTGAAGGACCTGAACAGCGGGCTGATTGGTGCACTTCT |
| | | CGTGTGCCGCGAAGGTTCGCTCGCTAAGGAAAAGACCCAGA |
| | | CCCTCCATAAGTTCATCCTTTTGTTCGCTGTGTTCGATGAAGG |
| | | AAAGTCATGGCATTCCGAAACTAAGAACTCGCTGATGCAGG |
| | | ACCGGGATGCCGCCTCAGCCCGCGCCTGGCCTAAAATGCAT |
| | | ACAGTCAACGGATACGTGAATCGGTCACTGCCCGGGCTCATC |
| | | GGTTGTCACAGAAAGTCCGTGTACTGGCACGTCATCGGCATG |
| | | GGCACTACGCCTGAAGTGCACTCCATCTTCCTGGAAGGGCAC |
| | | ACCTTCCTCGTGCGCAACCACCGCCAGGCCTCTCTGGAAATC |
| | | TCCCCGATTACCTTTCTGACCGCCCAGACTCTGCTCATGGAC |
| | | CTGGGGCAGTTCCTTCTCTTCTGCCACATCTCCAGCCATCAG |
| | | CACGACGGAATGGAGGCCTACGTGAAGGTGGACTCATGCCC |
| | | GGAAGAACCTCAGTTGCGGATGAAGAACAACGAGGAGGCCG |
| | | AGGACTATGACGACGATTTGACTGACTCCGAGATGGACGTC |
| | | GTGCGGTTCGATGACGACAACAGCCCCAGCTTCATCCAGATT |
| | | CGCAGCGTGGCCAAGAAGCACCCCAAAACCTGGGTGCACTA |
| | | CATCGCGGCCGAGGAAGAAGATTGGGACTACGCCCCGTTGG |
| | | TGCTGGCACCCGATGACCGGTCGTACAAGTCCCAGTATCTGA |
| | | ACAATGGTCCGCAGCGGATTGGCAGAAAGTACAAGAAAGTG |
| | | CGGTTCATGGCGTACACTGACGAAACGTTTAAGACCCGGGA |
| | | GGCCATTCAACATGAGAGCGGCATTCTGGGACCACTGCTGTA |
| | | CGGAGAGGTCGGCGATACCCTGCTCATCATCTTCAAAAACCA |
| | | GGCCTCCCGGCCTTACAACATCTACCCTCACGGAATCACCGA |
| | | CGTGCGGCCACTCTACTCGCGGCGCCTGCCGAAGGGCGTCA |
| | | AGCACCTGAAAGACTTCCCTATCCTGCCGGGCGAAATCTTCA |
| | | AGTATAAGTGGACCGTCACCGTGGAGGACGGGCCCACCAAG |
| | | AGCGATCCTAGGTGTCTGACTCGGTACTACTCCAGCTTCGTG |
| | | AACATGGAACGGGACCTGGCATCGGGACTCATTGGACCGCT |
| | | GCTGATCTGCTACAAAGAGTCGGTGGATCAACGCGGCAACC |
| | | AGATCATGTCCGACAAGCGCAACGTGATCCTGTTCTCCGTGT |
| | | TTGATGAAAACAGATCCTGGTACCTCACTGAAAACATCCAG |
| | | AGGTTCCTCCCAAACCCCGCAGGAGTGCAACTGGAGGACCC |
| | | TGAGTTTCAGGCCTCGAATATCATGCACTCGATTAACGGTTA |
| | | CGTGTTCGACTCGCTGCAGCTGAGCGTGTGCCTCCATGAAGT |
| | | CGCTTACTGGTACATTCTGTCCATCGGCGCCCAGACTGACTT |
| | | CCTGAGCGTGTTCTTTTCCGGTTACACCTTTAAGCACAAGAT |
| | | GGTGTACGAAGATACCCTGACCCTGTTCCCTTTCTCCGGCGA |
| | | AACGGTGTTCATGTCGATGGAGAACCCGGGTCTGTGGATTCT |
| | | GGGATGCCACAACAGCGACTTTCGGAACCGCGGAATGACTG |
| | | CCCTGCTGAAGGTGTCCTCATGCGACAAGAACACCGGAGAC |
| | | TACTACGAGGACTCCTACGAGGATATCTCAGCCTACCTCCTG |
| | | TCCAAGAACAACGCGATCGAGCCGCGCAGCTTCAGCCAGAA |
| | | CACATCAGAGAGCGCCACCCCTGAAAGTGGTCCCGGGAG |
| | | CGAGCCAGCCACATCTGGGTCGGAAACGCCAGGCACAAG |
| | | TGAGTCTGCAACTCCCGAGTCCGGACCTGGCTCCGAGCC |
| | | TGCCACTAGCGGCTCCGAGACTCCGGGAACTTCCGAGAG |
| | | CGCTACACCAGAAAGCGGACCCGGAACCAGTACCGAACC |
| | | TAGCGAGGGCTCTGCTCCGGGCAGCCCAGCCGGCTCTCC |
| | | TACATCCACGGAGGAGGGCACTTCCGAATCCGCCACCCC |
| | | GGAGTCAGGGCCAGGATCTGAACCCGCTACCTCAGGCAG |
| | | TGAGACGCCAGGAACGAGCGAGTCCGCTACACCGGAGA |
| | | GTGGGCCAGGGAGCCCTGCTGGATCTCCTACGTCCACTG |
| | | AGGAAGGGTCACCAGCGGGCTCGCCCACCAGCACTGAAG |
| | | AAGGTGCCTCGAGCCCGCCTGTGCTGAAGAGGCACCAGCG |
| | | AGAAATTACCCGGACCACCCTCCAATCGGATCAGGAGGAAA |
| | | TCGACTACGACGACACCATCTCGGTGGAAATGAAGAAGGAA |
| | | GATTTCGATATCTACGACGAGGACGAAAATCAGTCCCCTCGC |
| | | TCATTCCAAAAGAAAACTAGACACTACTTTATCGCCGCGGTG |
| | | GAAAGACTGTGGGACTATGGAATGTCATCCAGCCCTCACGTC |
| | | CTTCGGAACCGGGCCCAGAGCGGATCGGTGCCTCAGTTCAA |
| | | GAAAGTGGTGTTCCAGGAGTTCACCGACGGCAGCTTCACCC |
| | | AGCCGCTGTACCGGGGAGAACTGAACGAACACCTGGGCCTG |
| | | CTCGGTCCCTACATCCGCGCGGAAGTGGAGGATAACATCAT |
| | | GGTGACCTTCCGTAACCAAGCATCCAGACCTTACTCCTTCTA |
| | | TTCCTCCCTGATCTCATACGAGGAGGACCAGCGCCAAGGCGC |
| | | CGAGCCCCGCAAGAACTTCGTCAAGCCCAACGAGACTAAGA |
| | | CCTACTTCTGGAAGGTCCAACACCATATGGCCCCGACCAAGG |
| | | ATGAGTTTGACTGCAAGGCCTGGGCCTACTTCTCCGACGTGG |
| | | ACCTTGAGAAGGATGTCCATTCCGGCCTGATCGGGCCGCTGC |
| | | TCGTGTGTCACACCAACACCCTGAACCCAGCGCATGGACGC |
| | | CAGGTCACCGTCCAGGAGTTTGCTCTGTTCTTCACCATTTTTG |
| | | ACGAAACTAAGTCCTGGTACTTCACCGAGAATATGGAGCGA |
| | | AACTGTAGAGCGCCCTGCAATATCCAGATGGAAGATCCGAC |
| | | TTTCAAGGAGAACTATAGATTCCACGCCATCAACGGGTACAT |
| | | CATGGATACTCTGCCGGGGCTGGTCATGGCCCAGGATCAGA |
| | | GGATTCGGTGGTACTTGCTGTCAATGGGATCGAACGAAAAC |
| | | ATTCACTCCATTCACTTCTCCGGTCACGTGTTCACTGTGCGCA |
| | | AGAAGGAGGAGTACAAGATGGCGCTGTACAATCTGTACCCC |
| | | GGGGTGTTCGAAACTGTGGAGATGCTGCCGTCCAAGGCCGG |
| | | CATCTGGAGAGTGGAGTGCCTGATCGGAGAGCACCTCCACG |
| | | CGGGGATGTCCACCCTCTTCCTGGTGTACTCGAATAAGTGCC |
| | | AGACCCCGCTGGGCATGGCCTCGGGCCACATCAGAGACTTC |
| | | CAGATCACAGCAAGCGGACAATACGGCCAATGGGCGCCGAA |
| | | GCTGGCCCGCTTGCACTACTCCGGATCGATCAACGCATGGTC |
| | | CACCAAGGAACCGTTCTCGTGGATTAAGGTGGACCTCCTGGC |
| | | CCCTATGATTATCCACGGAATTAAGACCCAGGGCGCCAGGC |
| | | AGAAGTTCTCCTCCCTGTACATCTCGCAATTCATCATCATGT |
| | | ACAGCCTGGACGGGAAGAAGTGGCAGACTTACAGGGGAAAC |
| | | TCCACCGGCACCCTGATGGTCTTTTTCGGCAACGTGGATTCC |
| | | TCCGGCATTAAGCACAACATCTTCAACCCACCGATCATAGCC |
| | | AGATATATTAGGCTCCACCCCACTCACTACTCAATCCGCTCA |
| | | ACTCTTCGGATGGAACTCATGGGGTGCGACCTGAACTCCTGC |
| | | TCCATGCCGTTGGGGATGGAATCAAAGGCTATTAGCGACGC |
| | | CCAGATCACCGCGAGCTCCTACTTCACTAACATGTTCGCCAC |
| | | CTGGAGCCCCTCCAAGGCCAGGCTGCACTTGCAGGGACGGT |
| | | CAAATGCCTGGCGGCCGCAAGTGAACAATCCGAAGGAATGG |
| | | CTTCAAGTGGATTTCCAAAAGACCATGAAAGTGACCGGAGT |
| | | CACCACCCAGGGAGTGAAGTCCCTTCTGACCTCGATGTATGT |
| | | GAAGGAGTTCCTGATTAGCAGCAGCCAGGACGGGCACCAGT |
| | | GGACCCTGTTCTTCCAAAACGGAAAGGTCAAGGTGTTCCAG |
| | | GGGAACCAGGACTCGTTCACACCCGTGGTGAACTCCCTGGA |
| | | CCCCCCACTGCTGACGCGGTACTTGAGGATTCATCCTCAGTC |
| | | CTGGGTCCATCAGATTGCATTGCGAATGGAAGTCCTGGGCTG |
| | | CGAGGCCCAGGACCTGTAC |
| 3 | FIX-R338L | ATGCAGAGAGTCAACATGATTATGGCTGAGTCACCTGGG |
| | coding | CTGATTACTATTTGCCTGCTGGGCTACCTGCTGTCCGCC |
| | sequence | GAGTGTACCGTGTTCCTGGACCATGAGAACGCAAATAAG |
| | (signal | ATCCTGAACAGGCCCAAAAGATACAATAGTGGGAAGCTGG |
| | peptide in | AGGAATTTGTGCAGGGCAACCTGGAGAGAGAATGCATGGAG |
| | bold and | GAAAAGTGTAGCTTCGAGGAAGCCCGCGAGGTGTTTGAAAA |
| | underline) | TACAGAGCGAACCACAGAGTTCTGGAAGCAGTATGTGGACG |
| | | GCGATCAGTGCGAGAGCAACCCCTGTCTGAATGGCGGAAGT |
| | | TGCAAAGACGATATCAACTCATACGAATGCTGGTGTCCTTTC |
| | | GGGTTTGAAGGCAAAAATTGCGAGCTGGACGTGACATGTAA |
| | | CATTAAGAATGGACGGTGCGAGCAGTTTTGTAAAAACTCTGC |
| | | CGATAATAAGGTGGTGTGCAGCTGTACTGAAGGATATCGCCT |
| | | GGCTGAGAACCAGAAGTCCTGCGAACCAGCAGTGCCCTTCC |
| | | CTTGTGGGAGGGTGAGCGTCTCCCAGACTTCAAAACTGACCA |
| | | GAGCAGAGACAGTGTTTCCCGACGTGGATTACGTCAACAGC |
| | | ACTGAGGCCGAAACCATCCTGGACAACATTACTCAGTCTACC |
| | | CAGAGTTTCAATGACTTTACTCGGGTGGTCGGGGGCGAGGAT |
| | | GCTAAACCAGGCCAGTTCCCCTGGCAGGTGGTCCTGAACGG |
| | | AAAGGTGGATGCATTTTGCGGAGGGTCTATCGTGAATGAGA |
| | | AATGGATTGTCACCGCCGCTCACTGCGTGGAAACCGGAGTC |
| | | AAGATCACAGTGGTCGCTGGGGAGCACAACATTGAGGAAAC |
| | | AGAACATACTGAGCAGAAGCGGAATGTGATCCGCATCATTC |
| | | CTCACCATAACTACAATGCAGCCATCAACAAATACAATCATG |
| | | ACATTGCCCTGCTGGAACTGGATGAGCCTCTGGTGCTGAACA |
| | | GCTACGTCACTCCAATCTGCATTGCTGACAAAGAGTATACCA |
| | | ATATCTTCCTGAAGTTTGGATCAGGGTACGTGAGCGGCTGGG |
| | | GAAGAGTCTTCCACAAGGGCAGGAGCGCCCTGGTGCTCCAG |
| | | TATCTGCGAGTGCCTCTGGTCGATCGAGCTACCTGTCTGCTC |
| | | TCTACCAAGTTTACAATCTACAACAACATGTTCTGCGCTGGG |
| | | TTTCACGAGGGAGGACGAGACTCCTGTCAGGGCGATTCTGG |
| | | GGGCCCACATGTGACAGAGGTCGAAGGCACCAGCTTCCTGA |
| | | CTGGCATCATTTCCTGGGGAGAGGAATGTGCAATGAAGGGA |
| | | AAATACGGGATCTACACCAAAGTGAGCCGCTATGTGAACTG |
| | | GATCAAGGAAAAAACCAAACTGACC |
| 4 | Mature FVIII | ATRRYYLGAVELSWDYMQSDLGELPVDARFPPRVPKSFPFNTS |
| | polypeptide | VVYKKTLFVEFTDHLFNIAKPRPPWMGLLGPTIQAEVYDTVVIT |
| | | LKNMASHPVSLHAVGVSYWKASEGAEYDDQTSQREKEDDKVF |
| | | PGGSHTYVWQVLKENGPMASDPLCLTYSYLSHVDLVKDLNSG |
| | | LIGALLVCREGSLAKEKTQTLHKFILLFAVFDEGKSWHSETKNS |
| | | LMQDRDAASARAWPKMHTVNGYVNRSLPGLIGCHRKSVYWH |
| | | VIGMGTTPEVHSIFLEGHTFLVRNHRQASLEISPITFLTAQTLLM |
| | | DLGQFLLFCHISSHQHDGMEAYVKVDSCPEEPQLRMKNNEEAE |
| | | DYDDDLTDSEMDVVRFDDDNSPSFIQIRSVAKKHPKTWVHYIA |
| | | AEEEDWDYAPLVLAPDDRSYKSQYLNNGPQRIGRKYKKVRFM |
| | | AYTDETFKTREAIQHESGILGPLLYGEVGDTLLIIFKNQASRPYNI |
| | | YPHGITDVRPLYSRRLPKGVKHLKDFPILPGEIFKYKWTVTVED |
| | | GPTKSDPRCLTRYYSSFVNMERDLASGLIGPLLICYKESVDQRG |
| | | NQIMSDKRNVILFSVFDENRSWYLTENIQRFLPNPAGVQLEDPE |
| | | FQASNIMHSINGYVFDSLQLSVCLHEVAYWYILSIGAQTDFLSV |
| | | FFSGYTFKHKMVYEDTLTLFPFSGETVFMSMENPGLWILGCHN |
| | | SDFRNRGMTALLKVSSCDKNTGDYYEDSYEDISAYLLSKNNAI |
| | | EPRSFSQNSRHPSTRQKQFNATTIPENDIEKTDPWFAHRTPMPKI |
| | | QNVSSSDLLMLLRQSPTPHGLSLSDLQEAKYETFSDDPSPGAIDS |
| | | NNSLSEMTHFRPQLHHSGDMVFTPESGLQLRLNEKLGTTAATE |
| | | LKKLDFKVSSTSNNLISTIPSDNLAAGTDNTSSLGPPSMPVHYDS |
| | | QLDTTLFGKKSSPLTESGGPLSLSEENNDSKLLESGLMNSQESS |
| | | WGKNVSSTESGRLFKGKRAHGPALLTKDNALFKVSISLLKTNK |
| | | TSNNSATNRKTHIDGPSLLIENSPSVWQNILESDTEFKKVTPLIH |
| | | DRMLMDKNATALRLNHMSNKTTSSKNMEMVQQKKEGPIPPD |
| | | AQNPDMSFFKMLFLPESARWIQRTHGKNSLNSGQGPSPKQLVS |
| | | LGPEKSVEGQNFLSEKNKVVVGKGEFTKDVGLKEMVFPSSRNL |
| | | FLTNLDNLHENNTHNQEKKIQEEIEKKETLIQENVVLPQIHTVTG |
| | | TKNFMKNLFLLSTRQNVEGSYDGAYAPVLQDFRSLNDSTNRTK |
| | | KHTAHFSKKGEEENLEGLGNQTKQIVEKYACTTRISPNTSQQNF |
| | | VTQRSKRALKQFRLPLEETELEKRIIVDDTSTQWSKNMKHLTPS |
| | | TLTQIDYNEKEKGAITQSPLSDCLTRSHSIPQANRSPLPIAKVSSF |
| | | PSIRPIYLTRVLFQDNSSHLPAASYRKKDSGVQESSHFLQGAKK |
| | | NNLSLAILTLEMTGDQREVGSLGTSATNSVTYKKVENTVLPKP |
| | | DLPKTSGKVELLPKVHIYQKDLFPTETSNGSPGHLDLVEGSLLQ |
| | | GTEGAIKWNEANRPGKVPFLRVATESSAKTPSKLLDPLAWDNH |
| | | YGTQIPKEEWKSQEKSPEKTAFKKKDTILSLNACESNHAIAAINE |
| | | GQNKPEIEVTWAKQGRTERLCSQNPPVLKRHQREITRTTLQSDQ |
| | | EEIDYDDTISVEMKKEDFDIYDEDENQSPRSFQKKTRHYFIAAV |
| | | ERLWDYGMSSSPHVLRNRAQSGSVPQFKKVVFQEFTDGSFTQP |
| | | LYRGELNEHLGLLGPYIRAEVEDNIMVTFRNQASRPYSFYSSLIS |
| | | YEEDQRQGAEPRKNFVKPNETKTYFWKVQHHMAPTKDEFDCK |
| | | AWAYFSDVDLEKDVHSGLIGPLLVCHTNTLNPAHGRQVTVQEF |
| | | ALFFTIFDETKSWYFTENMERNCRAPCNIQMEDPTFKENYRFHA |
| | | INGYIMDTLPGLVMAQDQRIRWYLLSMGSNENIHSIHFSGHVFT |
| | | VRKKEEYKMALYNLYPGVFETVEMLPSKAGIWRVECLIGEHLH |
| | | AGMSTLFLVYSNKCQTPLGMASGHIRDFQITASGQYGQWAPKL |
| | | ARLHYSGSINAWSTKEPFSWIKVDLLAPMIIHGIKTQGARQKFSS |
| | | LYISQFIEVIYSLDGKKWQTYRGNSTGTLMVFFGNVDSSGIKHNI |
| | | FNPPIIARYIRLHPTHYSIRSTLRMELMGCDLNSCSMPLGMESKA |
| | | ISDAQITASSYFTNMFATWSPSKARLHLQGRSNAWRPQVNNPK |
| | | EWLQVDFQKTMKVTGVTTQGVKSLLTSMYVKEFLISSSQDGH |
| | | QWTLFFQNGKVKVFQGNQDSFTPVVNSLDPPLLTRYLRIHPQS |
| | | WVHQIALRMEVLGCEAQDLY |
| 5 | FVIII amino | MQIELSTCFFLCLLRFCFSATRRYYLGAVELSWDYMQSDLGELP |
| | acid | VDARFPPRVPKSFPFNTSVVYKKTLFVEFTDHLFNIAKPRPPWM |
| | sequence | GLLGPTIQAEVYDTVVITLKNMASHPVSLHAVGVSYWKASEGA |
| | comprising | EYDDQTSQREKEDDKVFPGGSHTYVWQVLKENGPMASDPLCL |
| | XTEN | TYSYLSHVDLVKDLNSGLIGALLVCREGSLAKEKTQTLHKFILL |
| | (XTEN in | FAVFDEGKSWHSETKNSLMQDRDAASARAWPKMHTVNGYVN |
| | bold and | RSLPGLIGCHRKSVYWHVIGMGTTPEVHSIFLEGHTFLVRNHRQ |
| | underline) | ASLEISPITFLTAQTLLMDLGQFLLFCHISSHQHDGMEAYVKVD |
| | | SCPEEPQLRMKNNEEAEDYDDDLTDSEMDVVRFDDDNSPSFIQI |
| | | RSVAKKHPKTWVHYIAAEEEDWDYAPLVLAPDDRSYKSQYLN |
| | | NGPQRIGRKYKKVRFMAYTDETFKTREAIQHESGILGPLLYGEV |
| | | GDTLLIIFKNQASRPYNIYPHGITDVRPLYSRRLPKGVKHLKDFP |
| | | ILPGEIFKYKWTVTVEDGPTKSDPRCLTRYYSSFVNMERDLASG |
| | | LIGPLLICYKESVDQRGNQIMSDKRNVILFSVFDENRSWYLTENI |
| | | QRFLPNPAGVQLEDPEFQASNIMHSINGYVFDSLQLSVCLHEVA |
| | | YWYILSIGAQTDFLSVFFSGYTFKHKMVYEDTLTLFPFSGETVF |
| | | MSMENPGLWILGCHNSDFRNRGMTALLKVSSCDKNTGDYYED |
| | | SYEDISAYLLSKNNAIEPRSFSQNTSESATPESGPGSEPATSGSE |
| | | TPGTSESATPESGPGSEPATSGSETPGTSESATPESGPGTSTE |
| | | PSEGSAPGSPAGSPTSTEEGTSESATPESGPGSEPATSGSETP |
| | | GTSESATPESGPGSPAGSPTSTEEGSPAGSPTSTEEGASSPPVL |
| | | KRHQREITRTTLQSDQEEIDYDDTISVEMKKEDFDIYDEDENQS |
| | | PRSFQKKTRHYFIAAVERLWDYGMSSSPHVLRNRAQSGSVPQF |
| | | KKVVFQEFTDGSFTQPLYRGELNEHLGLLGPYIRAEVEDNIMVT |
| | | FRNQASRPYSFYSSLISYEEDQRQGAEPRKNFVKPNETKTYFWK |
| | | VQHFIMAPTKDEFDCKAWAYFSDVDLEKDVHSGLIGPLLVCHT |
| | | NTLNPAHGRQVTVQEFALFFTIFDETKSWYFTENMERNCRAPC |
| | | NIQMEDPTFKENYRFHAINGYIMDTLPGLVMAQDQRIRWYLLS |
| | | MGSNENIHSIHFSGHVFTVRKKEEYKMALYNLYPGVFETVEML |
| | | PSKAGIWRVECLIGEHLHAGMSTLFLVYSNKCQTPLGMASGHI |
| | | RDFQITASGQYGQWAPKLARLHYSGSINAWSTKEPFSWIKVDL |
| | | LAPMIIHGIKTQGARQKFSSLYISQFIIMYSLDGKKWQTYRGNST |
| | | GTLMVFFGNVDSSGIKHNIFNPPIIARYIRLHPTHYSIRSTLRMEL |
| | | MGCDLNSCSMPLGMESKAISDAQITASSYFTNMFATWSPSKAR |
| | | LHLQGRSNAWRPQVNNPKEWLQVDFQKTMKVTGVTTQGVKS |
| | | LLTSMYVKEFLISSSQDGHQWTLFFQNGKVKVFQGNQDSFTPV |
| | | VNSLDPPLLTRYLRIHPQSWVHQIALRMEVLGCEAQDLY |
| 6 | FIX-R338L | MQRVNMIMAESPGLITICLLGYLLSAECTVFLDHENANKIL |
| | amino acid | NRPKRYNSGKLEEFVQGNLERECMEEKCSFEEAREVFENTERT |
| | sequence | TEFWKQYVDGDQCESNPCLNGGSCKDDINSYECWCPFGFEGK |
| | (signal | NCELDVTCNIKNGRCEQFCKNSADNKVVCSCTEGYRLAENQKS |
| | peptide in | CEPAVPFPCGRVSVSQTSKLTRAETVFPDVDYVNSTEAETILDNI |
| | bold and | TQSTQSFNDFTRVVGGEDAKPGQFPWQVVLNGKVDAFCGGSIV |
| | underline) | NEKWIVTAAHCVETGVKITVVAGEHNIEETEHTEQKRNVIRIIPH |
| | | HNYNAAINKYNHDIALLELDEPLVLNSYVTPICIADKEYTNIFLK |
| | | FGSGYVSGWGRVFHKGRSALVLQYLRVPLVDRATCLLSTKFTI |
| | | YNNMFCAGFHEGGRDSCQGDSGGPHVTEVEGTSFLTGIISWGE |
| | | ECAMKGKYGIYTKVSRYVNWIKEKTKLT |
| 7 | Target | tccataaagtaggaaacactaca |
| | sequence for | |
| | miR-142 | |

The lentiviral vectors of the present disclosure are therapeutically effective when administered at doses of 5×10¹⁰ TU/kg or lower, 10⁹ TU/kg or lower, or 10⁸ TU/kg or lower. At such dosages, the administration of the lentiviral vectors of the disclosure can result in an increase in plasma FVIII activity in a subject in need thereof at least about 2-fold, at least about 3-fold, at least about 4-fold, at least about 5-fold, at least about 6-fold, at least about 7-fold, at least about 8-fold, at least about 9-fold, at least about 10-fold, at least about 11-fold, at least about 12-fold, at least about 13-fold, at least about 14-fold, at least about 15-fold, at least about 20-fold, at least about 25-fold, at least about 30-fold, at least about 35-fold, at least about 40-fold, at least about 45-fold, at least about 50-fold, at least about 55-fold, at least about 60-fold, at least about 65-fold, at least about 70-fold, at least about 75-fold, at least about 80-fold, at least about 85-fold, at least about 90-fold, at least about 95-fold, at least about 100-fold, at least about 110-fold, at least about 120-fold, at least about 130-fold, at least about 140-fold, at least about 150-fold, at least about 160-fold, at least about 170-fold, at least about 180-fold, at least about 190-fold, or at least about 200-fold with respect to basal levels in the subject, relative to levels in a subject administered a control lentiviral vector, relative to levels in a subject administered a control nucleic acid molecule, or relative to levels in a subject after administration of a polypeptide encoded by the control nucleic acid molecule.

In certain embodiments, it will be useful to include within the lentiviral vector one or more miRNA target sequences which, for example, are operably linked to the transgene, such as the optimized FVIII transgene. Thus, the disclosure also provides at least one miRNA sequence target operably linked to the optimized FVIII or optimized FIX nucleotide sequence or otherwise inserted within a lentiviral vector. More than one copy of a miRNA target sequence included in the lentiviral vector can increase the effectiveness of the system.

Also included are different miRNA target sequences. For example, lentiviral vectors which express more than one transgene can have the transgene under control of more than one miRNA target sequence, which can be the same or different. The miRNA target sequences can be in tandem, but other arrangements are also included. The transgene expression cassette, containing miRNA target sequences, can also be inserted within the lentiviral vector in antisense orientation. Antisense orientation can be useful in the production of viral particles to avoid expression of gene products which can otherwise be toxic to the producer cells.

In other embodiments, the lentiviral vector comprises 1, 2, 3, 4, 5, 6, 7 or 8 copies of the same or different miRNA target sequence. In certain embodiments, the lentiviral vector does not include any miRNA target sequence. Choice of whether or not to include an miRNA target sequence (and how many) will be guided by known parameters such as the intended tissue target, the level of expression required, etc.

In one embodiment, the target sequence is an miR-223 target which has been reported to block expression most effectively in myeloid committed progenitors and at least partially in the more primitive HSPC. miR-223 target can block expression in differentiated myeloid cells including granulocytes, monocytes, macrophages, myeloid dendritic cells. miR-223 target can also be suitable for gene therapy applications relying on robust transgene expression in the lymphoid or erythroid lineage. miR-223 target can also block expression very effectively in human HSC.

In another embodiment, the target sequence is an miR142 target (tccataaagtaggaaacactaca (SEQ ID NO: 7)). In one embodiment, the lentiviral vector comprises 4 copies of miR-142 target sequences. In certain embodiments, the complementary sequence of hematopoietic-specific microRNAs, such as miR-142 (142T), is incorporated into the 3′ untranslated region of a lentiviral vector, making the transgene-encoding transcript susceptible to miRNA-mediated down-regulation. By this method, transgene expression can be prevented in hematopoietic-lineage antigen presenting cells (APC), while being maintained in non-hematopoietic cells (Brown et al., Nat Med 2006). This strategy can impose a stringent post-transcriptional control on transgene expression and thus enables stable delivery and long-term expression of transgenes. In some embodiments, miR-142 regulation prevents immune-mediated clearance of transduced cells and/or induce antigen-specific Regulatory T cells (T regs) and mediate robust immunological tolerance to the transgene-encoded antigen.

In some embodiments, the target sequence is an miR181 target. Chen C-Z and Lodish H, Seminars in Immunology (2005) 17(2):155-165 discloses miR-181, a miRNA specifically expressed in B cells within mouse bone marrow (Chen and Lodish, 2005). It also discloses that some human miRNAs are linked to leukemias.

The target sequence can be fully or partially complementary to the miRNA. The term “fully complementary” means that the target sequence has a nucleic acid sequence which is 100% complementary to the sequence of the miRNA which recognizes it. The term “partially complementary” means that the target sequence is only in part complementary to the sequence of the miRNA which recognizes it, whereby the partially complementary sequence is still recognized by the miRNA. In other words, a partially complementary target sequence in the context of the present disclosure is effective in recognizing the corresponding miRNA and effecting prevention or reduction of transgene expression in cells expressing that miRNA. Examples of the miRNA target sequences are described at WO2007/000668, WO2004/094642, WO2010/055413, or WO2010/125471, which are incorporated herein by reference in their entireties.

### A.2. Excipients, Carriers, and Other Constituents of Formulations

For purposes of gene therapy, lentiviral vectors (LVs) are often administered systemically, i.e., directly into the bloodstream of patients. Thus, it is of wide interest to create formulations of LVs that are not toxic, yet still maintain stability and potency of the LVs. When testing vehicles to create LV formulations suitable for systemic administration, certain core principles must be kept in mind. To ensure minimal shock to the subject to which the formulation is being administered, pH, ionic concentration, and osmolarity must be optimized to match physiological conditions. The combination of which buffers, salts, and carbohydrates (to regulate pH, ionic concentration, and osmolarity, respectively) is not a priori determinable and must be tested experimentally.

For example, US20170073702A1 discloses the TSSM vehicle (20 mM TRIS, 100 mM NaCl, 1% (w/v) Sucrose, 1% (w/v) Mannitol, pH 7.3), which could have been predicted to be non-toxic in systemic administration to mammals. However, it was found (see Example 2) that a formulation using TSSM alone or in combination with LVs was toxic to mice. Surprisingly, however, when a Phosphate vehicle (10 mM phosphate, 100 mM NaCl, 3% (w/v) sucrose, 0.05% (w/v) P188, pH 7.3) or Histidine vehicle (20 mM histidine, 100 mM NaCl, 3% (w/v) sucrose, 0.05% (w/v) P188, pH 6.5) was used, the formulation was not toxic to mice.

As would not have been predicted a priori, the formulation with the Phosphate vehicle resulted in higher stability and integrity of LVs as compared to the TSSM formulation (Example 3 and Example 4). Unexpectedly, the Histidine vehicle conferred greater stability on LVs than the Phosphate vehicle (Example 4). This key feature of the Histidine formulation, namely the compatibility of the lentiviral vector with the lower pH of the vehicle (pH 6.5) as compared to the neutral pH (pH 7.3) of phosphate or TRIS buffers, was surprising at least for the following reason. The VSV-G envelope protein is an important component of the lentiviral vector, which facilitates its infectivity into the cell. The pI (isoelectric point) of VSV-G is approx. 5, which means that as the pH of the solution nears the pI, the charge on the protein becomes more neutral. As proteins become more neutral in charge, their ability to attract one another is greater and this may lead to aggregation (degradation mechanism) thereby losing the ability to infect.

Also surprising was that the inclusion of a surfactant, poloxamer 188 (P188), in the Phosphate and Histidine formulations conferred increased LV stability and integrity as compared to the TSSM formulation. This was unexpected because surfactants would have been predicted to destabilize the outer lipid membrane envelope of the LVs, thus decreasing LV stability and integrity. Similarly, the removal of mannitol from the formulation would not have been a priori obvious to result in formulations with higher LV stability and integrity (Example 3 and Example 4).

As such, based on the findings described herein, a lentiviral vector preparation comprising a TRIS-free buffer system was found to provide increased lentiviral vector stability and integrity. It will be appreciated by those of skill in the art that a TRIS-free buffer system refers to any buffer system that does not comprise TRIS (also known as tris(hydroxymethyl)aminomethane, tromethamine, or THAM). In certain embodiments, the TRIS-free buffer system comprises phosphate. In certain embodiments, the TRIS-free buffer system comprises histidine.

In certain embodiments, the pH of the TRIS-free buffer system or of the preparation is from about 6.0 to about 8.0. In certain embodiments, the pH of the TRIS-free buffer system or of the preparation is from about 6.0 to about 7.5. In certain embodiments, the pH of the TRIS-free buffer system or of the preparation is from about 6.0 to about 7.0. In certain embodiments, the pH of the TRIS-free buffer system or of the preparation is from about 7.0 to about 8.0. In certain embodiments, the pH of the TRIS-free buffer system or of the preparation is about 6.5. In certain embodiments, the pH of the TRIS-free buffer system or of the preparation is about 7.3.

The skilled artisan will be able to determine a suitable buffer component to be employed in a TRIS-free buffer system of a preparation disclosed herein in order to maintain the target pH or target pH range. In certain embodiments, the TRIS-free buffer system comprises a buffer component having an effective pH buffering range of from about 6.0 to about 8.0. In certain embodiments, the TRIS-free buffer system comprises a buffer component having an effective pH buffering range of from about 6.0 to about 7.5. In certain embodiments, the TRIS-free buffer system comprises a buffer component having an effective pH buffering range of from about 6.0 to about 7.0. In certain embodiments, the TRIS-free buffer system comprises a buffer component having an effective pH buffering range of from about 7.0 to about 8.0. In certain embodiments, the TRIS-free buffer system comprises a buffer component having an effective pH buffering range that can maintain a pH of 6.5. In certain embodiments, the TRIS-free buffer system comprises a buffer component having an effective pH buffering range that can maintain a pH of 7.3.

Compositions containing a lentiviral gene therapy vector disclosed herein, or a host cell of the present disclosure (e.g., a hepatocyte targeted with a lentiviral gene therapy vector disclosed herein) can contain a suitable pharmaceutically acceptable carrier. For example, they can contain excipients and/or auxiliaries that facilitate processing of the active compounds into preparations designed for delivery to the site of action.

The pharmaceutical composition can be formulated for parenteral administration (i.e. intravenous, subcutaneous, or intramuscular) by bolus injection. Formulations for injection can be presented in unit dosage form, e.g., in ampoules or in multidose containers with an added preservative. The compositions can take such forms as suspensions, solutions, or emulsions in oily or aqueous vehicles, and contain formulatory agents such as suspending, stabilizing and/or dispersing agents. Alternatively, the active ingredient can be in powder form for constitution with a suitable vehicle, e.g., pyrogen free water.

Suitable formulations for parenteral administration also include aqueous solutions of the active compounds in water-soluble form, for example, water-soluble salts. In addition, suspensions of the active compounds as appropriate oily injection suspensions can be administered. Suitable lipophilic solvents or vehicles include fatty oils, for example, sesame oil, or synthetic fatty acid esters, for example, ethyl oleate or triglycerides. Aqueous injection suspensions can contain substances, which increase the viscosity of the suspension, including, for example, sodium carboxymethyl cellulose, sorbitol and dextran. Optionally, the suspension can also contain stabilizers. Liposomes also can be used to encapsulate the molecules of the disclosure for delivery into cells or interstitial spaces. Exemplary pharmaceutically acceptable carriers are physiologically compatible solvents, dispersion media, coatings, antibacterial and antifungal agents, isotonic and absorption delaying agents, water, saline, phosphate buffered saline, dextrose, glycerol, ethanol and the like. In some embodiments, the composition comprises isotonic agents, for example, sugars, polyalcohols such as mannitol, sorbitol, or sodium chloride. In other embodiments, the compositions comprise pharmaceutically acceptable substances such as wetting agents or minor amounts of auxiliary substances such as wetting or emulsifying agents, preservatives or buffers, which enhance the shelf life or effectiveness of the active ingredients.

Compositions of the disclosure can be in a variety of forms, including, for example, liquid (e.g., injectable and infusible solutions), dispersions, suspensions, semi-solid and solid dosage forms. The preferred form depends on the mode of administration and therapeutic application.

The composition can be formulated as a solution, micro emulsion, dispersion, liposome, or other ordered structure suitable to high drug concentration. Sterile injectable solutions can be prepared by incorporating the active ingredient in the required amount in an appropriate solvent with one or a combination of ingredients enumerated above, as required, followed by filtered sterilization. Generally, dispersions are prepared by incorporating the active ingredient into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

The active ingredient can be formulated with a controlled-release formulation or device. Examples of such formulations and devices include implants, transdermal patches, and microencapsulated delivery systems. Biodegradable, biocompatible polymers can be used, for example, ethylene vinyl acetate, polyanhydrides, polyglycolic acid, collagen, polyorthoesters, and polylactic acid. Methods for the preparation of such formulations and devices are known in the art. See, e.g., Sustained and Controlled Release Drug Delivery Systems, J. R. Robinson, ed., Marcel Dekker, Inc., New York, 1978.

Injectable depot formulations can be made by forming microencapsulated matrices of the drug in biodegradable polymers such as polylactide-polyglycolide. Depending on the ratio of drug to polymer, and the nature of the polymer employed, the rate of drug release can be controlled. Other exemplary biodegradable polymers are polyorthoesters and polyanhydrides. Depot injectable formulations also can be prepared by entrapping the drug in liposomes or microemulsions.

Supplementary active compounds can be incorporated into the compositions. In one embodiment, the chimeric protein of the disclosure is formulated with another clotting factor, or a variant, fragment, analogue, or derivative thereof. For example, the clotting factor includes, but is not limited to, factor V, factor VII, factor VIII, factor IX, factor X, factor XI, factor XII, factor XIII, prothrombin, fibrinogen, von Willebrand factor or recombinant soluble tissue factor (rsTF) or activated forms of any of the preceding. The clotting factor of hemostatic agent can also include anti-fibrinolytic drugs, e.g., epsilon-amino-caproic acid, tranexamic acid.

Dosage regimens can be adjusted to provide the optimum desired response. For example, a single bolus can be administered, several divided doses can be administered over time, or the dose can be proportionally reduced or increased as indicated by the exigencies of the therapeutic situation. It is advantageous to formulate parenteral compositions in dosage unit form for ease of administration and uniformity of dosage. See, e.g., Remington's Pharmaceutical Sciences (Mack Pub. Co., Easton, Pa. 1980).

In addition to the active compound, the liquid dosage form can contain inert ingredients such as water, ethyl alcohol, ethyl carbonate, ethyl acetate, benzyl alcohol, benzyl benzoate, propylene glycol, 1,3-butylene glycol, dimethylformamide, oils, glycerol, tetrahydrofurfuryl alcohol, polyethylene glycols, and fatty acid esters of sorbitan.

Non-limiting examples of suitable pharmaceutical carriers are also described in Remington's Pharmaceutical Sciences by E. W. Martin. Some examples of excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. The composition can also contain pH buffering reagents, and wetting or emulsifying agents.

For oral administration, the pharmaceutical composition can take the form of tablets or capsules prepared by conventional means. The composition can also be prepared as a liquid for example a syrup or a suspension. The liquid can include suspending agents (e.g., sorbitol syrup, cellulose derivatives or hydrogenated edible fats), emulsifying agents (lecithin or acacia), non-aqueous vehicles (e.g., almond oil, oily esters, ethyl alcohol, or fractionated vegetable oils), and preservatives (e.g., methyl or propyl-p-hydroxybenzoates or sorbic acid). The preparations can also include flavoring, coloring and sweetening agents. Alternatively, the composition can be presented as a dry product for constitution with water or another suitable vehicle.

For buccal administration, the composition can take the form of tablets or lozenges according to conventional protocols.

For administration by inhalation, the compounds for use according to the present disclosure are conveniently delivered in the form of a nebulized aerosol with or without excipients or in the form of an aerosol spray from a pressurized pack or nebulizer, with optionally a propellant, e.g., dichlorodifluoromethane, trichlorofluoromethane, dichlorotetrafluoromethane, carbon dioxide or other suitable gas. In the case of a pressurized aerosol the dosage unit can be determined by providing a valve to deliver a metered amount. Capsules and cartridges of, e.g., gelatin for use in an inhaler or insufflator can be formulated containing a powder mix of the compound and a suitable powder base such as lactose or starch.

The pharmaceutical composition can also be formulated for rectal administration as a suppository or retention enema, e.g., containing conventional suppository bases such as cocoa butter or other glycerides.

In one embodiment, the pharmaceutical composition comprises a lentiviral vector comprising an optimized nucleic acid molecule encoding a polypeptide having Factor VIII or Factor IX activity, and a pharmaceutically acceptable carrier. In another embodiment, the pharmaceutical composition comprises a host cell (e.g., a hepatocyte) comprising a lentiviral vector comprising an optimized nucleic acid molecule encoding a polypeptide having Factor VIII or Factor IX activity, and a pharmaceutically acceptable carrier.

In some embodiments, the composition is administered by a route selected from the group consisting of topical administration, intraocular administration, parenteral administration, intrathecal administration, subdural administration and oral administration. The parenteral administration can be intravenous or subcutaneous administration.

A fundamental aspect for ensuring the transition of therapeutic formulations from the lab into manufacturable and marketable products of high and consistent quality is their stability in the dosage form. Owing to their complex chemistry and structure, proteins, such as surface and capsid proteins of viruses, are susceptible to various forms of physical and chemical degradation that can compromise the biological efficacy and safety of the final drug product. Protein aggregation for example is a key quality attribute that is routinely monitored for protein-based products and is critical to the determination of product shelf life. At a fundamental level, protein aggregation is linked to the stability of the native form of the protein, with a growth in non-native cell (e.g., a non-native mammalian cell) generally linked to an increased rate and extent of aggregation. Thus, it is no surprise that attempts to control and minimize aggregation during product shelf life (kinetic stability) are often mediated through the use of excipients or formulation conditions intended to increase conformational stability of the protein. Essentially, the intent is to stabilize the protein in its native conformation in order to minimize the population of aggregation-competent “non-native” species. Sugars and polyols, such as sucrose, trehalose, mannitol, sorbitol etc. are often used to stabilize proteins in their native state and reduce rates of aggregation. However, an unwanted effect of using these stabilizers is the concentration-dependent increase in solution viscosity.

Solution viscosity is a key attribute of protein products especially those that are formulated at high protein concentrations and it can critically impact the utility and success of the product. The manufacturability of a product and the end use by the patient or healthcare practitioner is intimately linked to the ability of a solution to flow seamlessly. High viscosity, for example, can necessitate the use of specialized administration devices or protocols which may not always be suitable for the desired population thereby limiting the use of the product. In other instances, high solution viscosity may require the application of manufacturing technologies which may negatively impact the stability of the protein (for example high-temperature processing). It is thus not unusual to employ viscosity-reducing excipients, such as salts and amino acids, in high protein concentration solutions. However, these excipients can negatively impact the stability of the protein thereby resulting in solutions with an increased aggregation rate compared to high-viscosity control solutions lacking the viscosity-reducing agent. In essence, commonly employed stabilizers and the viscosity-reducing excipients can have an opposite effect on product performance thereby complicating its development.

Another critical attribute for injectable products (most protein-based products) that needs to be considered is its osmolality. While intravenous solutions generally need to be isotonic, it is not unusual for subcutaneous solutions to be hypertonic. In fact, there is evidence in literature of hypertonic formulations resulting in enhanced protein bioavailability following subcutaneous administration (Fathallah, A. M. et al, Biopharm Drug Dispos. 2015 March; 36(2):115-25). Thus, the impact of solution osmolality (and thus tonicity) on injection site discomfort and/or reaction as well as bioavailability in the patient population needs to be carefully monitored and characterized during clinical development phases.

Formulations may sometimes contain surfactants, such as poloxamers and polysorbates, which may confer certain benefits. Poloxamers are non-ionic poly (ethylene oxide) (PEO)-poly (propylene oxide) (PPO) copolymers. They are used in pharmaceutical formulations as surfactants, emulsifying agents, solubilizing agents, dispersing agents, and in vivo absorbance enhances. Poloxamers are synthetic triblock copolymers with the following core formula: (PEO)a-(PPO)b-(PEO)a. All poloxamers have similar chemical structures but with different molecular weights and composition of the hydrophilic PEO block and hydrophobic PPO block. Two of the most commonly used poloxamers are poloxamer 188 (a=80, b=27) with molecular weight ranging from 7680 to 9510 Da, and poloxamer 407 (a=101, b=56) with molecular weight ranging from 9840 to 14600 Da. Other poloxamers include: poloxamer 101 (P101), poloxamer 105 (P105), poloxamer 108 (P108), poloxamer 122 (P122), poloxamer 123 (P123), poloxamer 124 (P124), poloxamer 181 (P181), poloxamer 182 (P182), poloxamer 183 (P183), poloxamer 184 (P184), poloxamer 185 (P185), poloxamer 212 (P212), poloxamer 215 (P215), poloxamer 217 (P217), poloxamer 231 (P231), poloxamer 234 (P234), poloxamer 235 (P235), poloxamer 237 (P237), poloxamer 238 (P238), poloxamer 282 (P282), poloxamer 284 (P284), poloxamer 288 (P288), poloxamer 331 (P331), poloxamer 333 (P333), poloxamer 334 (P334), poloxamer 335 (P335), poloxamer 338 (P338), poloxamer 401 (P401), poloxamer 402 (P402), and poloxamer 403 (P403).

Polysorbates are a class of emulsifiers used in some pharmaceutical and food preparation formulations. Polysorbates are oily liquids derived from ethoxylated sorbitan, a derivative of sorbitol, esterified with fatty acids. Common brand names for polysorbates include Scattics, Alkest, Canarcel, and Tween. The naming convention for polysorbates usually follows: polysorbate x (polyoxyethylene (y) sorbitan mono‘z’), where x is related to the type of fatty acid (z) associated with the polyoxyethylene sorbitan and y refers to the total number of oxyethylene —(CH₂CH₂O)— groups found in the polysorbate molecule. Examples of polysorbates include: (a) polysorbate 20 (polyoxyethylene (20) sorbitan monolaurate), (b) polysorbate 40 (polyoxyethylene (20) sorbitan monopalmitate), (c) polysorbate 60 (polyoxyethylene (20) sorbitan monostearate), and (d) polysorbate 80 (polyoxyethylene (20) sorbitan monooleate).

### B. Lentiviral Vector (LV) Formulations for Use in Treating Blood Disorders

In one aspect, the present invention is directed to a recombinant lentiviral vector preparation comprising: (a) an effective dose of a recombinant lentiviral vector; (b) a TRIS-free buffer system; (c) a salt; (d) a surfactant; (e) a carbohydrate, and (f) a nucleotide sequence at least 80% identical to the Factor VIII (FVIII) coding sequence set forth in SEQ ID NO: 1 or SEQ ID NO: 2 or the Factor IX (FIX) coding sequence set forth in SEQ ID NO: 3, wherein the pharmaceutical composition is suitable for systemic administration to a human patient. In certain embodiments, the vector comprises the Factor VIII (FVIII) coding sequence set forth in SEQ ID NO: 1 or SEQ ID NO: 2. In certain embodiments, the vector comprises the Factor IX (FIX) coding sequence set forth in SEQ ID NO: 3.

In another aspect, the present invention provides a recombinant lentiviral vector preparation comprising: (a) a therapeutically effective dose of a recombinant lentiviral vector; (b) a TRIS-free buffer system; (c) a salt; (d) a surfactant; and (e) a carbohydrate, wherein the recombinant lentiviral vector comprises a nucleic acid comprising a nucleotide sequence at least 80%, at least 85%, at least 90%, at least 95%, or at least 99% identical to a Factor VIII (FVIII) coding sequence set forth in SEQ ID NO: 1 or SEQ ID NO: 2, or a nucleotide sequence at least 80%, at least 85%, at least 90%, at least 95%, or at least 99% identical to the Factor IX (FIX) coding sequence set forth in SEQ ID NO: 3, and wherein the pharmaceutical composition is suitable for systemic administration to a human patient. In certain embodiments, the recombinant lentiviral vector comprises a nucleic acid comprising a Factor VIII (FVIII) coding sequence set forth in SEQ ID NO: 1 or SEQ ID NO: 2. In certain embodiments, the recombinant lentiviral vector comprises a nucleic acid comprising the Factor IX (FIX) coding sequence set forth in SEQ ID NO: 3. In certain embodiments, the recombinant lentiviral vector comprises a nucleic acid consisting of a Factor VIII (FVIII) coding sequence set forth in SEQ ID NO: 1 or SEQ ID NO: 2. In certain embodiments, the recombinant lentiviral vector comprises a nucleic acid consisting of the Factor IX (FIX) coding sequence set forth in SEQ ID NO: 3.

In certain embodiments, the pH of the buffer system is from about 6.0 to about 8.0. In certain embodiments, the pH of the buffer system is from about 6.0 to about 7.5. In certain embodiments, the pH of the buffer system is from about 6.0 to about 7.0. In certain embodiments, the pH of the buffer system is from about 6.0 to about 8.0. In certain embodiments, the pH of the buffer system is about 6.5. In certain embodiments, the pH of the buffer system is about 7.3. In certain embodiments, the buffer system is a phosphate buffer or a histidine buffer. In certain embodiments, the concentration of the phosphate or histidine buffer is from about 5 mM to about 30 mM. In certain embodiments, the concentration of the phosphate buffer is from about 10 mM to about 20 mM, from about 10 mM to about 15 mM, from about 20 mM to about 30 mM, from about 20 mM to about 25 mM, or from about 15 mM to about 20 mM. In certain embodiments, the salt is a chloride salt. In certain embodiments, the concentration of the chloride salt is from about 80 mM to about 150 mM. In certain embodiments, the concentration of the salt is about 100 mM, about 110 mM, about 130 mM, or about 150 mM. In certain embodiments, the surfactant is a poloxamer or a polysorbate. In certain embodiments, the concentration of the poloxamer or polysorbate is from about 0.01% (w/v) to about 0.1% (w/v). In certain embodiments, the carbohydrate is sucrose. In certain embodiments, the concentration of the carbohydrate is from about 0.5% (w/v) to about 5% (w/v). In certain embodiments, the chloride salt is sodium chloride (NaCl). In certain embodiments, the poloxamer is selected from the group consisting of poloxamer 101 (P101), poloxamer 105 (P105), poloxamer 108 (P108), poloxamer 122 (P122), poloxamer 123 (P123), poloxamer 124 (P124), poloxamer 181 (P181), poloxamer 182 (P182), poloxamer 183 (P183), poloxamer 184 (P184), poloxamer 185 (P185), poloxamer 188 (P188), poloxamer 212 (P212), poloxamer 215 (P215), poloxamer 217 (P217), poloxamer 231 (P231), poloxamer 234 (P234), poloxamer 235 (P235), poloxamer 237 (P237), poloxamer 238 (P238), poloxamer 282 (P282), poloxamer 284 (P284), poloxamer 288 (P288), poloxamer 331 (P331), poloxamer 333 (P333), poloxamer 334 (P334), poloxamer 335 (P335), poloxamer 338 (P338), poloxamer 401 (P401), poloxamer 402 (P402), poloxamer 403 (P403), poloxamer 407 (P407), and a combination thereof. In certain embodiments, the polysorbate is selected from the group consisting of polysorbate 20, polysorbate 40, polysorbate 60, polysorbate 80, and a combination thereof. In certain embodiments, the pH of the phosphate or histidine buffer is about 6.1, about 6.3, about 6.5, about 6.7, about 6.9, about 7.1, about 7.3, about 7.5, about 7.7, or about 7.9. In certain embodiments, the concentration of the phosphate or histidine buffer is about 10 mM, about 15 mM, about 20 mM, or about 25 mM. In certain embodiments, the chloride salt is about 100 mM, about 110 mM, about 130 mM, or about 150 mM. In certain embodiments, the concentration of the poloxamer or polysorbate is about 0.03% (w/v), about 0.05% (w/v), about 0.07% (w/v), or about 0.09% (w/v). In certain embodiments, the concentration of the carbohydrate is about 1% (w/v), about 2% (w/v), about 3% (w/v), or about 4% (w/v). In certain embodiments, the poloxamer is poloxamer 188 (P188). In certain embodiments, the poloxamer is poloxamer 407 (P407).

In certain embodiments, the present disclosure provides a recombinant lentiviral vector preparation comprising: (a) a therapeutically effective dose of a recombinant lentiviral vector; (b) about 10 mM phosphate; (c) about 100 mM sodium chloride; (d) about 0.05% (w/v) poloxamer 188; and (e) about 3% (w/v) sucrose, wherein the pH of the preparation is about 7.3, wherein the pharmaceutical composition is suitable for systemic administration to a human patient, and wherein the recombinant lentiviral vector comprises a nucleic acid comprising a nucleotide sequence at least 80%, at least 85%, at least 90%, at least 95%, or at least 99% identical to a Factor VIII (FVIII) coding sequence set forth in SEQ ID NO: 1 or SEQ ID NO: 2, or a nucleotide sequence at least 80%, at least 85%, at least 90%, at least 95%, or at least 99% identical to the Factor IX (FIX) coding sequence set forth in SEQ ID NO: 3, and wherein the pharmaceutical composition is suitable for systemic administration to a human patient. In certain embodiments, the recombinant lentiviral vector comprises a nucleic acid comprising a Factor VIII (FVIII) coding sequence set forth in SEQ ID NO: 1 or SEQ ID NO: 2. In certain embodiments, the recombinant lentiviral vector comprises a nucleic acid comprising the Factor IX (FIX) coding sequence set forth in SEQ ID NO: 3. In certain embodiments, the recombinant lentiviral vector comprises a nucleic acid consisting of a Factor VIII (FVIII) coding sequence set forth in SEQ ID NO: 1 or SEQ ID NO: 2. In certain embodiments, the recombinant lentiviral vector comprises a nucleic acid consisting of the Factor IX (FIX) coding sequence set forth in SEQ ID NO: 3.

In certain embodiments, the present disclosure provides a recombinant lentiviral vector preparation comprising: (a) a therapeutically effective dose of a recombinant lentiviral vector; (b) about 10 mM phosphate; (c) about 130 mM sodium chloride; (d) about 0.05% (w/v) poloxamer 188; and (e) about 1% (w/v) sucrose, wherein the pH of the preparation is about 7.3, wherein the pharmaceutical composition is suitable for systemic administration to a human patient, and wherein the recombinant lentiviral vector comprises a nucleic acid comprising a nucleotide sequence at least 80%, at least 85%, at least 90%, at least 95%, or at least 99% identical to a Factor VIII (FVIII) coding sequence set forth in SEQ ID NO: 1 or SEQ ID NO: 2, or a nucleotide sequence at least 80%, at least 85%, at least 90%, at least 95%, or at least 99% identical to the Factor IX (FIX) coding sequence set forth in SEQ ID NO: 3, and wherein the pharmaceutical composition is suitable for systemic administration to a human patient. In certain embodiments, the recombinant lentiviral vector comprises a nucleic acid comprising a Factor VIII (FVIII) coding sequence set forth in SEQ ID NO: 1 or SEQ ID NO: 2. In certain embodiments, the recombinant lentiviral vector comprises a nucleic acid comprising the Factor IX (FIX) coding sequence set forth in SEQ ID NO: 3. In certain embodiments, the recombinant lentiviral vector comprises a nucleic acid consisting of a Factor VIII (FVIII) coding sequence set forth in SEQ ID NO: 1 or SEQ ID NO: 2. In certain embodiments, the recombinant lentiviral vector comprises a nucleic acid consisting of the Factor IX (FIX) coding sequence set forth in SEQ ID NO: 3.

In certain embodiments, the present disclosure provides a recombinant lentiviral vector preparation comprising: (a) a therapeutically effective dose of a recombinant lentiviral vector; (b) about 20 mM histidine; (c) about 100 mM sodium chloride; (d) about 0.05% (w/v) poloxamer 188; and (e) about 3% (w/v) sucrose, wherein the pH of the preparation is about 6.5, wherein the pharmaceutical composition is suitable for systemic administration to a human patient, and wherein the recombinant lentiviral vector comprises a nucleic acid comprising a nucleotide sequence at least 80%, at least 85%, at least 90%, at least 95%, or at least 99% identical to a Factor VIII (FVIII) coding sequence set forth in SEQ ID NO: 1 or SEQ ID NO: 2, or a nucleotide sequence at least 80%, at least 85%, at least 90%, at least 95%, or at least 99% identical to the Factor IX (FIX) coding sequence set forth in SEQ ID NO: 3, and wherein the pharmaceutical composition is suitable for systemic administration to a human patient. In certain embodiments, the recombinant lentiviral vector comprises a nucleic acid comprising a Factor VIII (FVIII) coding sequence set forth in SEQ ID NO: 1 or SEQ ID NO: 2. In certain embodiments, the recombinant lentiviral vector comprises a nucleic acid comprising the Factor IX (FIX) coding sequence set forth in SEQ ID NO: 3. In certain embodiments, the recombinant lentiviral vector comprises a nucleic acid consisting of a Factor VIII (FVIII) coding sequence set forth in SEQ ID NO: 1 or SEQ ID NO: 2. In certain embodiments, the recombinant lentiviral vector comprises a nucleic acid consisting of the Factor IX (FIX) coding sequence set forth in SEQ ID NO: 3.

In certain embodiments, the present disclosure provides a recombinant lentiviral vector preparation comprising: (a) a therapeutically effective dose of a recombinant lentiviral vector; (b) about 10 mM phosphate; (c) about 100 mM sodium chloride; (d) about 0.05% (w/v) poloxamer 188; and (e) about 3% (w/v) sucrose, wherein the pH of the preparation is about 7.0, wherein the pharmaceutical composition is suitable for systemic administration to a human patient, and wherein the recombinant lentiviral vector comprises a nucleic acid comprising a nucleotide sequence at least 80%, at least 85%, at least 90%, at least 95%, or at least 99% identical to a Factor VIII (FVIII) coding sequence set forth in SEQ ID NO: 1 or SEQ ID NO: 2, or a nucleotide sequence at least 80%, at least 85%, at least 90%, at least 95%, or at least 99% identical to the Factor IX (FIX) coding sequence set forth in SEQ ID NO: 3, and wherein the pharmaceutical composition is suitable for systemic administration to a human patient. In certain embodiments, the recombinant lentiviral vector comprises a nucleic acid comprising a Factor VIII (FVIII) coding sequence set forth in SEQ ID NO: 1 or SEQ ID NO: 2. In certain embodiments, the recombinant lentiviral vector comprises a nucleic acid comprising the Factor IX (FIX) coding sequence set forth in SEQ ID NO: 3. In certain embodiments, the recombinant lentiviral vector comprises a nucleic acid consisting of a Factor VIII (FVIII) coding sequence set forth in SEQ ID NO: 1 or SEQ ID NO: 2. In certain embodiments, the recombinant lentiviral vector comprises a nucleic acid consisting of the Factor IX (FIX) coding sequence set forth in SEQ ID NO: 3.

In certain embodiments, the present disclosure provides a recombinant lentiviral vector preparation comprising: (a) a therapeutically effective dose of a recombinant lentiviral vector; (b) about 20 mM histidine; (c) about 100 mM sodium chloride; (d) about 0.05% (w/v) poloxamer 188; and (e) about 3% (w/v) sucrose, wherein the pH of the preparation is about 7.0, wherein the pharmaceutical composition is suitable for systemic administration to a human patient, and wherein the recombinant lentiviral vector comprises a nucleic acid comprising a nucleotide sequence at least 80%, at least 85%, at least 90%, at least 95%, or at least 99% identical to a Factor VIII (FVIII) coding sequence set forth in SEQ ID NO: 1 or SEQ ID NO: 2, or a nucleotide sequence at least 80%, at least 85%, at least 90%, at least 95%, or at least 99% identical to the Factor IX (FIX) coding sequence set forth in SEQ ID NO: 3, and wherein the pharmaceutical composition is suitable for systemic administration to a human patient. In certain embodiments, the recombinant lentiviral vector comprises a nucleic acid comprising a Factor VIII (FVIII) coding sequence set forth in SEQ ID NO: 1 or SEQ ID NO: 2. In certain embodiments, the recombinant lentiviral vector comprises a nucleic acid comprising the Factor IX (FIX) coding sequence set forth in SEQ ID NO: 3. In certain embodiments, the recombinant lentiviral vector comprises a nucleic acid consisting of a Factor VIII (FVIII) coding sequence set forth in SEQ ID NO: 1 or SEQ ID NO: 2. In certain embodiments, the recombinant lentiviral vector comprises a nucleic acid consisting of the Factor IX (FIX) coding sequence set forth in SEQ ID NO: 3.

In certain embodiments, the recombinant lentiviral vector comprises a nucleotide sequence encoding VSV-G or a fragment thereof. In certain embodiments, the recombinant lentiviral vector comprises an enhanced transthyretin (ET) promoter. In certain embodiments, the recombinant lentiviral vector comprises a nucleotide sequence at least 90% identical to the target sequence for miR-142 set forth in SEQ ID NO: 7.

In certain embodiments, the recombinant lentiviral vector is isolated from a transfected host cell, including a CHO cell, a HEK293 cell, a BHK21 cell, a PER.C6 cell, an NSO cell, and a CAP cell. In certain embodiments, the host cell is a CD47-positive host cell.

In certain embodiments, the preparation is administered systemically to the human patient. In certain embodiments, the preparation is administered intravenously.

In certain embodiments, the pH of the buffer system is between 6.0 and 8.0. In certain embodiments, the buffer system is a phosphate buffer or a histidine buffer. In certain embodiments, the concentration of the phosphate or histidine buffer is between 5 mM and 30 mM. In certain embodiments, the concentration of the phosphate buffer is about 10 to about 20 mM, about 10 to about 15 mM, about 20 to about 30 mM, about 20 to about 25 mM, or about 15 to about 20 mM. In certain embodiments, the salt is a chloride salt. In certain embodiments, the concentration of the chloride salt is between 80 mM and 150 mM. In certain embodiments, the concentration of the salt is about 100 mM, about 110 mM, about 130 mM, or about 150 mM. In certain embodiments, the surfactant is a poloxamer or a polysorbate. In certain embodiments, the concentration of the poloxamer or polysorbate is between 0.01% (w/v) and 0.1% (w/v). In certain embodiments, the carbohydrate is sucrose. In certain embodiments, the concentration of the carbohydrate is between 0.5% (w/v) and 5% (w/v). In certain embodiments, the chloride salt is NaCl. In certain embodiments, the poloxamer is selected from the group consisting of poloxamer 101 (P101), poloxamer 105 (P105), poloxamer 108 (P108), poloxamer 122 (P122), poloxamer 123 (P123), poloxamer 124 (P124), poloxamer 181 (P181), poloxamer 182 (P182), poloxamer 183 (P183), poloxamer 184 (P184), poloxamer 185 (P185), poloxamer 188 (P188), poloxamer 212 (P212), poloxamer 215 (P215), poloxamer 217 (P217), poloxamer 231 (P231), poloxamer 234 (P234), poloxamer 235 (P235), poloxamer 237 (P237), poloxamer 238 (P238), poloxamer 282 (P282), poloxamer 284 (P284), poloxamer 288 (P288), poloxamer 331 (P331), poloxamer 333 (P333), poloxamer 334 (P334), poloxamer 335 (P335), poloxamer 338 (P338), poloxamer 401 (P401), poloxamer 402 (P402), poloxamer 403 (P403), poloxamer 407 (P407), and a combination thereof. In certain embodiments, the polysorbate is selected from the group consisting of polysorbate 20, polysorbate 40, polysorbate 60, polysorbate 80, and a combination thereof. In certain embodiments, the pH of the phosphate or histidine buffer is 6.1, 6.3, 6.5, 6.7, 6.9, 7.1, 7.3, 7.5, 7.7, or 7.9. In certain embodiments, the concentration of the phosphate or histidine buffer is 10 mM, 15 mM, 20 mM, or 25 mM. In certain embodiments, the chloride salt is 100 mM, 110 mM, 130 mM, or 150 mM. In certain embodiments, the concentration of the poloxamer or polysorbate is 0.03% (w/v), 0.05% (w/v), 0.07% (w/v), or 0.09% (w/v). In certain embodiments, the concentration of the carbohydrate is 1% (w/v), 2% (w/v), 3% (w/v), or 4% (w/v). In certain embodiments, the poloxamer is poloxamer 188 (P188). In certain embodiments, the poloxamer is poloxamer 407 (P407).

In one aspect, the present invention is directed to a recombinant lentiviral vector preparation comprising: (a) an effective dose of a recombinant lentiviral vector; (b) a TRIS-free buffer system; (c) a salt; (d) a surfactant; (e) a carbohydrate, and (f) an enhanced transthyretin (ET) promoter, wherein the pharmaceutical composition is suitable for systemic administration to a human patient. In certain embodiments, the vector further comprises a nucleotide sequence at least 80% identical to the Factor VIII (FVIII) coding sequence set forth in SEQ ID NO: 1 or SEQ ID NO: 2 or the Factor IX (FIX) coding sequence set forth in SEQ ID NO: 3, wherein the pharmaceutical composition is suitable for systemic administration to a human patient. In certain embodiments, the vector comprises the Factor VIII (FVIII) coding sequence set forth in SEQ ID NO: 1 or SEQ ID NO: 2. In certain embodiments, the vector comprises the Factor IX (FIX) coding sequence set forth in SEQ ID NO: 3.

In certain embodiments, the vector further comprises a nucleotide sequence encoding VSV-G or a fragment thereof. In certain embodiments, the pH of the buffer system is between 6.0 and 8.0. In certain embodiments, the buffer system is a phosphate buffer or a histidine buffer. In certain embodiments, the concentration of the phosphate or histidine buffer is between 5 mM and 30 mM. In certain embodiments, the concentration of the phosphate buffer is about 10 to about 20 mM, about 10 to about 15 mM, about 20 to about 30 mM, about 20 to about 25 mM, or about 15 to about 20 mM. In certain embodiments, the salt is a chloride salt. In certain embodiments, the concentration of the chloride salt is between 80 mM and 150 mM. In certain embodiments, the concentration of the salt is about 100 mM, about 110 mM, about 130 mM, or about 150 mM. In certain embodiments, the surfactant is a poloxamer or a polysorbate. In certain embodiments, the concentration of the poloxamer or polysorbate is between 0.01% (w/v) and 0.1% (w/v). In certain embodiments, the carbohydrate is sucrose. In certain embodiments, the concentration of the carbohydrate is between 0.5% (w/v) and 5% (w/v). In certain embodiments, the chloride salt is NaCl. In certain embodiments, the poloxamer is selected from the group consisting of poloxamer 101 (P101), poloxamer 105 (P105), poloxamer 108 (P108), poloxamer 122 (P122), poloxamer 123 (P123), poloxamer 124 (P124), poloxamer 181 (P181), poloxamer 182 (P182), poloxamer 183 (P183), poloxamer 184 (P184), poloxamer 185 (P185), poloxamer 188 (P188), poloxamer 212 (P212), poloxamer 215 (P215), poloxamer 217 (P217), poloxamer 231 (P231), poloxamer 234 (P234), poloxamer 235 (P235), poloxamer 237 (P237), poloxamer 238 (P238), poloxamer 282 (P282), poloxamer 284 (P284), poloxamer 288 (P288), poloxamer 331 (P331), poloxamer 333 (P333), poloxamer 334 (P334), poloxamer 335 (P335), poloxamer 338 (P338), poloxamer 401 (P401), poloxamer 402 (P402), poloxamer 403 (P403), poloxamer 407 (P407), and a combination thereof. In certain embodiments, the polysorbate is selected from the group consisting of polysorbate 20, polysorbate 40, polysorbate 60, polysorbate 80, and a combination thereof. In certain embodiments, the pH of the phosphate or histidine buffer is 6.1, 6.3, 6.5, 6.7, 6.9, 7.1, 7.3, 7.5, 7.7, or 7.9. In certain embodiments, the concentration of the phosphate or histidine buffer is 10 mM, 15 mM, 20 mM, or 25 mM. In certain embodiments, the chloride salt is 100 mM, 110 mM, 130 mM, or 150 mM. In certain embodiments, the concentration of the poloxamer or polysorbate is 0.03% (w/v), 0.05% (w/v), 0.07% (w/v), or 0.09% (w/v). In certain embodiments, the concentration of the carbohydrate is 1% (w/v), 2% (w/v), 3% (w/v), or 4% (w/v). In certain embodiments, the poloxamer is poloxamer 188 (P188). In certain embodiments, the poloxamer is poloxamer 407 (P407).In one aspect, the present invention is directed to a recombinant lentiviral vector preparation comprising: (a) an effective dose of a recombinant lentiviral vector; (b) a TRIS-free buffer system; (c) a salt; (d) a surfactant; (e) a carbohydrate, and (f) a nucleotide sequence at least 90% identical to the target sequence for miR-142 set forth in SEQ ID NO: 7, wherein the pharmaceutical composition is suitable for systemic administration to a human patient. In certain embodiments, the vector further comprises an enhanced transthyretin (ET) promoter. In certain embodiments, the vector further comprises a nucleotide sequence at least 80% identical to the Factor VIII (FVIII) coding sequence set forth in SEQ ID NO: 1 or SEQ ID NO: 2 or the Factor IX (FIX) coding sequence set forth in SEQ ID NO: 3. In certain embodiments, the vector comprises the Factor VIII (FVIII) coding sequence set forth in SEQ ID NO: 1 or SEQ ID NO: 2. In certain embodiments, the vector comprises the Factor IX (FIX) coding sequence set forth in SEQ ID NO: 3.

In certain embodiments, the vector further comprises a nucleotide sequence encoding VSV-G or a fragment thereof. In certain embodiments, the pH of the buffer system is between 6.0 and 8.0. In certain embodiments, the buffer system is a phosphate buffer or a histidine buffer. In certain embodiments, the concentration of the phosphate or histidine buffer is between 5 mM and 30 mM. In certain embodiments, the concentration of the phosphate buffer is about 10 to about 20 mM, about 10 to about 15 mM, about 20 to about 30 mM, about 20 to about 25 mM, or about 15 to about 20 mM. In certain embodiments, the salt is a chloride salt. In certain embodiments, the concentration of the chloride salt is between 80 mM and 150 mM. In certain embodiments, the concentration of the salt is about 100 mM, about 110 mM, about 130 mM, or about 150 mM. In certain embodiments, the surfactant is a poloxamer or a polysorbate. In certain embodiments, the concentration of the poloxamer or polysorbate is between 0.01% (w/v) and 0.1% (w/v). In certain embodiments, the carbohydrate is sucrose. In certain embodiments, the concentration of the carbohydrate is between 0.5% (w/v) and 5% (w/v). In certain embodiments, the chloride salt is NaCl. In certain embodiments, the poloxamer is selected from the group consisting of poloxamer 101 (P101), poloxamer 105 (P105), poloxamer 108 (P108), poloxamer 122 (P122), poloxamer 123 (P123), poloxamer 124 (P124), poloxamer 181 (P181), poloxamer 182 (P182), poloxamer 183 (P183), poloxamer 184 (P184), poloxamer 185 (P185), poloxamer 188 (P188), poloxamer 212 (P212), poloxamer 215 (P215), poloxamer 217 (P217), poloxamer 231 (P231), poloxamer 234 (P234), poloxamer 235 (P235), poloxamer 237 (P237), poloxamer 238 (P238), poloxamer 282 (P282), poloxamer 284 (P284), poloxamer 288 (P288), poloxamer 331 (P331), poloxamer 333 (P333), poloxamer 334 (P334), poloxamer 335 (P335), poloxamer 338 (P338), poloxamer 401 (P401), poloxamer 402 (P402), poloxamer 403 (P403), poloxamer 407 (P407), and a combination thereof. In certain embodiments, the polysorbate is selected from the group consisting of polysorbate 20, polysorbate 40, polysorbate 60, polysorbate 80, and a combination thereof. In certain embodiments, the pH of the phosphate or histidine buffer is 6.1, 6.3, 6.5, 6.7, 6.9, 7.1, 7.3, 7.5, 7.7, or 7.9. In certain embodiments, the concentration of the phosphate or histidine buffer is 10 mM, 15 mM, 20 mM, or 25 mM. In certain embodiments, the chloride salt is 100 mM, 110 mM, 130 mM, or 150 mM. In certain embodiments, the concentration of the poloxamer or polysorbate is 0.03% (w/v), 0.05% (w/v), 0.07% (w/v), or 0.09% (w/v). In certain embodiments, the concentration of the carbohydrate is 1% (w/v), 2% (w/v), 3% (w/v), or 4% (w/v). In certain embodiments, the poloxamer is poloxamer 188 (P188). In certain embodiments, the poloxamer is poloxamer 407 (P407).

In one aspect, the present invention is directed to a recombinant lentiviral vector preparation, wherein the recombinant lentiviral vector is isolated from transfected host cells, including CHO cells, HEK293 cells, BHK21 cells, PER.C6 cells, NSO cells, and CAP cells, and wherein the recombinant lentiviral vector preparation comprises: (a) an effective dose of a recombinant lentiviral vector; (b) a TRIS-free buffer system; (c) a salt; (d) a surfactant; and (e) a carbohydrate, wherein the pharmaceutical composition is suitable for systemic administration to a human patient. In certain embodiments, the host cells are CD47-positive host cells. In certain embodiments, the vector further comprises an enhanced transthyretin (ET) promoter. In certain embodiments, the vector further comprises a nucleotide sequence at least 90% identical to the target sequence for miR-142 set forth in SEQ ID NO: 7. In certain embodiments, the vector further comprises a nucleotide sequence at least 80% identical to the Factor VIII (FVIII) coding sequence set forth in SEQ ID NO: 1 or SEQ ID NO: 2 or the Factor IX (FIX) coding sequence set forth in SEQ ID NO: 3. In certain embodiments, the vector comprises the Factor VIII (FVIII) coding sequence set forth in SEQ ID NO: 1 or SEQ ID NO: 2. In certain embodiments, the vector comprises the Factor IX (FIX) coding sequence set forth in SEQ ID NO: 3.

In certain embodiments, the vector further comprises a nucleotide sequence encoding VSV-G or a fragment thereof. In certain embodiments, the pH of the buffer system is between 6.0 and 8.0. In certain embodiments, the buffer system is a phosphate buffer or a histidine buffer. In certain embodiments, the concentration of the phosphate or histidine buffer is between 5 mM and 30 mM. In certain embodiments, the concentration of the phosphate buffer is about 10 to about 20 mM, about 10 to about 15 mM, about 20 to about 30 mM, about 20 to about 25 mM, or about 15 to about 20 mM. In certain embodiments, the salt is a chloride salt. In certain embodiments, the concentration of the chloride salt is between 80 mM and 150 mM. In certain embodiments, the concentration of the salt is about 100 mM, about 110 mM, about 130 mM, or about 150 mM. In certain embodiments, the surfactant is a poloxamer or a polysorbate. In certain embodiments, the concentration of the poloxamer or polysorbate is between 0.01% (w/v) and 0.1% (w/v). In certain embodiments, the carbohydrate is sucrose. In certain embodiments, the concentration of the carbohydrate is between 0.5% (w/v) and 5% (w/v). In certain embodiments, the chloride salt is NaCl. In certain embodiments, the poloxamer is selected from the group consisting of poloxamer 101 (P101), poloxamer 105 (P105), poloxamer 108 (P108), poloxamer 122 (P122), poloxamer 123 (P123), poloxamer 124 (P124), poloxamer 181 (P181), poloxamer 182 (P182), poloxamer 183 (P183), poloxamer 184 (P184), poloxamer 185 (P185), poloxamer 188 (P188), poloxamer 212 (P212), poloxamer 215 (P215), poloxamer 217 (P217), poloxamer 231 (P231), poloxamer 234 (P234), poloxamer 235 (P235), poloxamer 237 (P237), poloxamer 238 (P238), poloxamer 282 (P282), poloxamer 284 (P284), poloxamer 288 (P288), poloxamer 331 (P331), poloxamer 333 (P333), poloxamer 334 (P334), poloxamer 335 (P335), poloxamer 338 (P338), poloxamer 401 (P401), poloxamer 402 (P402), poloxamer 403 (P403), poloxamer 407 (P407), and a combination thereof. In certain embodiments, the polysorbate is selected from the group consisting of polysorbate 20, polysorbate 40, polysorbate 60, polysorbate 80, and a combination thereof. In certain embodiments, the pH of the phosphate or histidine buffer is 6.1, 6.3, 6.5, 6.7, 6.9, 7.1, 7.3, 7.5, 7.7, or 7.9. In certain embodiments, the concentration of the phosphate or histidine buffer is 10 mM, 15 mM, 20 mM, or 25 mM. In certain embodiments, the chloride salt is 100 mM, 110 mM, 130 mM, or 150 mM. In certain embodiments, the concentration of the poloxamer or polysorbate is 0.03% (w/v), 0.05% (w/v), 0.07% (w/v), or 0.09% (w/v). In certain embodiments, the concentration of the carbohydrate is 1% (w/v), 2% (w/v), 3% (w/v), or 4% (w/v). In certain embodiments, the poloxamer is poloxamer 188 (P188). In certain embodiments, the poloxamer is poloxamer 407 (P407).

In one aspect, the present invention is directed to a method of treating a human patient with a disorder, wherein the human patient is systemically administered a recombinant lentiviral vector preparation comprising: (a) (a) an effective dose of a recombinant lentiviral vector; (b) a TRIS-free buffer system; (c) a salt; (d) a surfactant; and (e) a carbohydrate, wherein the pharmaceutical composition is suitable for systemic administration to a human patient. In certain embodiments, the preparation is administered systemically to the human patient. In certain embodiments, the preparation is administered intravenously.

In certain embodiments, the disorder is a bleeding disorder. In certain embodiments, the bleeding disorder is hemophilia A or hemophilia B.

In certain embodiments, the vector further comprises an enhanced transthyretin (ET) promoter. In certain embodiments, the vector further comprises a nucleotide sequence at least 90% identical to the target sequence for miR-142 set forth in SEQ ID NO: 7. In certain embodiments, the vector further comprises a nucleotide sequence at least 80% identical to the Factor VIII (FVIII) coding sequence set forth in SEQ ID NO: 1 or SEQ ID NO: 2 or the Factor IX (FIX) coding sequence set forth in SEQ ID NO: 3. In certain embodiments, the vector comprises the Factor VIII (FVIII) coding sequence set forth in SEQ ID NO: 1 or SEQ ID NO: 2. In certain embodiments, the vector comprises the Factor IX (FIX) coding sequence set forth in SEQ ID NO: 3.

In certain embodiments, the vector further comprises a nucleotide sequence encoding VSV-G or a fragment thereof. In certain embodiments, the pH of the buffer system is between 6.0 and 8.0. In certain embodiments, the buffer system is a phosphate buffer or a histidine buffer. In certain embodiments, the concentration of the phosphate or histidine buffer is between 5 mM and 30 mM. In certain embodiments, the concentration of the phosphate buffer is about 10 to about 20 mM, about 10 to about 15 mM, about 20 to about 30 mM, about 20 to about 25 mM, or about 15 to about 20 mM. In certain embodiments, the salt is a chloride salt. In certain embodiments, the concentration of the chloride salt is between 80 mM and 150 mM. In certain embodiments, the concentration of the salt is about 100 mM, about 110 mM, about 130 mM, or about 150 mM. In certain embodiments, the surfactant is a poloxamer or a polysorbate. In certain embodiments, the concentration of the poloxamer or polysorbate is between 0.01% (w/v) and 0.1% (w/v). In certain embodiments, the carbohydrate is sucrose. In certain embodiments, the concentration of the carbohydrate is between 0.5% (w/v) and 5% (w/v). In certain embodiments, the chloride salt is NaCl. In certain embodiments, the poloxamer is selected from the group consisting of poloxamer 101 (P101), poloxamer 105 (P105), poloxamer 108 (P108), poloxamer 122 (P122), poloxamer 123 (P123), poloxamer 124 (P124), poloxamer 181 (P181), poloxamer 182 (P182), poloxamer 183 (P183), poloxamer 184 (P184), poloxamer 185 (P185), poloxamer 188 (P188), poloxamer 212 (P212), poloxamer 215 (P215), poloxamer 217 (P217), poloxamer 231 (P231), poloxamer 234 (P234), poloxamer 235 (P235), poloxamer 237 (P237), poloxamer 238 (P238), poloxamer 282 (P282), poloxamer 284 (P284), poloxamer 288 (P288), poloxamer 331 (P331), poloxamer 333 (P333), poloxamer 334 (P334), poloxamer 335 (P335), poloxamer 338 (P338), poloxamer 401 (P401), poloxamer 402 (P402), poloxamer 403 (P403), poloxamer 407 (P407), and a combination thereof. In certain embodiments, the polysorbate is selected from the group consisting of polysorbate 20, polysorbate 40, polysorbate 60, polysorbate 80, and a combination thereof. In certain embodiments, the pH of the phosphate or histidine buffer is 6.1, 6.3, 6.5, 6.7, 6.9, 7.1, 7.3, 7.5, 7.7, or 7.9. In certain embodiments, the concentration of the phosphate or histidine buffer is 10 mM, 15 mM, 20 mM, or 25 mM. In certain embodiments, the chloride salt is 100 mM, 110 mM, 130 mM, or 150 mM. In certain embodiments, the concentration of the poloxamer or polysorbate is 0.03% (w/v), 0.05% (w/v), 0.07% (w/v), or 0.09% (w/v). In certain embodiments, the concentration of the carbohydrate is 1% (w/v), 2% (w/v), 3% (w/v), or 4% (w/v). In certain embodiments, the poloxamer is poloxamer 188 (P188). In certain embodiments, the poloxamer is poloxamer 407 (P407).

### B.1. Bleeding Disorders

Bleeding disorders are a result of the impairment of the blood's ability to form a clot at the site of blood vessel injury. There are several types of bleeding disorders, including hemophilia A, hemophilia B, von Willebrand disease, and rare factor deficiencies. Hemophilia A results from a deficiency in Factor VIII (FVIII) caused by a mutated or under-expressed gene for Factor VIII, while hemophilia B results from a deficiency in Factor IX (FIX) caused by a mutated or under-expressed gene for Factor IX.

According to the US Centers for Disease Control and Prevention, hemophilia occurs in approximately 1 in 5,000 live births. There are about 20,000 people with hemophilia in the US. All races and ethnic groups are affected. Hemophilia A is four times as common as hemophilia B while more than half of patients with hemophilia A have the severe form of hemophilia. People suffering from hemophilia require extensive medical monitoring throughout their lives. In the absence of intervention, afflicted individuals suffer from spontaneous bleeding in the joints, which produces severe pain and debilitating immobility. Bleeding into muscles results in the accumulation of blood in those tissues, while spontaneous bleeding in the throat and neck can cause asphyxiation if not immediately treated. Renal bleeding and severe bleeding following surgery, minor accidental injuries, or dental extractions also are prevalent.

Disclosed herein are formulations used to treat a bleeding disease or condition in a subject in need thereof. The bleeding disease or condition is selected from the group consisting of a bleeding coagulation disorder, hemarthrosis, muscle bleed, oral bleed, hemorrhage, hemorrhage into muscles, oral hemorrhage, trauma, trauma capitis, gastrointestinal bleeding, intracranial hemorrhage, intra-abdominal hemorrhage, intrathoracic hemorrhage, bone fracture, central nervous system bleeding, bleeding in the retropharyngeal space, bleeding in the retroperitoneal space, bleeding in the illiopsoas sheath and any combinations thereof. In still other embodiments, the subject is scheduled to undergo a surgery. In yet other embodiments, the treatment is prophylactic or on-demand.

Gene therapy using stable and potent formulations of lentiviral vectors (LVs) show great promise for treating individuals suffering from hemophilia A or B through the stable integration into cells of Factor VIII or Factor IX genes that result in the expression of adequate levels of functional Favor VIII or Factor IX.

Somatic gene therapy has been explored as a possible treatment for bleeding disorders. Gene therapy is a particularly appealing treatment for hemophilia because of its potential to cure the disease through continuous endogenous production of FVIII or FIX following a single administration of a vector encoding the respective clotting factor. Hemophilia A (deficiency in FVIII) and hemophilia B (deficiency in FIX) are well suited for a gene replacement approach because its clinical manifestations are entirely attributable to the lack of a single gene product (FVIII or FIX) that circulates in minute amounts (200 ng/ml) in the plasma.

Lentiviruses are gaining prominence as gene delivery vehicles due to their large capacity and ability to sustain transgene expression via integration. Lentiviruses have been evaluated in numerous ex-vivo cell therapy clinical programs with promising efficacy and safety profiles, gaining wide experience over the past ten years. As the use of lentiviral in vivo gene therapy is gaining popularity, there is a need in the art for providing improved formulations that enhance the stability of lentiviruses for long term storage.

The present disclosure meets an important need in the art by providing formulation buffers, or vehicles, that confer lentivirus stability, which affords long term frozen storage. In certain exemplary embodiments, the formulation buffers confer lentivirus stability and affords long term frozen storage where the route of administration is systemic. In some embodiments, the lentivirus is processed into a formulation buffer, or vehicle, of the present disclosure after purification. Upon formulating, the lentivirus is stored frozen. A formulation buffer, or vehicle, of the present invention offers enhanced stability upon freezing and thawing as well as exposure to elevated temperatures.

Provided herein are lentiviral vectors comprising a codon optimized FVIII sequence or codon optimized FIX sequence that demonstrates increased expression in a subject and potentially results in greater therapeutic efficacy when used in gene therapy methods. Embodiments of the present disclosure are directed to lentiviral vectors comprising one or more codon optimized nucleic acid molecules encoding a polypeptide with FVIII activity, or lentiviral vectors comprising one or more codon optimized nucleic acid molecules encoding a polypeptide with FIX activity as described herein, host cells (e.g., hepatocytes) comprising the lentiviral vectors, and methods of use of the disclosed lentiviral vectors (e.g., treatments for bleeding disorders using the lentiviral vectors disclosed herein). In certain embodiments, during scale-up processing, the lentiviral vector is packaged into lentivirus that is processed into a formulation buffer, or vehicle, of the present disclosure.

In general, the methods of treatment disclosed herein involve administration of a lentiviral vector comprising a nucleic acid molecule comprising at least one codon optimized nucleic acid sequence encoding a FVIII clotting factor, or a lentiviral vector comprising a nucleic acid molecule comprising at least one codon optimized nucleic acid sequence encoding a FIX clotting factor. In some embodiments, the nucleic acid sequence encoding a FVIII clotting factor is operably linked to suitable expression control sequences, which in some embodiments are incorporated into the lentiviral vector (e.g., a replication-defective lentiviral viral vector). In some embodiments, the nucleic acid sequence encoding a FIX clotting factor is operably linked to suitable expression control sequences, which in some embodiments are incorporated into the lentiviral vector (e.g., a replication-defective lentiviral viral vector).

The present disclosure provides methods of treating a bleeding disorder (e.g., hemophilia A or hemophilia B) in a subject in need thereof comprising administering to the subject at least one dose of a lentiviral vector comprising a nucleic acid molecule comprising a nucleotide sequence encoding a polypeptide with FVIII or FIX activity. In certain embodiments, the lentiviral vector is packaged into lentivirus that is processed into a formulation buffer of the present invention. In certain embodiments, the nucleotide sequence encoding a polypeptide with FVIII activity comprises a nucleotide sequence having at least 80%, at least 81%, at least 82%, at least 83%, at least 84%, at least 85%, at least 86%, at least 87%, at least 88%, or at least 89%, at least 90%, at least 91%, at least 92%, at least 93%, at least 94%, at least 95%, at least 96%, at least 97%, at least 98%, at least 99%, or at least 100% sequence identity to the nucleic acid sequence set forth in SEQ ID NO:1, as shown in Table 1. In certain embodiments the nucleotide sequence encoding a polypeptide with FVIII activity consists of the nucleotide sequence set forth in SEQ ID NO:1, as shown in Table 1. In certain embodiments, the nucleotide sequence encoding a polypeptide with FVIII activity comprises a nucleotide sequence having at least 80%, at least 81%, at least 82%, at least 83%, at least 84%, at least 85%, at least 86%, at least 87%, at least 88%, or at least 89%, at least 90%, at least 91%, at least 92%, at least 93%, at least 94%, at least 95%, at least 96%, at least 97%, at least 98%, at least 99%, or at least 100% sequence identity to the nucleic acid sequence set forth in SEQ ID NO:2, as shown in Table 1. In certain embodiments, the nucleotide sequence encoding a polypeptide with FVIII activity consists of the nucleotide sequence set forth in SEQ ID NO:2, as shown in Table 1. In certain embodiments, the nucleotide sequence encoding a polypeptide with FIX activity comprises a nucleotide sequence having at least 80%, at least 81%, at least 82%, at least 83%, at least 84%, at least 85%, at least 86%, at least 87%, at least 88%, or at least 89%, at least 90%, at least 91%, at least 92%, at least 93%, at least 94%, at least 95%, at least 96%, at least 97%, at least 98%, at least 99%, or at least 100% sequence identity to the nucleic acid sequence set forth in SEQ ID NO:3, as shown in Table 1. In certain embodiments, the nucleotide sequence encoding a polypeptide with FIX activity consists of the nucleotide sequence set forth in SEQ ID NO:3, as shown in Table 1.

The present disclosure provides methods of treating a bleeding disorder (e.g., hemophilia A or hemophilia B) in a subject in need thereof comprising administering to the subject at least one dose a lentiviral vector comprising nucleic acid molecule comprising a nucleotide sequence encoding a polypeptide with FVIII or FIX activity. In certain embodiments, the lentiviral vector is packaged into lentivirus that is processed into a formulation buffer, or vehicle, of the present invention. In certain embodiments, the subject is administered at least one dose of 5×10¹⁰ or less transducing units/kg (TU/kg) (or 10⁹ TU/kg or less, or 108 TU/kg or less) of a lentiviral vector comprising nucleic acid molecule comprising a nucleotide sequence encoding a polypeptide with FVIII or FIX activity as described herein.

In some embodiments, the dose is about 5.0×10¹⁰ TU/kg, about 4.9×10¹⁰ TU/kg, about 4.8×10¹⁰ TU/kg, about 4.7×10¹⁰ TU/kg, about 4.6×10¹⁰ TU/kg, about 4.5×10¹⁰ TU/kg, about 4.4×10¹⁰ TU/kg, about 4.3×10¹⁰ TU/kg, about 4.2×10¹⁰ TU/kg, about 4.1×10¹⁰ TU/kg, about 4.0×10¹⁰ TU/kg, about 3.9×10¹⁰ TU/kg, about 3.8×10¹⁰ TU/kg, about 3.7×10¹⁰ TU/kg, about 3.6×10¹⁰ TU/kg, about 3.5×10¹⁰ TU/kg, about 3.4×10¹⁰ TU/kg, about 3.3×10¹⁰ TU/kg, about 3.2×10¹⁰ TU/kg, about 3.1×10¹⁰ TU/kg, about 3.0×10¹⁰ TU/kg, about 2.9×10¹⁰ TU/kg, about 2.8×10¹⁰ TU/kg, about 2.7×10¹⁰ TU/kg, about 2.6×10¹⁰ TU/kg, about 2.5×10¹⁰ TU/kg, about 2.4×10¹⁰ TU/kg, about 2.3×10¹⁰ TU/kg, about 2.2×10¹⁰ TU/kg, about 2.1×10¹⁰ TU/kg, about 2.0×10¹⁰ TU/kg, about 1.9×10¹⁰ TU/kg, about 1.8×10¹⁰ TU/kg, about 1.7×10¹⁰ TU/kg, about 1.6×10¹⁰ TU/kg, about 1.5×10¹⁰ TU/kg, about 1.4×10¹⁰ TU/kg, about 1.3×10¹⁰ TU/kg, about 1.2×10¹⁰ TU/kg, about 1.1×10¹⁰ TU/kg, or about 1.0×10¹⁰ TU/kg.

In some embodiments, the dose is about 9.9×10⁹ TU/kg, about 9.8×10⁹ TU/kg, about 9.7×10⁹ TU/kg, about 9.6×10⁹ TU/kg, about 9.5×10⁹ TU/kg, about 9.4×10⁹ TU/kg, about 9.3×10⁹ TU/kg, about 9.2×10⁹ TU/kg, about 9.1×10⁹ TU/kg, about 9.0×10⁹ TU/kg, about 8.9×10⁹ TU/kg, about 8.8×10⁹ TU/kg, about 8.7×10⁹ TU/kg, about 8.6×10⁹ TU/kg, about 8.5×10⁹ TU/kg, about 8.4×10⁹ TU/kg, about 8.3×10⁹ TU/kg, about 8.2×10⁹ TU/kg, about 8.1×10⁹ TU/kg, about 8.0×10⁹ TU/kg, about 7.9×10⁹ TU/kg, about 7.8×10⁹ TU/kg, about 7.7×10⁹ TU/kg, about 7.6×10⁹ TU/kg, about 7.5×10⁹ TU/kg, about 7.4×10⁹ TU/kg, about 7.3×10⁹ TU/kg, about 7.2×10⁹ TU/kg, about 7.1×10⁹ TU/kg, about 7.0×10⁹ TU/kg, about 6.9×10⁹ TU/kg, about 6.8×10⁹ TU/kg, about 6.7×10⁹ TU/kg, about 6.6×10⁹ TU/kg, about 6.5×10⁹ TU/kg, about 6.4×10⁹ TU/kg, about 6.3×10⁹ TU/kg, about 6.2×10⁹ TU/kg, about 6.1×10⁹ TU/kg, about 6.0×10⁹ TU/kg, about 5.9×10⁹ TU/kg, about 5.8×10⁹ TU/kg, about 5.7×10⁹ TU/kg, about 5.6×10⁹ TU/kg, about 5.5×10⁹ TU/kg, about 5.4×10⁹ TU/kg, about 5.3×10⁹ TU/kg, about 5.2×10⁹ TU/kg, about 5.1×10⁹ TU/kg, about 5.0×10⁹ TU/kg, about 4.9×10⁹ TU/kg, about 4.8×10⁹ TU/kg, about 4.7×10⁹ TU/kg, about 4.6×10⁹ TU/kg, about 4.5×10⁹ TU/kg, about 4.4×10⁹ TU/kg, about 4.3×10⁹ TU/kg, about 4.2×10⁹ TU/kg, about 4.1×10⁹ TU/kg, about 4.0×10⁹ TU/kg, about 3.9×10⁹ TU/kg, about 3.8×10⁹ TU/kg, about 3.7×10⁹ TU/kg, about 3.6×10⁹ TU/kg, about 3.5×10⁹ TU/kg, about 3.4×10⁹ TU/kg, about 3.3×10⁹ TU/kg, about 3.2×10⁹ TU/kg, about 3.1×10⁹ TU/kg, about 3.0×10⁹ TU/kg, about 2.9×10⁹ TU/kg, about 2.8×10⁹ TU/kg, about 2.7×10⁹ TU/kg, about 2.6×10⁹ TU/kg, about 2.5×10⁹ TU/kg, about 2.4×10⁹ TU/kg, about 2.3×10⁹ TU/kg, about 2.2×10⁹ TU/kg, about 2.1×10⁹ TU/kg, about 2.0×10⁹ TU/kg, about 1.9×10⁹ TU/kg, about 1.8×10⁹ TU/kg, about 1.7×10⁹ TU/kg, about 1.6×10⁹ TU/kg, about 1.5×10⁹ TU/kg, about 1.4×10⁹ TU/kg. about 1.3×10⁹ TU/kg, about 1.2×10⁹ TU/kg, about 1.1×10⁹ TU/kg, or about 1.0×10⁹ TU/kg.

In some embodiments, the dose is about 9.9×10⁸ TU/kg, about 9.8×10⁸ TU/kg, about 9.7×10⁸ TU/kg, about 9.6×10⁸ TU/kg, about 9.5×10⁸ TU/kg, about 9.4×10⁸ TU/kg, about 9.3×10⁸ TU/kg, about 9.2×10⁸ TU/kg, about 9.1×10⁸ TU/kg, about 9.0×10⁸ TU/kg, about 8.9×10⁸ TU/kg, about 8.8×10⁸ TU/kg, about 8.7×10⁸ TU/kg, about 8.6×10⁸ TU/kg, about 8.5×10⁸ TU/kg, about 8.4×10⁸ TU/kg, about 8.3×10⁸ TU/kg, about 8.2×10⁸ TU/kg, about 8.1×10⁸ TU/kg, about 8.0×10⁸ TU/kg, about 7.9×10⁸ TU/kg, about 7.8×10⁸ TU/kg, about 7.7×10⁸ TU/kg, about 7.6×10⁸ TU/kg, about 7.5×10⁸ TU/kg, about 7.4×10⁸ TU/kg, about 7.3×10⁸ TU/kg, about 7.2×10⁸ TU/kg, about 7.1×10⁸ TU/kg, about 7.0×10⁸ TU/kg, about 6.9×10⁸ TU/kg, about 6.8×10⁸ TU/kg, about 6.7×10⁸ TU/kg, about 6.6×10⁸ TU/kg, about 6.5×10⁸ TU/kg, about 6.4×10⁸ TU/kg, about 6.3×10⁸ TU/kg, about 6.2×10⁸ TU/kg, about 6.1×10⁸ TU/kg, about 6.0×10⁸ TU/kg, about 5.9×10⁸ TU/kg, about 5.8×10⁸ TU/kg, about 5.7×10⁸ TU/kg, about 5.6×10⁸ TU/kg, about 5.5×10⁸ TU/kg, about 5.4×10⁸ TU/kg, about 5.3×10⁸ TU/kg, about 5.2×10⁸ TU/kg, about 5.1×10⁸ TU/kg, about 5.0×10⁸ TU/kg, about 4.9×10⁸ TU/kg, about 4.8×10⁸ TU/kg, about 4.7×10⁸ TU/kg, about 4.6×10⁸ TU/kg, about 4.5×10⁸ TU/kg, about 4.4×10⁸ TU/kg, about 4.3×10⁸ TU/kg, about 4.2×10⁸ TU/kg, about 4.1×10⁸ TU/kg, about 4.0×10⁸ TU/kg, about 3.9×10⁸ TU/kg, about 3.8×10⁸ TU/kg, about 3.7×10⁸ TU/kg, about 3.6×10⁸ TU/kg, about 3.5×10⁸ TU/kg, about 3.4×10⁸ TU/kg, about 3.3×10⁸ TU/kg, about 3.2×10⁸ TU/kg, about 3.1×10⁸ TU/kg, about 3.0×10⁸ TU/kg, about 2.9×10⁸ TU/kg, about 2.8×10⁸ TU/kg, about 2.7×10⁸ TU/kg, about 2.6×10⁸ TU/kg, about 2.5×10⁸ TU/kg, about 2.4×10⁸ TU/kg, about 2.3×10⁸ TU/kg, about 2.2×10⁸ TU/kg, about 2.1×10⁸ TU/kg, about 2.0×10⁸ TU/kg, about 1.9×10⁸ TU/kg, about 1.8×10⁸ TU/kg, about 1.7×10⁸ TU/kg, about 1.6×10⁸ TU/kg, about 1.5×10⁸ TU/kg, about 1.4×10⁸ TU/kg. about 1.3×10⁸ TU/kg, about 1.2×10⁸ TU/kg, about 1.1×10⁸ TU/kg, or about 1.0×10⁸ TU/kg.

In some embodiments, the dose is less than 5.0×10¹⁰ TU/kg, less than 4.9×10¹⁰ TU/kg, less than 4.8×10¹⁰ TU/kg, less than 4.7×10¹⁰ TU/kg, less than 4.6×10¹⁰ TU/kg, less than 4.5×10¹⁰ TU/kg, less than 4.4×10¹⁰ TU/kg, less than 4.3×10¹⁰ TU/kg, less than 4.2×10¹⁰ TU/kg, less than 4.1×10¹⁰ TU/kg, less than 4.0×10¹⁰ TU/kg, less than 3.9×10¹⁰ TU/kg, less than 3.8×10¹⁰ TU/kg, less than 3.7×10¹⁰ TU/kg, less than 3.6×10¹⁰ TU/kg, less than 3.5×10¹⁰ TU/kg, less than 3.4×10¹⁰ TU/kg, less than 3.3×10¹⁰ TU/kg, less than 3.2×10¹⁰ TU/kg, less than 3.1×10¹⁰ TU/kg, less than 3.0×10¹⁰ TU/kg, less than 2.9×10¹⁰ TU/kg, less than 2.8×10¹⁰ TU/kg, less than 2.7×10¹⁰ TU/kg, less than 2.6×10¹⁰ TU/kg, less than 2.5×10¹⁰ TU/kg, less than 2.4×10¹⁰ TU/kg, less than 2.3×10¹⁰ TU/kg, less than 2.2×10¹⁰ TU/kg, less than 2.1×10¹⁰ TU/kg, less than 2.0×10¹⁰ TU/kg, less than 1.9×10¹⁰ TU/kg, less than 1.8×10¹⁰ TU/kg, less than 1.7×10¹⁰ TU/kg, less than 1.6×10¹⁰ TU/kg, less than 1.5×10¹⁰ TU/kg, less than 1.4×10¹⁰ TU/kg, less than 1.3×10¹⁰ TU/kg, less than 1.2×10¹⁰ TU/kg, less than 1.1×10¹⁰ TU/kg, or less than 1.0×10¹⁰ TU/kg.

In some embodiments, the dose is less than 9.9×10⁹ TU/kg, less than 9.8×10⁹ TU/kg, less than 9.7×10⁹ TU/kg, less than 9.6×10⁹ TU/kg, less than 9.5×10⁹ TU/kg, less than 9.4×10⁹ TU/kg, less than 9.3×10⁹ TU/kg, less than 9.2×10⁹ TU/kg, less than 9.1×10⁹ TU/kg, less than 9.0×10⁹ TU/kg, less than 8.9×10⁹ TU/kg, less than 8.8×10⁹ TU/kg, less than 8.7×10⁹ TU/kg, less than 8.6×10⁹ TU/kg, less than 8.5×10⁹ TU/kg, less than 8.4×10⁹ TU/kg, less than 8.3×10⁹ TU/kg, less than 8.2×10⁹ TU/kg, less than 8.1×10⁹ TU/kg, less than 8.0×10⁹ TU/kg, less than 7.9×10⁹ TU/kg, less than 7.8×10⁹ TU/kg, less than 7.7×10⁹ TU/kg, less than 7.6×10⁹ TU/kg, less than 7.5×10⁹ TU/kg, less than 7.4×10⁹ TU/kg, less than 7.3×10⁹ TU/kg, less than 7.2×10⁹ TU/kg, less than 7.1×10⁹ TU/kg, less than 7.0×10⁹ TU/kg, less than 6.9×10⁹ TU/kg, less than 6.8×10⁹ TU/kg, less than 6.7×10⁹ TU/kg, less than 6.6×10⁹ TU/kg, less than 6.5×10⁹ TU/kg, less than 6.4×10⁹ TU/kg, less than 6.3×10⁹ TU/kg, less than 6.2×10⁹ TU/kg, less than 6.1×10⁹ TU/kg, less than 6.0×10⁹ TU/kg, less than 5.9×10⁹ TU/kg, less than 5.8×10⁹ TU/kg, less than 5.7×10⁹ TU/kg, less than 5.6×10⁹ TU/kg, less than 5.5×10⁹ TU/kg, less than 5.4×10⁹ TU/kg, less than 5.3×10⁹ TU/kg, less than 5.2×10⁹ TU/kg, less than 5.1×10⁹ TU/kg, less than 5.0×10⁹ TU/kg, less than 4.9×10⁹ TU/kg, less than 4.8×10⁹ TU/kg, less than 4.7×10⁹ TU/kg, less than 4.6×10⁹ TU/kg, less than 4.5×10⁹ TU/kg, less than 4.4×10⁹ TU/kg, less than 4.3×10⁹ TU/kg, less than 4.2×10⁹ TU/kg, less than 4.1×10⁹ TU/kg, less than 4.0×10⁹ TU/kg, less than 3.9×10⁹ TU/kg, less than 3.8×10⁹ TU/kg, less than 3.7×10⁹ TU/kg, less than 3.6×10⁹ TU/kg, less than 3.5×10⁹ TU/kg, less than 3.4×10⁹ TU/kg, less than 3.3×10⁹ TU/kg, less than 3.2×10⁹ TU/kg, less than 3.1×10⁹ TU/kg, less than 3.0×10⁹ TU/kg, less than 2.9×10⁹ TU/kg, less than 2.8×10⁹ TU/kg, less than 2.7×10⁹ TU/kg, less than 2.6×10⁹ TU/kg, less than 2.5×10⁹ TU/kg, less than 2.4×10⁹ TU/kg, less than 2.3×10⁹ TU/kg, less than 2.2×10⁹ TU/kg, less than 2.1×10⁹ TU/kg, less than 2.0×10⁹ TU/kg, less than 1.9×10⁹ TU/kg, less than 1.8×10⁹ TU/kg, less than 1.7×10⁹ TU/kg, less than 1.6×10⁹ TU/kg, less than 1.5×10⁹ TU/kg, less than 1.4×10⁹ TU/kg, less than 1.3×10⁹ TU/kg, less than 1.2×10⁹ TU/kg, less than 1.1×10⁹ TU/kg, or less than 1.0×10⁹ TU/kg.

In some embodiments, the dose is less than 9.9×10⁸ TU/kg, less than 9.8×10⁸ TU/kg, less than 9.7×10⁸ TU/kg, less than 9.6×10⁸ TU/kg, less than 9.5×10⁸ TU/kg, less than 9.4×10⁸ TU/kg, less than 9.3×10⁸ TU/kg, less than 9.2×10⁸ TU/kg, less than 9.1×10⁸ TU/kg, less than 9.0×10⁸ TU/kg, less than 8.9×10⁸ TU/kg, less than 8.8×10⁸ TU/kg, less than 8.7×10⁸ TU/kg, less than 8.6×10⁸ TU/kg, less than 8.5×10⁸ TU/kg, less than 8.4×10⁸ TU/kg, less than 8.3×10⁸ TU/kg, less than 8.2×10⁸ TU/kg, less than 8.1×10⁸ TU/kg, less than 8.0×10⁸ TU/kg, less than 7.9×10⁸ TU/kg, less than 7.8×10⁸ TU/kg, less than 7.7×10⁸ TU/kg, less than 7.6×10⁸ TU/kg, less than 7.5×10⁸ TU/kg, less than 7.4×10⁸ TU/kg, less than 7.3×10⁸ TU/kg, less than 7.2×10⁸ TU/kg, less than 7.1×10⁸ TU/kg, less than 7.0×10⁸ TU/kg, less than 6.9×10⁸ TU/kg, less than 6.8×10⁸ TU/kg, less than 6.7×10⁸ TU/kg, less than 6.6×10⁸ TU/kg, less than 6.5×10⁸ TU/kg, less than 6.4×10⁸ TU/kg, less than 6.3×10⁸ TU/kg, less than 6.2×10⁸ TU/kg, less than 6.1×10⁸ TU/kg, less than 6.0×10⁸ TU/kg, less than 5.9×10⁸ TU/kg, less than 5.8×10⁸ TU/kg, less than 5.7×10⁸ TU/kg, less than 5.6×10⁸ TU/kg, less than 5.5×10⁸ TU/kg, less than 5.4×10⁸ TU/kg, less than 5.3×10⁸ TU/kg, less than 5.2×10⁸ TU/kg, less than 5.1×10⁸ TU/kg, less than 5.0×10⁸ TU/kg, less than 4.9×10⁸ TU/kg, less than 4.8×10⁸ TU/kg, less than 4.7×10⁸ TU/kg, less than 4.6×10⁸ TU/kg, less than 4.5×10⁸ TU/kg, less than 4.4×10⁸ TU/kg, less than 4.3×10⁸ TU/kg, less than 4.2×10⁸ TU/kg, less than 4.1×10⁸ TU/kg, less than 4.0×10⁸ TU/kg, less than 3.9×10⁸ TU/kg, less than 3.8×10⁸ TU/kg, less than 3.7×10⁸ TU/kg, less than 3.6×10⁸ TU/kg, less than 3.5×10⁸ TU/kg, less than 3.4×10⁸ TU/kg, less than 3.3×10⁸ TU/kg, less than 3.2×10⁸ TU/kg, less than 3.1×10⁸ TU/kg, less than 3.0×10⁸ TU/kg, less than 2.9×10⁸ TU/kg, less than 2.8×10⁸ TU/kg, less than 2.7×10⁸ TU/kg, less than 2.6×10⁸ TU/kg, less than 2.5×10⁸ TU/kg, less than 2.4×10⁸ TU/kg, less than 2.3×10⁸ TU/kg, less than 2.2×10⁸ TU/kg, less than 2.1×10⁸ TU/kg, less than 2.0×10⁸ TU/kg, less than 1.9×10⁸ TU/kg, less than 1.8×10⁸ TU/kg, less than 1.7×10⁸ TU/kg, less than 1.6×10⁸ TU/kg, less than 1.5×10⁸ TU/kg, less than 1.4×10⁸ TU/kg, less than 1.3×10⁸ TU/kg, less than 1.2×10⁸ TU/kg, less than 1.1×10⁸ TU/kg, or less than 1.0×10⁸ TU/kg.

In some embodiments, the dose is between 1×10⁸ TU/kg and 5×10¹⁰ TU/kg, between 1.5×10⁸ TU/kg and 5×10¹⁰ TU/kg, between 2×10⁸ TU/kg and 5×10¹⁰ TU/kg, between 2.5×10⁸ TU/kg and 5×10¹⁰ TU/kg, between 3×10⁸ TU/kg and 5×10¹⁰ TU/kg, between 3.5×10⁸ TU/kg and 5×10¹⁰ TU/kg, between 4×10⁸ TU/kg and 5×10¹⁰ TU/kg, between 4.5×10⁸ TU/kg and 5×10¹⁰ TU/kg, between 5×10⁸ TU/kg and 5×10¹⁰ TU/kg, between 5.5×10⁸ TU/kg and 5×10¹⁰ TU/kg, between 6×10⁸ TU/kg and 5×10¹⁰ TU/kg, between 6.5×10⁸ TU/kg and 5×10¹⁰ TU/kg, between 7×10⁸ TU/kg and 5×10¹⁰ TU/kg, between 7.5×10⁸ TU/kg and 5×10¹⁰ TU/kg, between 8×10⁸ TU/kg and 5×10¹⁰ TU/kg, between 8.5×10⁸ TU/kg and 5×10¹⁰ TU/kg, between 9×10⁸ TU/kg and 5×10¹⁰ TU/kg, between 9.5×10⁸ TU/kg and 5×10¹⁰ TU/kg, between 1×10⁹ TU/kg and 5×10¹⁰ TU/kg, between 1.5×10⁹ TU/kg and 5×10¹⁰ TU/kg, between 2×10⁹ TU/kg and 5×10¹⁰ TU/kg, between 2.5×10⁹ TU/kg and 5×10¹⁰ TU/kg, between 3×10⁹ TU/kg and 5×10¹⁰ TU/kg, between 3.5×10⁹ TU/kg and 5×10¹⁰ TU/kg, between 4×10⁹ TU/kg and 5×10¹⁰ TU/kg, between 4.5×10⁹ TU/kg and 5×10¹⁰ TU/kg, between 5×10⁹ TU/kg and 5×10¹⁰ TU/kg, between 5.5×10⁹ TU/kg and 5×10¹⁰ TU/kg, between 6×10⁹ TU/kg and 5×10¹⁰ TU/kg, between 6.5×10⁹ TU/kg and 5×10¹⁰ TU/kg, between 7×10⁹ TU/kg and 5×10¹⁰ TU/kg, between 7.5×10⁹ TU/kg and 5×10¹⁰ TU/kg, between 8×10⁹ TU/kg and 5×10¹⁰ TU/kg, between 8.5×10⁹ TU/kg and 5×10¹⁰ TU/kg, between 9×10⁹ TU/kg and 5×10¹⁰ TU/kg, between 9.5×10⁹ TU/kg and 5×10¹⁰ TU/kg, between 10¹⁰ TU/kg and 5×10¹⁰ TU/kg, between 1.5×10¹⁰ TU/kg and 5×10¹⁰ TU/kg, between 2×10¹⁰ TU/kg and 5×10¹⁰ TU/kg, between 2.5×10¹⁰ TU/kg and 5×10¹⁰ TU/kg, between 3×10¹⁰ TU/kg and 5×10¹⁰ TU/kg, between 3.5×10¹⁰ TU/kg and 5×10¹⁰ TU/kg, between 4×10¹⁰ TU/kg and 5×10¹⁰ TU/kg, or between 4.5×10¹⁰ TU/kg and 5×10¹⁰ TU/kg.

In some embodiments, the dose is between 1×10⁸ TU/kg and 5×10¹⁰ TU/kg, between 1×10⁸ TU/kg and 4.5×10¹⁰ TU/kg, between 1×10⁸ TU/kg and 4×10¹⁰ TU/kg, between 1×10⁸ TU/kg and 3.5×10¹⁰ TU/kg, between 1×10⁸ TU/kg and 3×10¹⁰ TU/kg, between 1×10⁸ TU/kg and 2.5×10¹⁰ TU/kg, between 1×10⁸ TU/kg and 2×10¹⁰ TU/kg, between 1×10⁸ TU/kg and 1.5×10¹⁰ TU/kg, between 1×10⁸ TU/kg and 10¹⁰ TU/kg, between 1×10⁸ TU/kg and 9×10⁹ TU/kg, between 1×10⁸ TU/kg and 8.5×10⁹ TU/kg, between 1×10⁸ TU/kg and 8×10⁹ TU/kg, between 1×10⁸ TU/kg and 7.5×10⁹ TU/kg, between 1×10⁸ TU/kg and 7×10⁹ TU/kg, between 1×10⁸ TU/kg and 6.5×10⁹ TU/kg, between 1×10⁸ TU/kg and 6×10⁹ TU/kg, between 1×10⁸ TU/kg and 5.5×10⁹ TU/kg, between 1×10⁸ TU/kg and 5×10⁹ TU/kg, between 1×10⁸ TU/kg and 4.5×10⁹ TU/kg, between 1×10⁸ TU/kg and 4×10⁹ TU/kg, between 1×10⁸ TU/kg and 3.5×10⁹ TU/kg, between 1×10⁸ TU/kg and 3×10⁹ TU/kg, between 1×10⁸ TU/kg and 2.5×10⁹ TU/kg, between 1×10⁸ TU/kg and 2×10⁹, between 1×10⁸ TU/kg and 1.5×10⁹ TU/kg, between 1×10⁸ TU/kg and 1×10⁹ TU/kg, between 1×10⁸ TU/kg and 9.5×10⁸ TU/kg, between 1×10⁸ TU/kg and 9×10⁸ TU/kg, between 1×10⁸ TU/kg and 8.5×10⁸ TU/kg, between 1×10⁸ TU/kg and 8×10⁸ TU/kg, between 1×10⁸ TU/kg and 7.5×10⁸ TU/kg, between 1×10⁸ TU/kg and 7×10⁸ TU/kg, between 1×10⁸ TU/kg and 6.5×10⁸ TU/kg, between 1×10⁸ TU/kg and 6×10⁸ TU/kg, between 1×10⁸ TU/kg and 5.5×10⁸ TU/kg, between 1×10⁸ TU/kg and 5×10⁸ TU/kg, between 1×10⁸ TU/kg and 4.5×10⁸ TU/kg, between 1×10⁸ TU/kg and 4×10⁸ TU/kg, between 1×10⁸ TU/kg and 3.5×10⁸ TU/kg, between 1×10⁸ TU/kg and 3×10⁸ TU/kg, between 1×10⁸ TU/kg and 2.5×10⁸ TU/kg, between 1×10⁸ TU/kg and 2×10⁸, or between 1×10⁸ TU/kg and 1.5×10⁸ TU/kg,

In some embodiments, the dose is between 1×10¹⁰ TU/kg and 2×10¹⁰ TU/kg, between 1.1×10¹⁰ TU/kg and 1.9×10¹⁰ TU/kg, between 1.2×10¹⁰ TU/kg and 1.8×10¹⁰ TU/kg, between 1.3×10¹⁰ TU/kg and 1.7×10¹⁰ TU/kg, or between 1.4×10¹⁰ TU/kg and 1.6×10¹⁰ TU/kg. In some embodiments, the dose is about 1.5×10¹⁰ TU/kg. In some embodiments, the dose is 1.5×10¹⁰ TU/kg.

In some embodiments, the dose is between 1×10⁹ TU/kg and 2×10⁹ TU/kg, between 1.1×10⁹ TU/kg and 1.9×10⁹ TU/kg, between 1.2×10⁹ TU/kg and 1.8×10⁹ TU/kg, between 1.3×10⁹ TU/kg and 1.7×10⁹ TU/kg, or between 1.4×10⁹ TU/kg and 1.6×10⁹ TU/kg. In some embodiments, the dose is 1.5×10⁹ TU/kg. In certain embodiments, the dose is about 3.0×10⁹ TU/kg.

In some embodiments, plasma FVIII activity at 24 hours, 36 hours, or 48 hours post administration of a lentiviral vector of the present disclosure is increased relative to the plasma FVIII activity in a subject administered a control lentiviral vector. In some embodiments, plasma FVIII activity at 24 hours, 36 hours, or 48 hours post administration of a lentiviral vector of the present disclosure is increased relative to the plasma FVIII activity in a subject administered a control nucleic acid molecule.

In some embodiments, plasma FIX activity at 24 hours, 36 hours, or 48 hours post administration of a lentiviral vector of the present disclosure is increased relative to the plasma FIX activity in a subject administered a control lentiviral vector. In some embodiments, plasma FIX activity at 24 hours, 36 hours, or 48 hours post administration of a lentiviral vector of the present disclosure is increased relative to the plasma FIX activity in a subject administered a control nucleic acid molecule.

In some embodiments, plasma FVIII or plasma FIX activity is increased at about 6 hours, at about 12 hours, at about 18 hours, at about 24 hours, at about 36 hours, at about 48 hours, at about 3 days, at about 4 days, at about 5 days, at about 6 days, at about 7 days, at about 8 days, at about 9 days, at about 10 days, at about 11 days, at about 12 days, at about 13 days, at about 14 days, at about 15 days, at about 16 days, at about 17 days, at about 18 days, at about 19 days, at about 20 days, at about 21 days, at about 22 days, at about 23 days, at about 24 days, at about 25 days, at about 26 days, at about 27 days, or at about 28 days post administration of a lentiviral vector of the present disclosure relative to a subject administered a control lentiviral vector or a control nucleic acid molecule.

In some embodiments, the plasma FVIII or plasma FIX activity in the subject is increased by at least about 2-fold, at least about 3-fold, at least about 4-fold, at least about 5-fold, at least about 6-fold, at least about 7-fold, at least about 8-fold, at least about 9-fold, at least about 10-fold, at least about 11-fold, at least about 12-fold, at least about 13-fold, at least about 14-fold, at least about 15-fold, at least about 20-fold, at least about 25-fold, at least about 30-fold, at least about 35-fold, at least about 40-fold, at least about 45-fold, at least about 50-fold, at least about 55-fold, at least about 60-fold, at least about 65-fold, at least about 70-fold, at least about 75-fold, at least about 80-fold, at least about 85-fold, at least about 90-fold, at least about 95-fold, at least about 100-fold, at least about 110-fold, at least about 120-fold, at least about 130-fold, at least about 140-fold, at least about 150-fold, at least about 160-fold, at least about 170-fold, at least about 180-fold, at least about 190-fold, or at least about 200-fold with respect to basal levels in the subject, relative to levels in a subject administered a control lentiviral vector or a control nucleic acid molecule.

In some embodiments, the lentiviral vector is administered as a single dose or multiple doses. In some embodiments, the lentiviral vector dose is administered at once or divided into multiple sub-dose, e.g., two sub-doses, three sub-doses, four sub-doses, five sub-doses, six sub-doses, or more than six sub-doses. In some embodiments, more than one lentiviral vector is administered.

In some embodiments, the dose of lentiviral vector is administered repeated at least twice, at least three times, at least four times, at least five times, at least six times, at least seven times, at least eight times, at least nine times, or at least ten times. In some embodiments, the lentiviral vector is administered via intravenous injection.

In some embodiments, the subject is a pediatric subject. In some embodiments, the subject is an adult subject.

In some embodiments, the lentiviral vector comprises at least one tissue specific promoter, i.e., a promoter that would regulate the expression of the polypeptide with FVIII activity or the polypeptide with FIX activity in a particular tissue or cell type. In some embodiments, a tissue specific promoter in the lentiviral vector selectively enhances expression of the polypeptide with FVIII activity in a target liver cell. In some embodiments, the tissue specific promoter that selectively enhances expression of the polypeptide with FVIII activity in a target liver cell comprises an mTTR promoter. In some embodiments, the tissue specific promoter that selectively enhances expression of the polypeptide with FIX activity in a target liver cell comprises an APOA2 promoter, SERPINA1 (hAAT) promoter, mTTR promoter, MIR122 promoter, the ET promoter (GenBank No. AY661265; see also Vigna et al., Molecular Therapy 11(5):763 (2005)), or any combination thereof. In some embodiments, the target liver cell is a hepatocyte.

Since the lentiviral vector can transduce all liver cell types, the expression of the transgene (e.g., FVIII or FIX) in different cell types can be controlled by using different promoters in the lentiviral vector. Thus, the lentiviral vector can comprise specific promoters which would control expression of the FVIII transgene or the FIX transgene in different tissues or cells types, such as different hepatic tissues or cell types. Thus, in some embodiments, the lentiviral vector can comprise an endothelial specific promoter which would control expression of the FVIII transgene or the FIX transgene in hepatic endothelial tissue, or a hepatocyte specific promoter which would control expression of the FVIII transgene or the FIX transgene in hepatocytes, or both.

In some embodiments, the lentiviral vector comprises a tissue-specific promoter or tissue-specific promoters that control the expression of the FVIII transgene or the FIX transgene in tissues other than liver. In some embodiments, the isolated nucleic acid molecule is stably integrated into the genome of the target cell or target tissue, for example, in the genome of a hepatocyte or in the genome of a hepatic endothelial cell.

In some embodiments, the isolated nucleic acid molecule in a lentiviral vector of the present disclosure further comprises a heterologous nucleotide sequence encoding a heterologous amino acid sequence (e.g., a half-life extender). In some embodiments, the heterologous amino acid sequence is an immunoglobulin constant region or a portion thereof, XTEN, transferrin, albumin, or a PAS sequence. In some embodiments, the heterologous amino acid sequence is linked to the N-terminus or the C-terminus of the amino acid sequence encoded by the nucleotide sequence, or inserted between two amino acids in the amino acid sequence encoded by the nucleotide sequence at one or more insertion site selected from Table 2. Heterologous nucleotide sequences are further described herein.

In some embodiments, the polypeptide with FVIII activity is a human FVIII. In some embodiments, the polypeptide with FVIII activity is a full length FVIII. In some embodiments, the polypeptide with FVIII activity is a B domain deleted FVIII.

In some embodiments, the polypeptide with FIX activity is a human FIX. In some embodiments, the polypeptide with FIX activity is a full length FIX. In some embodiments, the polypeptide with FIX activity is a variant of human FIX. In certain embodiments, the polypeptide with FIX activity is a R338L variant of human FIX. In certain embodiments, the polypeptide with FIX activity is the Padua variant.

The lentiviral vectors disclosed herein can be used in vivo in a mammal, e.g., a human patient, using a gene therapy approach to treatment of a bleeding disease or disorder selected from the group consisting of a bleeding coagulation disorder, hemarthrosis, muscle bleed, oral bleed, hemorrhage, hemorrhage into muscles, oral hemorrhage, trauma, trauma capitis, gastrointestinal bleeding, intracranial hemorrhage, intra-abdominal hemorrhage, intrathoracic hemorrhage, bone fracture, central nervous system bleeding, bleeding in the retropharyngeal space, bleeding in the retroperitoneal space, and bleeding in the illiopsoas sheath would be therapeutically beneficial. In one embodiment, the bleeding disease or disorder is hemophilia. In another embodiment, the bleeding disease or disorder is hemophilia A. In another embodiment, the blooding disease or disorder is hemophilia B.

In some embodiments, target cells (e.g., hepatocytes) are treated in vitro with the lentiviral vectors disclosed herein before being administered to the patient. In certain embodiments, target cells (e.g., hepatocytes) are treated in vitro with the lentiviral vectors disclosed herein before being administered to the patient. In yet another embodiment, cells from the patient (e.g., hepatocytes) are treated ex vivo with the lentiviral vectors disclosed herein before being administered to the patient.

In some embodiments, plasma FVIII activity post administration of a lentiviral vectors disclosed herein (administered, e.g., at 10¹⁰ TU/kg or lower, 10⁹ TU/kg or lower, or 10⁸ TU/kg or lower) is increased by at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, or at least about 300%, relative to physiologically normal circulating FVIII levels.

In some embodiments, plasma FIX activity post administration of a lentiviral vectors disclosed herein (administered, e.g., at 10¹⁰ TU/kg or lower, 10⁹ TU/kg or lower, or 10⁸ TU/kg or lower) is increased by at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, or at least about 300%, relative to physiologically normal circulating FIX levels.

In one embodiment, the plasma FVIII activity post administration of a lentiviral vector of the present disclosure is increased by at least about 3,000% to about 5,000% relative to physiologically normal circulating FVIII levels. In some embodiments, post administration of a lentiviral vector comprising a codon-optimized gene encoding polypeptides with Factor VIII (FVIII) activity described herein, plasma FVIII activity is increased by at least about 10-fold, at least about 20-fold, at least about 30-fold, at least about 40-fold, at least about 50-fold, at least about 60-fold, at least about 70-fold, at least about 80-fold, at least about 90-fold, at least about 100-fold, at least about 110-fold, at least about 120-fold, at least about 130-fold, at least about 140-fold, at least about 150-fold, at least about 160-fold, at least about 170-fold, at least about 180-fold, at least about 190-fold, or at least about 200-fold relative to a subject administered a control lentiviral vector or a control nucleic acid molecule.

In one embodiment, the plasma FIX activity post administration of a lentiviral vector of the present disclosure is increased by at least about 3,000% to about 5,000% relative to physiologically normal circulating FIX levels. In some embodiments, post administration of a lentiviral vector comprising a codon-optimized gene encoding polypeptides with Factor IX (FIX) activity described herein, plasma FIX activity is increased by at least about 10-fold, at least about 20-fold, at least about 30-fold, at least about 40-fold, at least about 50-fold, at least about 60-fold, at least about 70-fold, at least about 80-fold, at least about 90-fold, at least about 100-fold, at least about 110-fold, at least about 120-fold, at least about 130-fold, at least about 140-fold, at least about 150-fold, at least about 160-fold, at least about 170-fold, at least about 180-fold, at least about 190-fold, or at least about 200-fold relative to a subject administered a control lentiviral vector or a control nucleic acid molecule.

The present disclosure also provides methods of treating, preventing. Or ameliorating a hemostatic disorder (e.g., a bleeding disorder such as hemophilia A or hemophilia B) in a subject in need thereof comprising administering to the subject a therapeutically effective amount of a lentiviral vector comprising an isolated nucleic acid molecule comprising a nucleotide sequence encoding a polypeptide with FVIII activity or a polypeptide with FIX activity.

The treatment, amelioration, and prevention by the lentiviral vector of the present disclosure can be a bypass therapy. The subject receiving bypass therapy can have already developed an inhibitor to a clotting factor, e.g., FVIII or FIX, or is subject to developing a clotting factor inhibitor.

The lentiviral vectors of the present disclosure treat or prevent a hemostatic disorder by promoting the formation of a fibrin clot. The polypeptide having FVIII or FIX activity encoded by the nucleic acid molecule of the disclosure can activate a member of a coagulation cascade. The clotting factor can be a participant in the extrinsic pathway, the intrinsic pathway or both.

The lentiviral vectors of the present disclosure can be used to treat hemostatic disorders known to be treatable with FVIII or FIX. The hemostatic disorders that can be treated using methods of the disclosure include, but are not limited to, hemophilia A, hemophilia B, von Willebrand's disease, Factor XI deficiency (PTA deficiency), Factor XII deficiency, as well as deficiencies or structural abnormalities in fibrinogen, prothrombin, Factor V, Factor VII, Factor X, or Factor XIII, hemarthrosis, muscle bleed, oral bleed, hemorrhage, hemorrhage into muscles, oral hemorrhage, trauma, trauma capitis, gastrointestinal bleeding, intracranial hemorrhage, intra-abdominal hemorrhage, intrathoracic hemorrhage, bone fracture, central nervous system bleeding, bleeding in the retropharyngeal space, bleeding in the retroperitoneal space, and bleeding in the illiopsoas sheath.

Compositions for administration to a subject include lentiviral vectors comprising nucleic acid molecules which comprise an optimized nucleotide sequence of the disclosure encoding a FVIII clotting factor or a FIX clotting factor (for gene therapy applications) as well as FVIII or FIX polypeptide molecules. In some embodiments, the composition for administration is a cell contacted with a lentiviral vector of the present disclosure, either in vivo, in vitro, or ex vivo.

In some embodiments, the hemostatic disorder is an inherited disorder. In one embodiment, the subject has hemophilia A. In other embodiments, the hemostatic disorder is the result of a deficiency in FVIII. In other embodiments, the hemostatic disorder can be the result of a defective FVIII clotting factor. In one embodiment, the subject has hemophilia B. In other embodiments, the hemostatic disorder is the result of a deficiency in FIX. In other embodiments, the hemostatic disorder can be the result of a defective FIX clotting factor.

In another embodiment, the hemostatic disorder can be an acquired disorder. The acquired disorder can result from an underlying secondary disease or condition. The unrelated condition can be, as an example, but not as a limitation, cancer, an autoimmune disease, or pregnancy. The acquired disorder can result from old age or from medication to treat an underlying secondary disorder (e.g., cancer chemotherapy).

The disclosure also relates to methods of treating a subject that does not have a hemostatic disorder or a secondary disease or condition resulting in acquisition of a hemostatic disorder. The disclosure thus relates to a method of treating a subject in need of a general hemostatic agent comprising administering a therapeutically effective amount of a lentiviral vector of the present disclosure. For example, in one embodiment, the subject in need of a general hemostatic agent is undergoing, or is about to undergo, surgery. The lentiviral vector of the disclosure can be administered prior to or after surgery as a prophylactic.

The lentiviral vector of the disclosure can be administered during or after surgery to control an acute bleeding episode. The surgery can include, but is not limited to, liver transplantation, liver resection, or stem cell transplantation.

In another embodiment, the lentiviral vector of the disclosure can be used to treat a subject having an acute bleeding episode who does not have a hemostatic disorder. The acute bleeding episode can result from severe trauma, e.g., surgery, an automobile accident, wound, laceration gun shot, or any other traumatic event resulting in uncontrolled bleeding.

The lentiviral vector can be used to prophylactically treat a subject with a hemostatic disorder. The lentiviral vector can also be used to treat an acute bleeding episode in a subject with a hemostatic disorder.

In another embodiment, the administration of a lentiviral vector disclosed herein and/or subsequent expression of FVIII protein or FIX protein does not induce an immune response in a subject. In some embodiments, the immune response comprises development of antibodies against FVIII or FIX. In some embodiments, the immune response comprises cytokine secretion. In some embodiments, the immune response comprises activation of B cells, T cells, or both B cells and T cells. In some embodiments, the immune response is an inhibitory immune response, wherein the immune response in the subject reduces the activity of the FVIII protein relative to the activity of the FVIII in a subject that has not developed an immune response. In certain embodiments, expression of FVIII protein by administering the lentiviral vector of the disclosure prevents an inhibitory immune response against the FVIII protein or the FVIII protein expressed from the isolated nucleic acid molecule or the lentiviral vector. In some embodiments, the immune response is an inhibitory immune response, wherein the immune response in the subject reduces the activity of the FIX protein relative to the activity of the FIX in a subject that has not developed an immune response. In certain embodiments, expression of FIX protein by administering the lentiviral vector of the disclosure prevents an inhibitory immune response against the FIX protein or the FIX protein expressed from the isolated nucleic acid molecule or the lentiviral vector.

In some embodiments, a lentiviral vector of the disclosure is administered in combination with at least one other agent that promotes hemostasis. Said other agent that promotes hemostasis in a therapeutic with demonstrated clotting activity. As an example, but not as a limitation, the hemostatic agent can include Factor V, Factor VII, Factor IX, Factor X, Factor XI, Factor XII, Factor XIII, prothrombin, or fibrinogen or activated forms of any of the preceding. The clotting factor or hemostatic agent can also include anti-fibrinolytic drugs, e.g., epsilon-amino-caproic acid, tranexamic acid.

In one embodiment of the disclosure, the composition (e.g., the lentiviral vector) is one in which the FVIII is present in activatable form when administered to a subject. In one embodiment of the disclosure, the composition (e.g., the lentiviral vector) is one in which the FIX is present in activatable form when administered to a subject. Such an activatable molecule can be activated in vivo at the site of clotting after administration to a subject.

The lentiviral vector of the disclosure can be administered intravenously, subcutaneously, intramuscularly, or via any mucosal surface, e.g., orally, sublingually, buccally, sublingually, nasally, rectally, vaginally or via pulmonary route. The lentiviral vector can be implanted within or linked to a biopolymer solid support that allows for the slow release of the vector to the desired site.

In one embodiment, the route of administration of the lentiviral vectors is parenteral. The term parenteral as used herein includes intravenous, intraarterial, intraperitoneal, intramuscular, subcutaneous, rectal or vaginal administration. The intravenous form of parenteral administration is preferred. While all these forms of administration are clearly contemplated as being within the scope of the disclosure, a form for administration would be a solution for injection, in particular for intravenous or intraarterial injection or drip. Usually, a suitable pharmaceutical composition for injection can comprise a buffer (e.g. acetate, phosphate or citrate buffer), a surfactant (e.g. polysorbate), optionally a stabilizer agent (e.g. human albumin), etc. However, in other methods compatible with the teachings herein, the lentiviral vector can be delivered directly to the site of the adverse cellular population thereby increasing the exposure of the diseased tissue to the therapeutic agent.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. In the subject disclosure, pharmaceutically acceptable carriers include, but are not limited to, 0.01-0.1M and preferably 0.05M phosphate buffer or 0.8% saline. Other common parenteral vehicles include sodium phosphate solutions, Ringer's dextrose, dextrose and sodium chloride, lactated Ringer's, or fixed oils. Intravenous vehicles include fluid and nutrient replenishers, electrolyte replenishers, such as those based on Ringer's dextrose, and the like. Preservatives and other additives can also be present such as for example, antimicrobials, antioxidants, chelating agents, and inert gases and the like.

More particularly, pharmaceutical compositions suitable for injectable use include sterile aqueous solutions (where water soluble) or dispersions and sterile powders for the extemporaneous preparation of sterile injectable solutions or dispersions. In such cases, the composition must be sterile and should be fluid to the extent that easy syringability exists. It should be stable under the conditions of manufacture and storage and will preferably be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants.

Prevention of the action of microorganisms can be achieved by various antibacterial and antifungal agents, for example, parabens, chlorobutanol, phenol, ascorbic acid, thimerosal and the like. In many cases, it will be preferable to include isotonic agents, for example, sugars, polyalcohols, such as mannitol, sorbitol, or sodium chloride in the composition. Prolonged absorption of the injectable compositions can be brought about by including in the composition an agent which delays absorption, for example, aluminum monostearate and gelatin.

In any case, sterile injectable solutions can be prepared by incorporating an active compound (e.g., a polypeptide by itself or in combination with other active agents) in the required amount in an appropriate solvent with one or a combination of ingredients enumerated herein, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yields a powder of an active ingredient plus any additional desired ingredient from a previously sterile-filtered solution thereof. The preparations for injections are processed, filled into containers such as ampoules, bags, bottles, syringes or vials, and sealed under aseptic conditions according to methods known in the art. Further, the preparations can be packaged and sold in the form of a kit. Such articles of manufacture will preferably have labels or package inserts indicating that the associated compositions are useful for treating a subject suffering from, or predisposed to clotting disorders.

The pharmaceutical composition can also be formulated for rectal administration as a suppository or retention enema, e.g., containing conventional suppository bases such as cocoa butter or other glycerides.

Effective doses of the compositions of the present disclosure, for the treatment of conditions vary depending upon many different factors, including means of administration, target site, physiological state of the patient, whether the patient is human or an animal, other medications administered, and whether treatment is prophylactic or therapeutic. Usually, the patient is a human, but non-human mammals including transgenic mammals can also be treated. Treatment dosages can be titrated using routine methods known to those of skill in the art to optimize safety and efficacy.

The lentiviral vector can be administered as a single dose or as multiple doses, wherein the multiple doses can be administered continuously or at specific timed intervals. In vitro assays can be employed to determine optimal dose ranges and/or schedules for administration. In vitro assays that measure clotting factor activity are known in the art. Additionally, effective doses can be extrapolated from dose-response curves obtained from animal models, e.g., a hemophiliac dog (Mount et al. 2002, Blood 99 (8): 2670).

Doses intermediate in the above ranges are also intended to be within the scope of the disclosure. Subjects can be administered such doses daily, on alternative days, weekly or according to any other schedule determined by empirical analysis. An exemplary treatment entails administration in multiple dosages over a prolonged period, for example, of at least six months.

The lentiviral vector of the disclosure can be administered on multiple occasions. Intervals between single dosages can be daily, weekly, monthly or yearly. Intervals can also be irregular as indicated by measuring blood levels of modified polypeptide or antigen in the patient. Dosage and frequency of the lentiviral vectors of the disclosure vary depending on the half-life of the FVIII polypeptide or the FIX polypeptide encoded by the transgene in the patient.

The dosage and frequency of administration of the lentiviral vectors of the disclosure can vary depending on whether the treatment is prophylactic or therapeutic. In prophylactic applications, compositions containing the lentiviral vector of the disclosure are administered to a patient not already in the disease state to enhance the patient's resistance or minimize effects of disease. Such an amount is defined to be a “prophylactic effective dose.” A relatively low dosage is administered at relatively infrequent intervals over a long period of time. Some patients continue to receive treatment for the rest of their lives.

The lentiviral vector of the disclosure can optionally be administered in combination with other agents that are effective in treating the disorder or condition in need of treatment (e.g., prophylactic or therapeutic).

As used herein, the administration of lentiviral vectors of the disclosure in conjunction or combination with an adjunct therapy means the sequential, simultaneous, coextensive, concurrent, concomitant or contemporaneous administration or application of the therapy and the disclosed polypeptides. Those skilled in the art will appreciate that the administration or application of the various components of the combined therapeutic regimen can be timed to enhance the overall effectiveness of the treatment. A skilled artisan (e.g., a physician) would be readily be able to discern effective combined therapeutic regimens without undue experimentation based on the selected adjunct therapy and the teachings of the instant specification.

It will further be appreciated that the lentiviral vectors of the disclosure can be used in conjunction or combination with an agent or agents (e.g., to provide a combined therapeutic regimen). Exemplary agents with which a lentiviral vector of the instant disclosure can be combined include agents that represent the current standard of care for a particular disorder being treated. Such agents can be chemical or biologic in nature. The term “biologic” or “biologic agent” refers to any pharmaceutically active agent made from living organisms and/or their products which is intended for use as a therapeutic.

The amount of agent to be used in combination with the lentiviral vectors of the instant disclosure can vary by subject or can be administered according to what is known in the art. See, e.g., Bruce A Chabner et al., Antineoplastic Agents, in GOODMAN & GILMAN'S THE PHARMACOLOGICAL BASIS OF THERAPEUTICS 1233-1287 ((Joel G. Hardman et al., eds., 9^{th} ed. 1996). In another embodiment, an amount of such an agent consistent with the standard of care is administered.

In certain embodiments, the lentiviral vectors of the present disclosure are administered in conjunction with an immunosuppressive, anti-allergic, or anti-inflammatory agent. These agents generally refer to substances that act to suppress or mask the immune system of the subject being treated herein. These agents include substances that suppress cytokine production, downregulate or suppress self-antigen expression, or mask the MHC antigens. Examples of such agents include 2-amino-6-aryl-5-substituted pyrimidines; azathioprine; cyclophosphamide; bromocryptine; danazol; dapsone; glutaraldehyde; anti-idiotypic antibodies for MHC antigens and MHC fragments; cyclosporin A; steroids such as glucocorticosteroids, e.g., prednisone, methylprednisolone, and dexamethasone; cytokine or cytokine receptor antagonists including anti-interferon-γ, -β, or -α antibodies, anti-tumor necrosis factor-α antibodies, anti-tumor necrosis factor-β antibodies, anti-interleukin-2 antibodies and anti-IL-2 receptor antibodies; anti-LFA-1 antibodies, including anti-CD11a and anti-CD18 antibodies; anti-L3T4 antibodies; heterologous anti-lymphocyte globulin; pan-T antibodies; soluble peptide containing a LFA-3 binding domain; streptokinase; TGF-β; streptodornase; FK506; RS-61443; deoxyspergualin; and rapamycin. In certain embodiments, the agent is an antihistamine. An “antihistamine” as used herein is an agent that antagonizes the physiological effect of histamine. Examples of antihistamines are chlorpheniramine, diphenhydramine, promethazine, cromolyn sodium, astemizole, azatadine maleate, bropheniramine maleate, carbinoxamine maleate, cetirizine hydrochloride, clemastine fumarate, cyproheptadine hydrochloride, dexbrompheniramine maleate, dexchlorpheniramine maleate, dimenhydrinate, diphenhydramine hydrochloride, doxylamine succinate, fexofendadine hydrochloride, terphenadine hydrochloride, hydroxyzine hydrochloride, loratidine, meclizine hydrochloride, tripelannamine citrate, tripelennamine hydrochloride, and triprolidine hydrochloride.

Immunosuppressive, anti-allergic, or anti-inflammatory agents may be incorporated into the lentiviral vector administration regimen. For example, administration of immunosuppressive or anti-inflammatory agents may commence prior to administration of the disclosed lentiviral vectors, and may continue with one or more doses thereafter. In certain embodiments, the immunosuppressive or anti-inflammatory agents are administered as premedication to the lentiviral vectors.

As previously discussed, the lentiviral vectors of the present disclosure, can be administered in a pharmaceutically effective amount for the in vivo treatment of clotting disorders. In this regard, it will be appreciated that the lentiviral vectors of the disclosure can be formulated to facilitate administration and promote stability of the active agent. Preferably, pharmaceutical compositions in accordance with the present disclosure comprise a pharmaceutically acceptable, non-toxic, sterile carrier such as physiological saline, non-toxic buffers, preservatives and the like. Of course, the pharmaceutical compositions of the present disclosure can be administered in single or multiple doses to provide for a pharmaceutically effective amount of the polypeptide.

A number of tests are available to assess the function of the coagulation system: activated partial thromboplastin time (aPTT) test, chromogenic assay, ROTEM® assay, prothrombin time (PT) test (also used to determine INR), fibrinogen testing (often by the Clauss method), platelet count, platelet function testing (often by PFA-100), TCT, bleeding time, mixing test (whether an abnormality corrects if the patient's plasma is mixed with normal plasma), coagulation factor assays, antiphosholipid antibodies, D-dimer, genetic tests (e.g., factor V Leiden, prothrombin mutation G20210A), dilute Russell's viper venom time (dRVVT), miscellaneous platelet function tests, thromboelastography (TEG or Sonoclot), thromboelastometry (TEM®, e.g, ROTEM®), or euglobulin lysis time (ELT).

The aPTT test is a performance indicator measuring the efficacy of both the “intrinsic” (also referred to the contact activation pathway) and the common coagulation pathways. This test is commonly used to measure clotting activity of commercially available recombinant clotting factors, e.g., FVIII or FIX. It is used in conjunction with prothrombin time (PT), which measures the extrinsic pathway.

ROTEM® analysis provides information on the whole kinetics of haemostasis: clotting time, clot formation, clot stability and lysis. The different parameters in thromboelastometry are dependent on the activity of the plasmatic coagulation system, platelet function, fibrinolysis, or many factors which influence these interactions. This assay can provide a complete view of secondary haemostasis.

### B.2. Tissue Specific Expression

In certain embodiments, it will be useful to include within the lentiviral vector one or more miRNA target sequences which, for example, are operably linked to the optimized FVIII transgene. Thus, the disclosure also provides at least one miRNA sequence target operably linked to the optimized FVIII or optimized FIX nucleotide sequence or otherwise inserted within a lentiviral vector. More than one copy of a miRNA target sequence included in the lentiviral vector can increase the effectiveness of the system.

Also included are different miRNA target sequences. For example, lentiviral vectors which express more than one transgene can have the transgene under control of more than one miRNA target sequence, which can be the same or different. The miRNA target sequences can be in tandem, but other arrangements are also included. The transgene expression cassette, containing miRNA target sequences, can also be inserted within the lentiviral vector in antisense orientation. Antisense orientation can be useful in the production of viral particles to avoid expression of gene products which can otherwise be toxic to the producer cells.

In other embodiments, the lentiviral vector comprises 1, 2, 3, 4, 5, 6, 7 or 8 copies of the same or different miRNA target sequence. In certain embodiments, the lentiviral vector does not include any miRNA target sequence. Choice of whether or not to include an miRNA target sequence (and how many) will be guided by known parameters such as the intended tissue target, the level of expression required, etc.

In one embodiment, the target sequence is an miR-223 target which has been reported to block expression most effectively in myeloid committed progenitors and at least partially in the more primitive HSPC. miR-223 target can block expression in differentiated myeloid cells including granulocytes, monocytes, macrophages, myeloid dendritic cells. miR-223 target can also be suitable for gene therapy applications relying on robust transgene expression in the lymphoid or erythroid lineage. miR-223 target can also block expression very effectively in human HSC.

In another embodiment, the target sequence is an miR142 target (tccataaagtaggaaacactaca (SEQ ID NO: 7)). In one embodiment, the lentiviral vector comprises 4 copies of miR-142 target sequences. In certain embodiments, the complementary sequence of hematopoietic-specific microRNAs, such as miR-142 (142T), is incorporated into the 3′ untranslated region of a lentiviral vector, making the transgene-encoding transcript susceptible to miRNA-mediated down-regulation. By this method, transgene expression can be prevented in hematopoietic-lineage antigen presenting cells (APC), while being maintained in non-hematopoietic cells (Brown et al., Nat Med 2006). This strategy can imposes a stringent post-transcriptional control on transgene expression and thus enables stable delivery and long-term expression of transgenes. In some embodiments, miR-142 regulation prevents immune-mediated clearance of transduced cells and/or induce antigen-specific Regulatory T cells (T regs) and mediate robust immunological tolerance to the transgene-encoded antigen.

In some embodiments, the target sequence is an miR181 target. Chen C-Z and Lodish H, Seminars in Immunology (2005) 17(2):155-165 discloses miR-181, a miRNA specifically expressed in B cells within mouse bone marrow (Chen and Lodish, 2005). It also discloses that some human miRNAs are linked to leukemias.

The target sequence can be fully or partially complementary to the miRNA. The term “fully complementary” means that the target sequence has a nucleic acid sequence which is 100% complementary to the sequence of the miRNA which recognizes it. The term “partially complementary” means that the target sequence is only in part complementary to the sequence of the miRNA which recognizes it, whereby the partially complementary sequence is still recognized by the miRNA. In other words, a partially complementary target sequence in the context of the present disclosure is effective in recognizing the corresponding miRNA and effecting prevention or reduction of transgene expression in cells expressing that miRNA. Examples of the miRNA target sequences are described at WO2007/000668, WO2004/094642, WO2010/055413, or WO2010/125471, which are incorporated herein by reference in their entireties.

### B.3. Heterologous Nucleotide Sequences

In some embodiments, the isolated nucleic acid molecule further comprises a heterologous nucleotide sequence. In some embodiments, the isolated nucleic acid molecule further comprises at least one heterologous nucleotide sequence. The heterologous nucleotide sequence can be linked with the FVIII or FIX coding sequences of the disclosure at the 5′ end, at the 3′ end, or inserted into the middle. Thus, in some embodiments, the heterologous amino acid sequence encoded by the heterologous nucleotide sequence is linked to the N-terminus or the C-terminus of the FVIII amino acid sequence or the FIX amino acid sequence encoded by the nucleotide sequence or inserted between two amino acids in the FVIII amino acid sequence or the FIX amino acid sequence. In some embodiments, the heterologous amino acid sequence can be inserted between two amino acids of an FVIII polypeptide at one or more insertion site selected from Table 2. In some embodiments, the heterologous amino acid sequence can be inserted within the FVIII polypeptide encoded by the nucleic acid molecule of the disclosure at any site disclosed in International Publication No. WO 2013/123457 A1 and WO 2015/106052 A1 or U.S. Publication No. 2015/0158929 A1, which are herein incorporated by reference in their entirety.

In some embodiments, the heterologous amino acid sequence encoded by the heterologous nucleotide sequence is inserted within the B domain or a fragment thereof. In some embodiments, the heterologous amino acid sequence is inserted within the FVIII immediately downstream of an amino acid corresponding to amino acid 745 of mature human FVIII (SEQ ID NO:4). In one particular embodiment, the FVIII comprises a deletion of amino acids 746-1646, corresponding to mature human FVIII (SEQ ID NO:4), and the heterologous amino acid sequence encoded by the heterologous nucleotide sequence is inserted immediately downstream of amino acid 745, corresponding to mature human FVIII (SEQ ID NO:4).

TABLE 2
Heterologous Moiety Insertion Sites

| Insertion | |
|---|---|
| Site | Domain |
| 3 | A1 |
| 18 | A1 |
| 22 | A1 |
| 26 | A1 |
| 40 | A1 |
| 60 | A1 |
| 65 | A1 |
| 81 | A1 |
| 116 | A1 |
| 119 | A1 |
| 130 | A1 |
| 188 | A1 |
| 211 | A1 |
| 216 | A1 |
| 220 | A1 |
| 224 | A1 |
| 230 | A1 |
| 333 | A1 |
| 336 | A1 |
| 339 | A1 |
| 375 | A2 |
| 378 | A2 |
| 399 | A2 |
| 403 | A2 |
| 409 | A2 |
| 416 | A2 |
| 442 | A2 |
| 487 | A2 |
| 490 | A2 |
| 494 | A2 |
| 500 | A2 |
| 518 | A2 |
| 599 | A2 |
| 603 | A2 |
| 713 | A2 |
| 745 | B |
| 1656 | a3 |
| | region |
| 1711 | A3 |
| 1720 | A3 |
| 1725 | A3 |
| 1749 | A3 |
| 1796 | A3 |
| 1802 | A3 |
| 1827 | A3 |
| 1861 | A3 |
| 1896 | A3 |
| 1900 | A3 |
| 1904 | A3 |
| 1905 | A3 |
| 1910 | A3 |
| 1937 | A3 |
| 2019 | A3 |
| 2068 | Cl |
| 2111 | Cl |
| 2120 | Cl |
| 2171 | C2 |
| 2188 | C2 |
| 2227 | C2 |
| 2332 | CT |

Note:
Insertion sites indicate the amino acid position corresponding to an amino acid position of mature human FVIII (SEQ ID NO: 4).

In other embodiments, the isolated nucleic acid molecule further comprise two, three, four, five, six, seven, or eight heterologous nucleotide sequences. In some embodiments, all the heterologous nucleotide sequences are identical. In some embodiments, at least one heterologous nucleotide sequence is different from the other heterologous nucleotide sequences. In some embodiments, the disclosure can comprise two, three, four, five, six, or more than seven heterologous nucleotide sequences in tandem.

In some embodiments, the heterologous nucleotide sequence encodes an amino acid sequence. In some embodiments, the amino acid sequence encoded by the heterologous nucleotide sequence is a heterologous moiety that can increase the half-life (a “half-life extender”) of an FVIII molecule.

In some embodiments, the heterologous moiety is a peptide or a polypeptide with either unstructured or structured characteristics that are associated with the prolongation of in vivo half-life when incorporated in a protein of the disclosure. Non-limiting examples include albumin, albumin fragments, Fc fragments of immunoglobulins, the C-terminal peptide (CTP) of the R subunit of human chorionic gonadotropin, a HAP sequence, an XTEN sequence, a transferrin or a fragment thereof, a PAS polypeptide, polyglycine linkers, polyserine linkers, albumin-binding moieties, or any fragments, derivatives, variants, or combinations of these polypeptides. In one particular embodiment, the heterologous amino acid sequence is an immunoglobulin constant region or a portion thereof, transferrin, albumin, or a PAS sequence.

In some aspects, a heterologous moiety includes von Willebrand factor or a fragment thereof. In other related aspects a heterologous moiety can include an attachment site (e.g., a cysteine amino acid) for a non-polypeptide moiety such as polyethylene glycol (PEG), hydroxyethyl starch (HES), polysialic acid, or any derivatives, variants, or combinations of these elements. In some aspects, a heterologous moiety comprises a cysteine amino acid that functions as an attachment site for a non-polypeptide moiety such as polyethylene glycol (PEG), hydroxyethyl starch (HES), polysialic acid, or any derivatives, variants, or combinations of these elements.

In one specific embodiment, a first heterologous nucleotide sequence encodes a first heterologous moiety that is a half-life extending molecule which is known in the art, and a second heterologous nucleotide sequence encodes a second heterologous moiety that can also be a half-life extending molecule which is known in the art. In certain embodiments, the first heterologous moiety (e.g., a first Fc moiety) and the second heterologous moiety (e.g., a second Fc moiety) are associated with each other to form a dimer. In one embodiment, the second heterologous moiety is a second Fc moiety, wherein the second Fc moiety is linked to or associated with the first heterologous moiety, e.g., the first Fc moiety. For example, the second heterologous moiety (e.g., the second Fc moiety) can be linked to the first heterologous moiety (e.g., the first Fc moiety) by a linker or associated with the first heterologous moiety by a covalent or non-covalent bond.

In some embodiments, the heterologous moiety is a polypeptide comprising, consisting essentially of, or consisting of at least about 10, at least about 100, at least about 200, at least about 300, at least about 400, at least about 500, at least about 600, at least about 700, at least about 800, at least about 900, at least about 1000, at least about 1100, at least about 1200, at least about 1300, at least about 1400, at least about 1500, at least about 1600, at least about 1700, at least about 1800, at least about 1900, at least about 2000, at least about 2500, at least about 3000, or at least about 4000 amino acids.

In other embodiments, the heterologous moiety is a polypeptide comprising, consisting essentially of, or consisting of about 100 to about 200 amino acids, about 200 to about 300 amino acids, about 300 to about 400 amino acids, about 400 to about 500 amino acids, about 500 to about 600 amino acids, about 600 to about 700 amino acids, about 700 to about 800 amino acids, about 800 to about 900 amino acids, or about 900 to about 1000 amino acids.

In certain embodiments, a heterologous moiety improves one or more pharmacokinetic properties of the FVIII or FIX protein without significantly affecting its biological activity or function.

In certain embodiments, a heterologous moiety increases the in vivo and/or in vitro half-life of the FVIII or FIX protein of the disclosure. In other embodiments, a heterologous moiety facilitates visualization or localization of the FVIII or FIX protein of the disclosure or a fragment thereof (e.g., a fragment comprising a heterologous moiety after proteolytic cleavage of the FVIII or FIX protein). Visualization and/or location of the FVIII or FIX protein of the disclosure or a fragment thereof can be in vivo, in vitro, ex vivo, or combinations thereof.

In other embodiments, a heterologous moiety increases stability of the FVIII or FIX protein of the disclosure or a fragment thereof (e.g., a fragment comprising a heterologous moiety after proteolytic cleavage of the FVIII or FIX protein). As used herein, the term “stability” refers to an art-recognized measure of the maintenance of one or more physical properties of the FVIII or FIX protein in response to an environmental condition (e.g., an elevated or lowered temperature). In certain aspects, the physical property can be the maintenance of the covalent structure of the FVIII or FIX protein (e.g., the absence of proteolytic cleavage, unwanted oxidation or deamidation). In other aspects, the physical property can also be the presence of the FVIII or FIX protein in a properly folded state (e.g., the absence of soluble or insoluble aggregates or precipitates).

In one aspect, the stability of the FVIII or FIX protein is measured by assaying a biophysical property of the FVIII or FIX protein, for example thermal stability, pH unfolding profile, stable removal of glycosylation, solubility, biochemical function (e.g., ability to bind to a protein, receptor or ligand), etc., and/or combinations thereof. In another aspect, biochemical function is demonstrated by the binding affinity of the interaction. In one aspect, a measure of protein stability is thermal stability, i.e., resistance to thermal challenge. Stability can be measured using methods known in the art, such as, HPLC (high performance liquid chromatography), SEC (size exclusion chromatography), DLS (dynamic light scattering), etc. Methods to measure thermal stability include, but are not limited to differential scanning calorimetry (DSC), differential scanning fluorimetry (DSF), circular dichroism (CD), and thermal challenge assay.

In certain aspects, a FVIII or FIX protein encoded by the nucleic acid molecule of the disclosure comprises at least one half-life extender, i.e., a heterologous moiety which increases the in vivo half-life of the FVIII or FIX protein with respect to the in vivo half-life of the corresponding FVIII or FIX protein lacking such heterologous moiety. In vivo half-life of a FVIII or FIX protein can be determined by any methods known to those of skill in the art, e.g., activity assays (chromogenic assay or one stage clotting aPTT assay), ELISA, ROTEM™, etc.

In some embodiments, the presence of one or more half-life extenders results in the half-life of the FVIII or FIX protein to be increased compared to the half-life of the corresponding protein lacking such one or more half-life extenders. The half-life of the FVIII or FIX protein comprising a half-life extender is at least about 1.5 times, at least about 2 times, at least about 2.5 times, at least about 3 times, at least about 4 times, at least about 5 times, at least about 6 times, at least about 7 times, at least about 8 times, at least about 9 times, at least about 10 times, at least about 11 times, or at least about 12 times longer than the in vivo half-life of the corresponding FVIII or FIX protein lacking such half-life extender.

In one embodiment, the half-life of the FVIII or FIX protein comprising a half-life extender is about 1.5-fold to about 20-fold, about 1.5 fold to about 15 fold, or about 1.5 fold to about 10 fold longer than the in vivo half-life of the corresponding protein lacking such half-life extender. In another embodiment, the half-life of FVIII or FIX protein comprising a half-life extender is extended about 2-fold to about 10-fold, about 2-fold to about 9-fold, about 2-fold to about 8-fold, about 2-fold to about 7-fold, about 2-fold to about 6-fold, about 2-fold to about 5-fold, about 2-fold to about 4-fold, about 2-fold to about 3-fold, about 2.5-fold to about 10-fold, about 2.5-fold to about 9-fold, about 2.5-fold to about 8-fold, about 2.5-fold to about 7-fold, about 2.5-fold to about 6-fold, about 2.5-fold to about 5-fold, about 2.5-fold to about 4-fold, about 2.5-fold to about 3-fold, about 3-fold to about 10-fold, about 3-fold to about 9-fold, about 3-fold to about 8-fold, about 3-fold to about 7-fold, about 3-fold to about 6-fold, about 3-fold to about 5-fold, about 3-fold to about 4-fold, about 4-fold to about 6 fold, about 5-fold to about 7-fold, or about 6-fold to about 8 fold as compared to the in vivo half-life of the corresponding protein lacking such half-life extender.

In other embodiments, the half-life of the FVIII or FIX protein comprising a half-life extender is at least about 17 hours, at least about 18 hours, at least about 19 hours, at least about 20 hours, at least about 21 hours, at least about 22 hours, at least about 23 hours, at least about 24 hours, at least about 25 hours, at least about 26 hours, at least about 27 hours, at least about 28 hours, at least about 29 hours, at least about 30 hours, at least about 31 hours, at least about 32 hours, at least about 33 hours, at least about 34 hours, at least about 35 hours, at least about 36 hours, at least about 48 hours, at least about 60 hours, at least about 72 hours, at least about 84 hours, at least about 96 hours, or at least about 108 hours.

In still other embodiments, the half-life of the FVIII or FIX protein comprising a half-life extender is about 15 hours to about two weeks, about 16 hours to about one week, about 17 hours to about one week, about 18 hours to about one week, about 19 hours to about one week, about 20 hours to about one week, about 21 hours to about one week, about 22 hours to about one week, about 23 hours to about one week, about 24 hours to about one week, about 36 hours to about one week, about 48 hours to about one week, about 60 hours to about one week, about 24 hours to about six days, about 24 hours to about five days, about 24 hours to about four days, about 24 hours to about three days, or about 24 hours to about two days.

In some embodiments, the average half-life per subject of the FVIII or FIX protein comprising a half-life extender is about 15 hours, about 16 hours, about 17 hours, about 18 hours, about 19 hours, about 20 hours, about 21 hours, about 22 hours, about 23 hours, about 24 hours (1 day), about 25 hours, about 26 hours, about 27 hours, about 28 hours, about 29 hours, about 30 hours, about 31 hours, about 32 hours, about 33 hours, about 34 hours, about 35 hours, about 36 hours, about 40 hours, about 44 hours, about 48 hours (2 days), about 54 hours, about 60 hours, about 72 hours (3 days), about 84 hours, about 96 hours (4 days), about 108 hours, about 120 hours (5 days), about six days, about seven days (one week), about eight days, about nine days, about 10 days, about 11 days, about 12 days, about 13 days, or about 14 days.

One or more half-life extenders can be fused to C-terminus or N-terminus of FVIII or FIX or inserted within FVIII or FIX.

B.3.a. An Immunoglobulin Constant Region or a Portion Thereof

In another aspect, a heterologous moiety comprises one or more immunoglobulin constant regions or portions thereof (e.g., an Fc region). In one embodiment, an isolated nucleic acid molecule of the disclosure further comprises a heterologous nucleic acid sequence that encodes an immunoglobulin constant region or a portion thereof. In some embodiments, the immunoglobulin constant region or portion thereof is an Fc region.

An immunoglobulin constant region is comprised of domains denoted CH (constant heavy) domains (CH1, CH2, etc.). Depending on the isotype, (i.e. IgG, IgM, IgA IgD, or IgE), the constant region can be comprised of three or four CH domains. Some isotypes (e.g. IgG) constant regions also contain a hinge region. See Janeway et al. 2001, Immunobiology, Garland Publishing, N.Y., N.Y.

An immunoglobulin constant region or a portion thereof for producing the FVIII protein of the present disclosure can be obtained from a number of different sources. In one embodiment, an immunoglobulin constant region or a portion thereof is derived from a human immunoglobulin. It is understood, however, that the immunoglobulin constant region or a portion thereof can be derived from an immunoglobulin of another mammalian species, including for example, a rodent (e.g., a mouse, rat, rabbit, guinea pig) or non-human primate (e.g., chimpanzee, macaque) species. Moreover, the immunoglobulin constant region or a portion thereof can be derived from any immunoglobulin class, including IgM, IgG, IgD, IgA and IgE, and any immunoglobulin isotype, including IgG1, IgG2, IgG3 and IgG4. In one embodiment, the human isotype IgG1 is used.

A variety of the immunoglobulin constant region gene sequences (e.g., human constant region gene sequences) are available in the form of publicly accessible deposits. Constant region domains sequence can be selected having a particular effector function (or lacking a particular effector function) or with a particular modification to reduce immunogenicity. Many sequences of antibodies and antibody-encoding genes have been published and suitable Ig constant region sequences (e.g., hinge, CH2, and/or CH3 sequences, or portions thereof) can be derived from these sequences using art recognized techniques. The genetic material obtained using any of the foregoing methods can then be altered or synthesized to obtain polypeptides of the present disclosure. It will further be appreciated that the scope of this disclosure encompasses alleles, variants and mutations of constant region DNA sequences.

The sequences of the immunoglobulin constant region or a portion thereof can be cloned, e.g., using the polymerase chain reaction and primers which are selected to amplify the domain of interest. To clone a sequence of the immunoglobulin constant region or a portion thereof from an antibody, mRNA can be isolated from hybridoma, spleen, or lymph cells, reverse transcribed into DNA, and antibody genes amplified by PCR. PCR amplification methods are described in detail in U.S. Pat. Nos. 4,683,195; 4,683,202; 4,800,159; 4,965,188; and in, e.g., “PCR Protocols: A Guide to Methods and Applications” Innis et al. eds., Academic Press, San Diego, CA (1990); Ho et al. 1989. Gene 77:51; Horton et al. 1993. Methods Enzymol. 217:270). PCR can be initiated by consensus constant region primers or by more specific primers based on the published heavy and light chain DNA and amino acid sequences. PCR also can be used to isolate DNA clones encoding the antibody light and heavy chains. In this case the libraries can be screened by consensus primers or larger homologous probes, such as mouse constant region probes. Numerous primer sets suitable for amplification of antibody genes are known in the art (e.g., 5′ primers based on the N-terminal sequence of purified antibodies (Benhar and Pastan. 1994. Protein Engineering 7:1509); rapid amplification of cDNA ends (Ruberti, F. et al. 1994. J. Immunol. Methods 173:33); antibody leader sequences (Larrick et al. 1989 Biochem. Biophys. Res. Commun. 160:1250). The cloning of antibody sequences is further described in Newman et al., U.S. Pat. No. 5,658,570, filed Jan. 25, 1995, which is incorporated by reference herein.

An immunoglobulin constant region used herein can include all domains and the hinge region or portions thereof. In one embodiment, the immunoglobulin constant region or a portion thereof comprises CH2 domain, CH3 domain, and a hinge region, i.e., an Fc region or an FcRn binding partner.

As used herein, the term “Fc region” is defined as the portion of a polypeptide which corresponds to the Fc region of native Ig, i.e., as formed by the dimeric association of the respective Fc domains of its two heavy chains. A native Fc region forms a homodimer with another Fc region. In contrast, the term “genetically-fused Fc region” or “single-chain Fc region” (scFc region), as used herein, refers to a synthetic dimeric Fc region comprised of Fc domains genetically linked within a single polypeptide chain (i.e., encoded in a single contiguous genetic sequence). See International Publication No. WO 2012/006635, incorporated herein by reference in its entirety.

In one embodiment, the “Fc region” refers to the portion of a single Ig heavy chain beginning in the hinge region just upstream of the papain cleavage site (i.e. residue 216 in IgG, taking the first residue of heavy chain constant region to be 114) and ending at the C-terminus of the antibody. Accordingly, a complete Fc region comprises at least a hinge domain, a CH2 domain, and a CH3 domain.

An immunoglobulin constant region or a portion thereof can be an FcRn binding partner. FcRn is active in adult epithelial tissues and expressed in the lumen of the intestines, pulmonary airways, nasal surfaces, vaginal surfaces, colon and rectal surfaces (U.S. Pat. No. 6,485,726). An FcRn binding partner is a portion of an immunoglobulin that binds to FcRn.

The FcRn receptor has been isolated from several mammalian species including humans. The sequences of the human FcRn, monkey FcRn, rat FcRn, and mouse FcRn are known (Story et al. 1994, J. Exp. Med. 180:2377). The FcRn receptor binds IgG (but not other immunoglobulin classes such as IgA, IgM, IgD, and IgE) at relatively low pH, actively transports the IgG transcellularly in a luminal to serosal direction, and then releases the IgG at relatively higher pH found in the interstitial fluids. It is expressed in adult epithelial tissue (U.S. Pat. Nos. 6,485,726, 6,030,613, 6,086,875; WO 03/077834; US2003-0235536A1) including lung and intestinal epithelium (Israel et al. 1997, Immunology 92:69) renal proximal tubular epithelium (Kobayashi et al. 2002, Am. J. Physiol. Renal Physiol. 282:F358) as well as nasal epithelium, vaginal surfaces, and biliary tree surfaces.

FcRn binding partners useful in the present disclosure encompass molecules that can be specifically bound by the FcRn receptor including whole IgG, the Fc fragment of IgG, and other fragments that include the complete binding region of the FcRn receptor. The region of the Fc portion of IgG that binds to the FcRn receptor has been described based on X-ray crystallography (Burmeister et al. 1994, Nature 372:379). The major contact area of the Fc with the FcRn is near the junction of the CH2 and CH3 domains. Fc-FcRn contacts are all within a single Ig heavy chain. The FcRn binding partners include whole IgG, the Fc fragment of IgG, and other fragments of IgG that include the complete binding region of FcRn. The major contact sites include amino acid residues 248, 250-257, 272, 285, 288, 290-291, 308-311, and 314 of the CH2 domain and amino acid residues 385-387, 428, and 433-436 of the CH3 domain. References made to amino acid numbering of immunoglobulins or immunoglobulin fragments, or regions, are all based on Kabat et al. 1991, Sequences of Proteins of Immunological Interest, U.S. Department of Public Health, Bethesda, Md.

Fc regions or FcRn binding partners bound to FcRn can be effectively shuttled across epithelial barriers by FcRn, thus providing a non-invasive means to systemically administer a desired therapeutic molecule. Additionally, fusion proteins comprising an Fc region or an FcRn binding partner are endocytosed by cells expressing the FcRn. But instead of being marked for degradation, these fusion proteins are recycled out into circulation again, thus increasing the in vivo half-life of these proteins. In certain embodiments, the portions of immunoglobulin constant regions are an Fc region or an FcRn binding partner that typically associates, via disulfide bonds and other non-specific interactions, with another Fc region or another FcRn binding partner to form dimers and higher order multimers.

Two FcRn receptors can bind a single Fc molecule. Crystallographic data suggest that each FcRn molecule binds a single polypeptide of the Fc homodimer. In one embodiment, linking the FcRn binding partner, e.g., an Fc fragment of an IgG, to a biologically active molecule provides a means of delivering the biologically active molecule orally, buccally, sublingually, rectally, vaginally, as an aerosol administered nasally or via a pulmonary route, or via an ocular route. In another embodiment, the FVIII protein can be administered invasively, e.g., subcutaneously, intravenously.

An FcRn binding partner region is a molecule or portion thereof that can be specifically bound by the FcRn receptor with consequent active transport by the FcRn receptor of the Fc region. Specifically bound refers to two molecules forming a complex that is relatively stable under physiologic conditions. Specific binding is characterized by a high affinity and a low to moderate capacity as distinguished from nonspecific binding which usually has a low affinity with a moderate to high capacity. Typically, binding is considered specific when the affinity constant KA is higher than 10⁶ M⁻¹, or higher than 108 M⁻¹. If necessary, non-specific binding can be reduced without substantially affecting specific binding by varying the binding conditions. The appropriate binding conditions such as concentration of the molecules, ionic strength of the solution, temperature, time allowed for binding, concentration of a blocking agent (e.g., serum albumin, milk casein), etc., can be optimized by a skilled artisan using routine techniques.

In certain embodiments, a FVIII protein encoded by the nucleic acid molecule of the disclosure comprises one or more truncated Fc regions that are nonetheless sufficient to confer Fc receptor (FcR) binding properties to the Fc region. For example, the portion of an Fc region that binds to FcRn (i.e., the FcRn binding portion) comprises from about amino acids 282-438 of IgG1, EU numbering (with the primary contact sites being amino acids 248, 250-257, 272, 285, 288, 290-291, 308-311, and 314 of the CH2 domain and amino acid residues 385-387, 428, and 433-436 of the CH3 domain. Thus, an Fc region of the disclosure can comprise or consist of an FcRn binding portion. FcRn binding portions can be derived from heavy chains of any isotype, including IgG1, IgG2, IgG3 and IgG4. In one embodiment, an FcRn binding portion from an antibody of the human isotype IgG1 is used. In another embodiment, an FcRn binding portion from an antibody of the human isotype IgG4 is used.

The Fc region can be obtained from a number of different sources. In one embodiment, an Fc region of the polypeptide is derived from a human immunoglobulin. It is understood, however, that an Fc moiety can be derived from an immunoglobulin of another mammalian species, including for example, a rodent (e.g., a mouse, rat, rabbit, guinea pig) or non-human primate (e.g., chimpanzee, macaque) species. Moreover, the polypeptide of the Fc domains or portions thereof can be derived from any immunoglobulin class, including IgM, IgG, IgD, IgA and IgE, and any immunoglobulin isotype, including IgG1, IgG2, IgG3 and IgG4. In another embodiment, the human isotype IgG1 is used.

In certain embodiments, the Fc variant confers a change in at least one effector function imparted by an Fc moiety comprising said wild-type Fc domain (e.g., an improvement or reduction in the ability of the Fc region to bind to Fc receptors (e.g. FcγRI, FcγRII, or FcγRIII) or complement proteins (e.g. C1q), or to trigger antibody-dependent cytotoxicity (ADCC), phagocytosis, or complement-dependent cytotoxicity (CDCC)). In other embodiments, the Fc variant provides an engineered cysteine residue.

The Fc region of the disclosure can employ art-recognized Fc variants which are known to impart a change (e.g., an enhancement or reduction) in effector function and/or FcR or FcRn binding. Specifically, an Fc region of the disclosure can include, for example, a change (e.g., a substitution) at one or more of the amino acid positions disclosed in International PCT Publications WO88/07089A1, WO96/14339A1, WO98/05787A1, WO98/23289A1, WO99/51642A1, WO99/58572A1, WO00/09560A2, WO00/32767A1, WO00/42072A2, WO02/44215A2, WO02/060919A2, WO03/074569A2, WO04/016750A2, WO04/029207A2, WO04/035752A2, WO04/063351A2, WO04/074455A2, WO04/099249A2, WO05/040217A2, WO04/044859, WO05/070963A1, WO05/077981A2, WO05/092925A2, WO05/123780A2, WO06/019447A1, WO06/047350A2, and WO06/085967A2; US Patent Publication Nos. US2007/0231329, US2007/0231329, US2007/0237765, US2007/0237766, US2007/0237767, US2007/0243188, US2007/0248603, US2007/0286859, US2008/0057056; or U.S. Pat. Nos. 5,648,260; 5,739,277; 5,834,250; 5,869,046; 6,096,871; 6,121,022; 6,194,551; 6,242,195; 6,277,375; 6,528,624; 6,538,124; 6,737,056; 6,821,505; 6,998,253; 7,083,784; 7,404,956, and 7,317,091, each of which is incorporated by reference herein. In one embodiment, the specific change (e.g., the specific substitution of one or more amino acids disclosed in the art) can be made at one or more of the disclosed amino acid positions. In another embodiment, a different change at one or more of the disclosed amino acid positions (e.g., the different substitution of one or more amino acid position disclosed in the art) can be made.

The Fc region or FcRn binding partner of IgG can be modified according to well recognized procedures such as site directed mutagenesis and the like to yield modified IgG or Fc fragments or portions thereof that will be bound by FcRn. Such modifications include modifications remote from the FcRn contact sites as well as modifications within the contact sites that preserve or even enhance binding to the FcRn. For example, the following single amino acid residues in human IgG1 Fc (Fc□1) can be substituted without significant loss of Fc binding affinity for FcRn: P238A, S239A, K246A, K248A, D249A, M252A, T256A, E258A, T260A, D265A, S267A, H268A, E269A, D270A, E272A, L274A, N276A, Y278A, D280A, V282A, E283A, H285A, N286A, T289A, K290A, R292A, E293A, E294A, Q295A, Y296F, N297A, S298A, Y300F, R301A, V303A, V305A, T307A, L309A, Q311A, D312A, N315A, K317A, E318A, K320A, K322A, S324A, K326A, A327Q, P329A, A330Q, P331A, E333A, K334A, T335A, S337A, K338A, K340A, Q342A, R344A, E345A, Q347A, R355A, E356A, M358A, T359A, K360A, N361A, Q362A, Y373A, S375A, D376A, A378Q, E380A, E382A, S383A, N384A, Q386A, E388A, N389A, N390A, Y391F, K392A, L398A, S400A, D401A, D413A, K414A, R416A, Q418A, Q419A, N421A, V422A, S424A, E430A, N434A, T437A, Q438A, K439A, S440A, S444A, and K447A, where for example P238A represents wild type proline substituted by alanine at position number 238. As an example, a specific embodiment incorporates the N297A mutation, removing a highly conserved N-glycosylation site. In addition to alanine other amino acids can be substituted for the wild type amino acids at the positions specified above. Mutations can be introduced singly into Fc giving rise to more than one hundred Fc regions distinct from the native Fc. Additionally, combinations of two, three, or more of these individual mutations can be introduced together, giving rise to hundreds more Fc regions.

Certain of the above mutations can confer new functionality upon the Fc region or FcRn binding partner. For example, one embodiment incorporates N297A, removing a highly conserved N-glycosylation site. The effect of this mutation is to reduce immunogenicity, thereby enhancing circulating half-life of the Fc region, and to render the Fc region incapable of binding to FcγRI, FcγRIIA, FcγRIIB, and FcγRIIIA, without compromising affinity for FcRn (Routledge et al. 1995, Transplantation 60:847; Friend et al. 1999, Transplantation 68:1632; Shields et al. 1995, J. Biol. Chem. 276:6591). As a further example of new functionality arising from mutations described above affinity for FcRn can be increased beyond that of wild type in some instances. This increased affinity can reflect an increased “on” rate, a decreased “off” rate or both an increased “on” rate and a decreased “off” rate. Examples of mutations believed to impart an increased affinity for FcRn include, but not limited to, T256A, T307A, E380A, and N434A (Shields et al. 2001, J. Biol. Chem. 276:6591).

Additionally, at least three human Fc gamma receptors appear to recognize a binding site on IgG within the lower hinge region, generally amino acids 234-237. Therefore, another example of new functionality and potential decreased immunogenicity can arise from mutations of this region, as for example by replacing amino acids 233-236 of human IgG1 “ELLG” (SEQ ID NO:8) to the corresponding sequence from IgG2 “PVA” (with one amino acid deletion). It has been shown that FcγRI, FcγRII, and FcγRIII, which mediate various effector functions will not bind to IgG1 when such mutations have been introduced. Ward and Ghetie 1995, Therapeutic Immunology 2:77 and Armour et al. 1999, Eur. J. Immunol. 29:2613.

In another embodiment, the immunoglobulin constant region or a portion thereof comprises an amino acid sequence in the hinge region or a portion thereof that forms one or more disulfide bonds with a second immunoglobulin constant region or a portion thereof. The second immunoglobulin constant region or a portion thereof can be linked to a second polypeptide, bringing the FVIII protein and the second polypeptide together. In some embodiments, the second polypeptide is an enhancer moiety. As used herein, the term “enhancer moiety” refers to a molecule, fragment thereof or a component of a polypeptide which is capable of enhancing the procoagulant activity of FVIII. The enhancer moiety can be a cofactor, such as soluble tissue factor (sTF), or a procoagulant peptide. Thus, upon activation of FVIII, the enhancer moiety is available to enhance FVIII activity.

In certain embodiments, a FVIII protein encoded by a nucleic acid molecule of the disclosure comprises an amino acid substitution to an immunoglobulin constant region or a portion thereof (e.g., Fc variants), which alters the antigen-independent effector functions of the Ig constant region, in particular the circulating half-life of the protein.

B.3.b. scFc Regions

In another aspect, a heterologous moiety comprises a scFc (single chain Fc) region. In one embodiment, an isolated nucleic acid molecule of the disclosure further comprises a heterologous nucleic acid sequence that encodes a scFc region. The scFc region comprises at least two immunoglobulin constant regions or portions thereof (e.g., Fc moieties or domains (e.g., 2, 3, 4, 5, 6, or more Fc moieties or domains)) within the same linear polypeptide chain that are capable of folding (e.g., intramolecularly or intermolecularly folding) to form one functional scFc region which is linked by an Fc peptide linker. For example, in one embodiment, a polypeptide of the disclosure is capable of binding, via its scFc region, to at least one Fc receptor (e.g., an FcRn, an FcγR receptor (e.g., FcγRIII), or a complement protein (e.g., C1q)) in order to improve half-life or trigger an immune effector function (e.g., antibody-dependent cytotoxicity (ADCC), phagocytosis, or complement-dependent cytotoxicity (CDCC) and/or to improve manufacturability).

B.3.c. CTP

In another aspect, a heterologous moiety comprises one C-terminal peptide (CTP) of the β subunit of human chorionic gonadotropin or fragment, variant, or derivative thereof. One or more CTP peptides inserted into a recombinant protein is known to increase the in vivo half-life of that protein. See, e.g., U.S. Pat. No. 5,712,122, incorporated by reference herein in its entirety.

Exemplary CTP peptides include DPRFQDSSSSKAPPPSLPSPSRLPGPSDTPIL (SEQ ID NO:9) or SSSSKAPPPSLPSPSRLPGPSDTPILPQ (SEQ ID NO:10). See, e.g., U.S. Patent Application Publication No. US 2009/0087411 A1, incorporated by reference.

B.3.d. XTEN Sequence

In some embodiments, a heterologous moiety comprises one or more XTEN sequences, fragments, variants, or derivatives thereof. As used here “XTEN sequence” refers to extended length polypeptides with non-naturally occurring, substantially non-repetitive sequences that are composed mainly of small hydrophilic amino acids, with the sequence having a low degree or no secondary or tertiary structure under physiologic conditions. As a heterologous moiety, XTENs can serve as a half-life extension moiety. In addition, XTEN can provide desirable properties including but are not limited to enhanced pharmacokinetic parameters and solubility characteristics.

The incorporation of a heterologous moiety comprising an XTEN sequence into a protein of the disclosure can confer to the protein one or more of the following advantageous properties: conformational flexibility, enhanced aqueous solubility, high degree of protease resistance, low immunogenicity, low binding to mammalian receptors, or increased hydrodynamic (or Stokes) radii.

In certain aspects, an XTEN sequence can increase pharmacokinetic properties such as longer in vivo half-life or increased area under the curve (AUC), so that a protein of the disclosure stays in vivo and has procoagulant activity for an increased period of time compared to a protein with the same but without the XTEN heterologous moiety.

In some embodiments, the XTEN sequence useful for the disclosure is a peptide or a polypeptide having greater than about 20, 30, 40, 50, 60, 70, 80, 90, 100, 150, 200, 250, 300, 350, 400, 450, 500, 550, 600, 650, 700, 750, 800, 850, 900, 950, 1000, 1200, 1400, 1600, 1800, or 2000 amino acid residues. In certain embodiments, XTEN is a peptide or a polypeptide having greater than about 20 to about 3000 amino acid residues, greater than 30 to about 2500 residues, greater than 40 to about 2000 residues, greater than 50 to about 1500 residues, greater than 60 to about 1000 residues, greater than 70 to about 900 residues, greater than 80 to about 800 residues, greater than 90 to about 700 residues, greater than 100 to about 600 residues, greater than 110 to about 500 residues, or greater than 120 to about 400 residues. In one particular embodiment, the XTEN comprises an amino acid sequence of longer than 42 amino acids and shorter than 144 amino acids in length.

The XTEN sequence of the disclosure can comprise one or more sequence motif of 5 to 14 (e.g., 9 to 14) amino acid residues or an amino acid sequence at least 80%, 90%, 91%, 92%, 93%, 94%, 95%, 96%, 97%, 98%, or 99% identical to the sequence motif, wherein the motif comprises, consists essentially of, or consists of 4 to 6 types of amino acids (e.g., 5 amino acids) selected from the group consisting of glycine (G), alanine (A), serine (S), threonine (T), glutamate (E) and proline (P). See US 2010-0239554 A1.

In some embodiments, the XTEN comprises non-overlapping sequence motifs in which about 80%, or at least about 85%, or at least about 90%, or about 91%, or about 92%, or about 93%, or about 94%, or about 95%, or about 96%, or about 97%, or about 98%, or about 99% or about 100% of the sequence consists of multiple units of non-overlapping sequences selected from a single motif family selected from Table 3, resulting in a family sequence.

As used herein, “family” means that the XTEN has motifs selected only from a single motif category from Table 3; i.e., AD, AE, AF, AG, AM, AQ, BC, or BD XTEN, and that any other amino acids in the XTEN not from a family motif are selected to achieve a needed property, such as to permit incorporation of a restriction site by the encoding nucleotides, incorporation of a cleavage sequence, or to achieve a better linkage to FVIII. In some embodiments of XTEN families, an XTEN sequence comprises multiple units of non-overlapping sequence motifs of the AD motif family, or of the AE motif family, or of the AF motif family, or of the AG motif family, or of the AM motif family, or of the AQ motif family, or of the BC family, or of the BD family, with the resulting XTEN exhibiting the range of homology described above. In other embodiments, the XTEN comprises multiple units of motif sequences from two or more of the motif families of Table 3.

These sequences can be selected to achieve desired physical/chemical characteristics, including such properties as net charge, hydrophilicity, lack of secondary structure, or lack of repetitiveness that are conferred by the amino acid composition of the motifs, described more fully below. In the embodiments hereinabove described in this paragraph, the motifs incorporated into the XTEN can be selected and assembled using the methods described herein to achieve an XTEN of about 36 to about 3000 amino acid residues.

TABLE 3
XTEN Sequence Motifs of 12 Amino Acids and
Motif Families

| Motif | | |
|---|---|---|
| Family* | MOTIF SEQUENCE | SEQ ID NO: |
| AD | GESPGGSSGSES | 11 |
| AD | GSEGSSGPGESS | 12 |
| AD | GSSESGSSEGGP | 13 |
| AD | GSGGEPSESGSS | 14 |
| AE, AM | GSPAGSPTSTEE | 15 |
| AE, AM, AQ | GSEPATSGSETP | 16 |
| AE, AM, AQ | GTSESATPESGP | 17 |
| AE, AM, AQ | GTSTEPSEGSAP | 18 |
| AF, AM | GSTSESPSGTAP | 19 |
| AF, AM | GTSTPESGSASP | 20 |
| AF, AM | GTSPSGESSTAP | 21 |
| AF, AM | GSTSSTAESPGP | 22 |
| AG, AM | GTPGSGTASSSP | 23 |
| AG, AM | GSSTPSGATGSP | 24 |
| AG, AM | GSSPSASTGTGP | 25 |
| AG, AM | GASPGTSSTGSP | 26 |
| AQ | GEPAGSPTSTSE | 27 |
| AQ | GTGEPSSTPASE | 28 |
| AQ | GSGPSTESAPTE | 29 |
| AQ | GSETPSGPSETA | 30 |
| AQ | GPSETSTSEPGA | 31 |
| AQ | GSPSEPTEGTSA | 32 |
| BC | GSGASEPTSTEP | 33 |
| BC | GSEPATSGTEPS | 34 |
| BC | GTSEPSTSEPGA | 35 |
| BC | GTSTEPSEPGSA | 36 |
| BD | GSTAGSETSTEA | 37 |
| BD | GSETATSGSETA | 38 |
| BD | GTSESATSESGA | 39 |
| BD | GTSTEASEGSAS | 40 |

*Denotes individual motif sequences that, when used together in various permutations, results in a “family sequence”

Examples of XTEN sequences that can be used as heterologous moieties in chimeric proteins of the disclosure are disclosed, e.g., in U.S. Patent Publication Nos. 2010/0239554 A1, 2010/0323956 A1, 2011/0046060 A1, 2011/0046061 A1, 2011/0077199 A1, or 2011/0172146 A1, or International Patent Publication Nos. WO 2010/091122 A1, WO 2010/144502 A2, WO 2010/144508 A1, WO 2011/028228 A1, WO 2011/028229 A1, or WO 2011/028344 A2, each of which is incorporated by reference herein in its entirety.

XTEN can have varying lengths for insertion into or linkage to FVIII. In one embodiment, the length of the XTEN sequence(s) is chosen based on the property or function to be achieved in the fusion protein. Depending on the intended property or function, XTEN can be short or intermediate length sequence or longer sequence that can serve as carriers. In certain embodiments, the XTEN includes short segments of about 6 to about 99 amino acid residues, intermediate lengths of about 100 to about 399 amino acid residues, and longer lengths of about 400 to about 1000 and up to about 3000 amino acid residues. Thus, the XTEN inserted into or linked to FVIII can have lengths of about 6, about 12, about 36, about 40, about 42, about 72, about 96, about 144, about 288, about 400, about 500, about 576, about 600, about 700, about 800, about 864, about 900, about 1000, about 1500, about 2000, about 2500, or up to about 3000 amino acid residues in length. In other embodiments, the XTEN sequences is about 6 to about 50, about 50 to about 100, about 100 to 150, about 150 to 250, about 250 to 400, about 400 to about 500, about 500 to about 900, about 900 to 1500, about 1500 to 2000, or about 2000 to about 3000 amino acid residues in length.

The precise length of an XTEN inserted into or linked to FVIII can vary without adversely affecting the activity of the FVIII. In one embodiment, one or more of the XTENs used herein have 42 amino acids, 72 amino acids, 144 amino acids, 288 amino acids, 576 amino acids, or 864 amino acids in length and can be selected from one or more of the XTEN family sequences; i.e., AD, AE, AF, AG, AM, AQ, BC or BD.

In some embodiments, the XTEN sequence used in the disclosure is at least 60%, 70%, 80%, 85%, 90%, 91%, 92%, 93%, 94%, 95%, 96%, 97%, 98%, 99%, or 100% identical to a sequence selected from the group consisting of AE42, AG42, AE48, AM48, AE72, AG72, AE108, AG108, AE144, AF144, AG144, AE180, AG180, AE216, AG216, AE252, AG252, AE288, AG288, AE324, AG324, AE360, AG360, AE396, AG396, AE432, AG432, AE468, AG468, AE504, AG504, AF504, AE540, AG540, AF540, AD576, AE576, AF576, AG576, AE612, AG612, AE624, AE648, AG648, AG684, AE720, AG720, AE756, AG756, AE792, AG792, AE828, AG828, AD836, AE864, AF864, AG864, AM875, AE912, AM923, AM1318, BC864, BD864, AE948, AE1044, AE1140, AE1236, AE1332, AE1428, AE1524, AE1620, AE1716, AE1812, AE1908, AE2004A, AG948, AG1044, AG1140, AG1236, AG1332, AG1428, AG1524, AG1620, AG1716, AG1812, AG1908, AG2004, and any combination thereof. See US 2010-0239554 A1. In one particular embodiment, the XTEN comprises AE42, AE72, AE144, AE288, AE576, AE864, AG 42, AG72, AG144, AG288, AG576, AG864, or any combination thereof.

Exemplary XTEN sequences that can be used as heterologous moieties in chimeric protein of the disclosure include XTEN AE42-4 (SEQ ID NO:41), XTEN 144-2A (SEQ ID NO:42), XTEN A144-3B (SEQ ID NO:43), XTEN AE144-4A (SEQ ID NO:44), XTEN AE144-5A (SEQ ID NO:45), XTEN AE144-6B (SEQ ID NO:46), XTEN AG144-1 (SEQ ID NO:47), XTEN AG144-A (SEQ ID NO:48), XTEN AG144-B (SEQ ID NO:49), XTEN AG144-C(SEQ ID NO:50), and XTEN AG144-F (SEQ ID NO:51). In one particular embodiment, the XTEN is encoded by SEQ ID NO:52.

In some embodiments, less than 100% of amino acids of an XTEN are selected from glycine (G), alanine (A), serine (S), threonine (T), glutamate (E) and proline (P), or less than 100% of the sequence consists of the sequence motifs from Table 3 or an XTEN sequence provided herein. In such embodiments, the remaining amino acid residues of the XTEN are selected from any of the other 14 natural L-amino acids, but can be preferentially selected from hydrophilic amino acids such that the XTEN sequence contains at least about 90%, 91%, 92%, 93%, 94%, 95%, 96%, 97%, 98%, or at least about 99% hydrophilic amino acids.

The content of hydrophobic amino acids in the XTEN utilized in the conjugation constructs can be less than 5%, or less than 2%, or less than 1% hydrophobic amino acid content.

Hydrophobic residues that are less favored in construction of XTEN include tryptophan, phenylalanine, tyrosine, leucine, isoleucine, valine, and methionine. Additionally, XTEN sequences can contain less than 5% or less than 4% or less than 3% or less than 2% or less than 1% or none of the following amino acids: methionine (for example, to avoid oxidation), or asparagine and glutamine (to avoid desamidation).

The one or more XTEN sequences can be inserted at the C-terminus or at the N-terminus of the amino acid sequence encoded by the nucleotide sequence or inserted between two amino acids in the amino acid sequence encoded by the nucleotide sequence. For example, the XTEN can be inserted between two amino acids at one or more insertion site selected from Table 2. Examples of sites within FVIII that are permissible for XTEN insertion can be found in, e.g., International Publication No. WO 2013/123457 A1 or U.S. Publication No. 2015/0158929 A1, which are herein incorporated by reference in their entirety.

B.3.e. Albumin or Fragment, Derivative, or Variant Thereof

In some embodiments, a heterologous moiety comprises albumin or a functional fragment thereof. Human serum albumin (HSA, or HA), a protein of 609 amino acids in its full-length form, is responsible for a significant proportion of the osmotic pressure of serum and also functions as a carrier of endogenous and exogenous ligands. The term “albumin” as used herein includes full-length albumin or a functional fragment, variant, derivative, or analog thereof. Examples of albumin or the fragments or variants thereof are disclosed in US Pat. Publ. Nos. 2008/0194481A1, 2008/0004206 A1, 2008/0161243 A1, 2008/0261877 A1, or 2008/0153751 A1 or PCT Appl. Publ. Nos. WO 2008/033413 A2, WO 2009/058322 A1, or WO 2007/021494 A2, which are incorporated herein by reference in their entireties.

In one embodiment, the FVIII protein encoded by a nucleic acid molecule of the disclosure comprises albumin, a fragment, or a variant thereof which is further linked to a second heterologous moiety selected from the group consisting of an immunoglobulin constant region or portion thereof (e.g., an Fc region), a PAS sequence, HES, and PEG.

### B.3.f Albumin-Binding Moiety

In certain embodiments, the heterologous moiety is an albumin-binding moiety, which comprises an albumin-binding peptide, a bacterial albumin-binding domain, an albumin-binding antibody fragment, or any combinations thereof.

For example, the albumin-binding protein can be a bacterial albumin-binding protein, an antibody or an antibody fragment including domain antibodies (see U.S. Pat. No. 6,696,245). An albumin-binding protein, for example, can be a bacterial albumin-binding domain, such as the one of streptococcal protein G (Konig, T. and Skerra, A. (1998) J. Immunol. Methods 218, 73-83). Other examples of albumin-binding peptides that can be used as conjugation partner are, for instance, those having a Cys-Xaa₁-Xaa₂-Xaa₃-Xaa₄-Cys (SEQ ID NO:52) consensus sequence, wherein Xaa₁ is Asp, Asn, Ser, Thr, or Trp; Xaa₂ is Asn, Gln, H is, Ile, Leu, or Lys; Xaa₃ is Ala, Asp, Phe, Trp, or Tyr; and Xaa₄ is Asp, Gly, Leu, Phe, Ser, or Thr as described in US Patent Application Publication No. 2003/0069395 or Dennis et al. (Dennis et al. (2002) J. Biol. Chem. 277, 35035-35043).

Domain 3 from streptococcal protein G, as disclosed by Kraulis et al., FEBS Lett. 378:190-194 (1996) and Linhult et al., Protein Sci. 11:206-213 (2002) is an example of a bacterial albumin-binding domain. Examples of albumin-binding peptides include a series of peptides having the core sequence DICLPRWGCLW (SEQ ID NO:54). See, e.g., Dennis et al., J. Biol. Chem. 2002, 277: 35035-35043 (2002). Examples of albumin-binding antibody fragments are disclosed in Muller and Kontermann, Curr. Opin. Mol. Ther. 9:319-326 (2007); Roovers et al., Cancer Immunol. Immunother. 56:303-317 (2007), and Holt et al., Prot. Eng. Design Sci., 21:283-288 (2008), which are incorporated herein by reference in their entireties. An example of such albumin-binding moiety is 2-(3-maleimidopropanamido)-6-(4-(4-iodophenyl)butanamido) hexanoate (“Albu” tag) as disclosed by Trussel et al., Bioconjugate Chem. 20:2286-2292 (2009).

Fatty acids, in particular long chain fatty acids (LCFA) and long chain fatty acid-like albumin-binding compounds can be used to extend the in vivo half-life of FVIII proteins of the disclosure. An example of a LCFA-like albumin-binding compound is 16-(1-(3-(9-(((2,5-dioxopyrrolidin-1-yloxy) carbonyloxy)-methyl)-7-sulfo-9H-fluoren-2-ylamino)-3-oxopropyl)-2,5-dioxopyrrolidin-3-ylthio) hexadecanoic acid (see, e.g., WO 2010/140148).

B.3. g. PAS Sequence

In other embodiments, the heterologous moiety is a PAS sequence. A PAS sequence, as used herein, means an amino acid sequence comprising mainly alanine and serine residues or comprising mainly alanine, serine, and proline residues, the amino acid sequence forming random coil conformation under physiological conditions. Accordingly, the PAS sequence is a building block, an amino acid polymer, or a sequence cassette comprising, consisting essentially of, or consisting of alanine, serine, and proline which can be used as a part of the heterologous moiety in the chimeric protein. Yet, the skilled person is aware that an amino acid polymer also can form random coil conformation when residues other than alanine, serine, and proline are added as a minor constituent in the PAS sequence.

The term “minor constituent” as used herein means that amino acids other than alanine, serine, and proline can be added in the PAS sequence to a certain degree, e.g., up to about 12%, i.e., about 12 of 100 amino acids of the PAS sequence, up to about 10%, i.e. about 10 of 100 amino acids of the PAS sequence, up to about 9%, i.e., about 9 of 100 amino acids, up to about 8%, i.e., about 8 of 100 amino acids, about 6%, i.e., about 6 of 100 amino acids, about 5%, i.e., about 5 of 100 amino acids, about 4%, i.e., about 4 of 100 amino acids, about 3%, i.e., about 3 of 100 amino acids, about 2%, i.e., about 2 of 100 amino acids, about 1%, i.e., about 1 of 100 of the amino acids. The amino acids different from alanine, serine and proline can be selected from the group consisting of Arg, Asn, Asp, Cys, Gln, Glu, Gly, His, Ile, Leu, Lys, Met, Phe, Thr, Trp, Tyr, and Val.

Under physiological conditions, the PAS sequence stretch forms a random coil conformation and thereby can mediate an increased in vivo and/or in vitro stability to the FVIII protein. Since the random coil domain does not adopt a stable structure or function by itself, the biological activity mediated by the FVIII protein is essentially preserved. In other embodiments, the PAS sequences that form random coil domain are biologically inert, especially with respect to proteolysis in blood plasma, immunogenicity, isoelectric point/electrostatic behavior, binding to cell surface receptors or internalisation, but are still biodegradable, which provides clear advantages over synthetic polymers such as PEG.

Non-limiting examples of the PAS sequences forming random coil conformation comprise an amino acid sequence selected from the group consisting of ASPAAPAPASPAAPAPSAPA (SEQ ID NO:55), AAPASPAPAAPSAPAPAAPS (SEQ ID NO:56), APSSPSPSAPSSPSPASPSS (SEQ ID NO:57), APSSPSPSAPSSPSPASPS (SEQ ID NO:58), SSPSAPSPSSPASPSPSSPA (SEQ ID NO:59), AASPAAPSAPPAAASPAAPSAPPA (SEQ ID NO:60) and ASAAAPAAASAAASAPSAAA (SEQ ID NO:61) or any combinations thereof. Additional examples of PAS sequences are known from, e.g., US Pat. Publ. No. 2010/0292130 A1 and PCT Appl. Publ. No. WO 2008/155134 A1.

B.3.h. HAP Sequence

In certain embodiments, the heterologous moiety is a glycine-rich homo-amino-acid polymer (HAP). The HAP sequence can comprise a repetitive sequence of glycine, which has at least 50 amino acids, at least 100 amino acids, 120 amino acids, 140 amino acids, 160 amino acids, 180 amino acids, 200 amino acids, 250 amino acids, 300 amino acids, 350 amino acids, 400 amino acids, 450 amino acids, or 500 amino acids in length. In one embodiment, the HAP sequence is capable of extending half-life of a moiety fused to or linked to the HAP sequence. Non-limiting examples of the HAP sequence includes, but are not limited to (Gly)ₙ, (Gly₄Ser)ₙ or S(Gly₄Ser)ₙ, wherein n is 1, 2, 3, 4, 5, 6, 7, 8, 9, 10, 11, 12, 13, 14, 15, 16, 17, 18, 19, or 20. In one embodiment, n is 20, 21, 22, 23, 24, 25, 26, 26, 28, 29, 30, 31, 32, 33, 34, 35, 36, 37, 38, 39, or 40. In another embodiment, n is 50, 60, 70, 80, 90, 100, 110, 120, 130, 140, 150, 160, 170, 180, 190, or 200.

B.3.i. Transferrin or Fragment Thereof

In certain embodiments, the heterologous moiety is transferrin or a fragment thereof. Any transferrin can be used to make the FVIII proteins of the disclosure. As an example, wild-type human TF (TF) is a 679 amino acid protein, of approximately 75 KDa (not accounting for glycosylation), with two main domains, N (about 330 amino acids) and C (about 340 amino acids), which appear to originate from a gene duplication. See GenBank accession numbers NM001063, XM002793, M12530, XM039845, XM 039847 and S95936 (www.ncbi.nlm.nih.gov/), all of which are herein incorporated by reference in their entirety. Transferrin comprises two domains, N domain and C domain. N domain comprises two subdomains, N1 domain and N2 domain, and C domain comprises two subdomains, C1 domain and C2 domain.

In one embodiment, the transferrin heterologous moiety includes a transferrin splice variant. In one example, a transferrin splice variant can be a splice variant of human transferrin, e.g., Genbank Accession AAA61140. In another embodiment, the transferrin portion of the chimeric protein includes one or more domains of the transferrin sequence, e.g., N domain, C domain, N1 domain, N2 domain, C1 domain, C2 domain or any combinations thereof.

B.3. j. Clearance Receptors

In certain embodiments, the heterologous moiety is a clearance receptor, fragment, variant, or derivative thereof. LRP1 is a 600 kDa integral membrane protein that is implicated in the receptor-mediate clearance of a variety of proteins, such as Factor X. See, e.g., Narita et al., Blood 91:555-560 (1998).

B.3.k. Von Willebrand Factor or Fragments Thereof

In certain embodiments, the heterologous moiety is von Willebrand Factor (VWF) or one or more fragments thereof.

VWF (also known as F8VWF) is a large multimeric glycoprotein present in blood plasma and produced constitutively in endothelium (in the Weibel-Palade bodies), megakaryocytes (α-granules of platelets), and subendothelian connective tissue. The basic VWF monomer is a 2813 amino acid protein. Every monomer contains a number of specific domains with a specific function, the D′ and D3 domains (which together bind to Factor VIII), the A1 domain (which binds to platelet GPIb-receptor, heparin, and/or possibly collagen), the A3 domain (which binds to collagen), the C1 domain (in which the RGD domain binds to platelet integrin αIIbβ3 when this is activated), and the “cysteine knot” domain at the C-terminal end of the protein (which VWF shares with platelet-derived growth factor (PDGF), transforming growth factor-β (TGFβ) and β-human chorionic gonadotropin (PHCG)).

The 2813 monomer amino acid sequence for human VWF is reported as Accession Number NP000543.2 in Genbank. The nucleotide sequence encoding the human VWF is reported as Accession Number NM000552.3 in Genbank. SEQ ID NO:62 is the amino acid sequence reported in Genbank Accession Number NM000552.3. The D′ domain includes amino acids 764 to 866 of SEQ ID NO:62. The D3 domain includes amino acids 867 to 1240 of SEQ ID NO:62.

In plasma, 95-98% of FVIII circulates in a tight non-covalent complex with full-length VWF. The formation of this complex is important for the maintenance of appropriate plasma levels of FVIIII in vivo. Lenting et al., Blood. 92(11): 3983-96 (1998); Lenting et al., J. Thromb. Haemost. 5(7): 1353-60 (2007). When FVIII is activated due to proteolysis at positions 372 and 740 in the heavy chain and at position 1689 in the light chain, the VWF bound to FVIII is removed from the activated FVIII.

In certain embodiments, the heterologous moiety is full length von Willebrand Factor. In other embodiments, the heterologous moiety is a von Willebrand Factor fragment. As used herein, the term “VWF fragment” or “VWF fragments” used herein means any VWF fragments that interact with FVIII and retain at least one or more properties that are normally provided to FVIII by full-length VWF, e.g., preventing premature activation to FVIIIa, preventing premature proteolysis, preventing association with phospholipid membranes that could lead to premature clearance, preventing binding to FVIII clearance receptors that can bind naked FVIII but not VWF-bound FVIII, and/or stabilizing the FVIII heavy chain and light chain interactions. In a specific embodiment, the heterologous moiety is a (VWF) fragment comprising a D′ domain and a D3 domain of VWF. The VWF fragment comprising the D′ domain and the D3 domain can further comprise a VWF domain selected from the group consisting of an A1 domain, an A2 domain, an A3 domain, a D1 domain, a D2 domain, a D4 domain, a B1 domain, a B2 domain, a B3 domain, a C1 domain, a C2 domain, a CK domain, one or more fragments thereof, and any combinations thereof. Additional examples of the polypeptide having FVIII activity fused to the VWF fragment are disclosed in U.S. provisional patent application No. 61/667,901, filed Jul. 3, 2012, and U.S. Publication No. 2015/0023959 A1, which are both incorporated herein by reference in its entirety.

B.3.l. Linker Moieties

In certain embodiments, the heterologous moiety is a peptide linker.

As used herein, the terms “peptide linkers” or “linker moieties” refer to a peptide or polypeptide sequence (e.g., a synthetic peptide or polypeptide sequence) which connects two domains in a linear amino acid sequence of a polypeptide chain.

In some embodiments, heterologous nucleotide sequences encoding peptide linkers can be inserted between the optimized FVIII polynucleotide sequences of the disclosure and a heterologous nucleotide sequence encoding, for example, one of the heterologous moieties described above, such as albumin. Peptide linkers can provide flexibility to the chimeric polypeptide molecule. Linkers are not typically cleaved, however such cleavage can be desirable. In one embodiment, these linkers are not removed during processing.

A type of linker which can be present in a chimeric protein of the disclosure is a protease cleavable linker which comprises a cleavage site (i.e., a protease cleavage site substrate, e.g., a factor XIa, Xa, or thrombin cleavage site) and which can include additional linkers on either the N-terminal of C-terminal or both sides of the cleavage site. These cleavable linkers when incorporated into a construct of the disclosure result in a chimeric molecule having a heterologous cleavage site.

In one embodiment, an FVIII polypeptide encoded by a nucleic acid molecule of the instant disclosure comprises two or more Fc domains or moieties linked via a cscFc linker to form an Fc region comprised in a single polypeptide chain. The cscFc linker is flanked by at least one intracellular processing site, i.e., a site cleaved by an intracellular enzyme. Cleavage of the polypeptide at the at least one intracellular processing site results in a polypeptide which comprises at least two polypeptide chains.

Other peptide linkers can optionally be used in a construct of the disclosure, e.g., to connect an FVIII protein to an Fc region. Some exemplary linkers that can be used in connection with the disclosure include, e.g., polypeptides comprising GlySer amino acids described in more detail below.

In one embodiment, the peptide linker is synthetic, i.e., non-naturally occurring. In one embodiment, a peptide linker includes peptides (or polypeptides) (which can or cannot be naturally occurring) which comprise an amino acid sequence that links or genetically fuses a first linear sequence of amino acids to a second linear sequence of amino acids to which it is not naturally linked or genetically fused in nature. For example, in one embodiment the peptide linker can comprise non-naturally occurring polypeptides which are modified forms of naturally occurring polypeptides (e.g., comprising a mutation such as an addition, substitution or deletion). In another embodiment, the peptide linker can comprise non-naturally occurring amino acids. In another embodiment, the peptide linker can comprise naturally occurring amino acids occurring in a linear sequence that does not occur in nature. In still another embodiment, the peptide linker can comprise a naturally occurring polypeptide sequence.

For example, in certain embodiments, a peptide linker can be used to fuse identical Fc moieties, thereby forming a homodimeric scFc region. In other embodiments, a peptide linker can be used to fuse different Fc moieties (e.g. a wild-type Fc moiety and an Fc moiety variant), thereby forming a heterodimeric scFc region.

In another embodiment, a peptide linker comprises or consists of a gly-ser linker. In one embodiment, a scFc or cscFc linker comprises at least a portion of an immunoglobulin hinge and a gly-ser linker. As used herein, the term “gly-ser linker” refers to a peptide that consists of glycine and serine residues. In certain embodiments, said gly-ser linker can be inserted between two other sequences of the peptide linker. In other embodiments, a gly-ser linker is attached at one or both ends of another sequence of the peptide linker. In yet other embodiments, two or more gly-ser linker are incorporated in series in a peptide linker. In one embodiment, a peptide linker of the disclosure comprises at least a portion of an upper hinge region (e.g., derived from an IgG1, IgG2, IgG3, or IgG4 molecule), at least a portion of a middle hinge region (e.g., derived from an IgG1, IgG2, IgG3, or IgG4 molecule) and a series of gly/ser amino acid residues.

Peptide linkers of the disclosure are at least one amino acid in length and can be of varying lengths. In one embodiment, a peptide linker of the disclosure is from about 1 to about 50 amino acids in length. As used in this context, the term “about” indicates+/− two amino acid residues. Since linker length must be a positive integer, the length of from about 1 to about 50 amino acids in length, means a length of from 1-3 to 48-52 amino acids in length. In another embodiment, a peptide linker of the disclosure is from about 10 to about 20 amino acids in length. In another embodiment, a peptide linker of the disclosure is from about 15 to about 50 amino acids in length. In another embodiment, a peptide linker of the disclosure is from about 20 to about 45 amino acids in length. In another embodiment, a peptide linker of the disclosure is from about 15 to about 35 or about 20 to about 30 amino acids in length. In another embodiment, a peptide linker of the disclosure is from about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, 100, 500, 1000, or 2000 amino acids in length. In one embodiment, a peptide linker of the disclosure is 20 or 30 amino acids in length.

In some embodiments, the peptide linker can comprise at least two, at least three, at least four, at least five, at least 10, at least 20, at least 30, at least 40, at least 50, at least 60, at least 70, at least 80, at least 90, or at least 100 amino acids. In other embodiments, the peptide linker can comprise at least 200, at least 300, at least 400, at least 500, at least 600, at least 700, at least 800, at least 900, or at least 1,000 amino acids. In some embodiments, the peptide linker can comprise at least about 10, 20, 30, 40, 50, 60, 70, 80, 90, 100, 150, 200, 300, 400, 500, 600, 700, 800, 900, 1000, 1100, 1200, 1300, 1400, 1500, 1600, 1700, 1800, 1900, or 2000 amino acids. The peptide linker can comprise 1-5 amino acids, 1-10 amino acids, 1-20 amino acids, 10-50 amino acids, 50-100 amino acids, 100-200 amino acids, 200-300 amino acids, 300-400 amino acids, 400-500 amino acids, 500-600 amino acids, 600-700 amino acids, 700-800 amino acids, 800-900 amino acids, or 900-1000 amino acids.

Peptide linkers can be introduced into polypeptide sequences using techniques known in the art. Modifications can be confirmed by DNA sequence analysis. Plasmid DNA can be used to transform host cells for stable production of the polypeptides produced.

B.3.m. Monomer-Dimer Hybrids

In some embodiments, the isolated nucleic acid molecules of the disclosure which further comprise a heterologous nucleotide sequence encode a monomer-dimer hybrid molecule comprising FVIII.

The term “monomer-dimer hybrid” used herein refers to a chimeric protein comprising a first polypeptide chain and a second polypeptide chain, which are associated with each other by a disulfide bond, wherein, e.g., the first chain comprises Factor VIII and a first Fc region and the second chain comprises, consists essentially of, or consists of a second Fc region without the FVIII. The monomer-dimer hybrid construct thus is a hybrid comprising a monomer aspect having only one clotting factor and a dimer aspect having two Fc regions.

B.3.n. Expression Control Element

In some embodiments, the nucleic acid molecule or vector of the disclosure further comprises at least one expression control sequence. A expression control sequences as used herein is any regulatory nucleotide sequence, such as a promoter sequence or promoter-enhancer combination, which facilitates the efficient transcription and translation of the coding nucleic acid to which it is operably linked. For example, the isolated nucleic acid molecule of the disclosure can be operably linked to at least one transcription control sequence.

The gene expression control sequence can, for example, be a mammalian or viral promoter, such as a constitutive or inducible promoter. Constitutive mammalian promoters include, but are not limited to, the promoters for the following genes: hypoxanthine phosphoribosyl transferase (HPRT), adenosine deaminase, pyruvate kinase, beta-actin promoter, and other constitutive promoters. Exemplary viral promoters which function constitutively in eukaryotic cells include, for example, promoters from the cytomegalovirus (CMV), simian virus (e.g., SV40), papilloma virus, adenovirus, human immunodeficiency virus (HIV), Rous sarcoma virus, cytomegalovirus, the long terminal repeats (LTR) of Moloney leukemia virus, and other retroviruses, and the thymidine kinase promoter of herpes simplex virus.

Other constitutive promoters are known to those of ordinary skill in the art. The promoters useful as gene expression sequences of the disclosure also include inducible promoters. Inducible promoters are expressed in the presence of an inducing agent. For example, the metallothionein promoter is induced to promote transcription and translation in the presence of certain metal ions. Other inducible promoters are known to those of ordinary skill in the art.

In one embodiment, the disclosure includes expression of a transgene under the control of a tissue specific promoter and/or enhancer. In another embodiment, the promoter or other expression control sequence selectively enhances expression of the transgene in liver cells. Examples of liver specific promoters include, but are not limited to, a mouse thyretin promoter (mTTR), an endogenous human factor VIII (F8) promoter, an endogenous human factor IX (F9) promoter, human alpha-1-antitrypsin promoter (hAAT), human albumin minimal promoter, and mouse albumin promoter. In a particular embodiment, the promoter comprises a mTTR promoter. The mTTR promoter is described in R. H. Costa et al., 1986, Mol. Cell. Biol. 6:4697. The F8 promoter is described in Figueiredo and Brownlee, 1995, J. Biol. Chem. 270:11828-11838. In certain embodiments, the promoter comprises any of the mTTR promoters (e.g., mTTR202 promoter, mTTR202opt promoter, mTTR482 promoter) as disclosed in U.S. patent publication no. US2019/0048362, which is incorporated by reference herein in its entirety.

Expression levels can be further enhanced to achieve therapeutic efficacy using one or more enhancers. One or more enhancers can be provided either alone or together with one or more promoter elements. Typically, the expression control sequence comprises a plurality of enhancer elements and a tissue specific promoter. In one embodiment, an enhancer comprises one or more copies of the α-1-microglobulin/bikunin enhancer (Rouet et al., 1992, J. Biol. Chem. 267:20765-20773; Rouet et al., 1995, Nucleic Acids Res. 23:395-404; Rouet et al., 1998, Biochem. J. 334:577-584; Ill et al., 1997, Blood Coagulation Fibrinolysis 8:S23-S30). In another embodiment, an enhancer is derived from liver specific transcription factor binding sites, such as EBP, DBP, HNF1, HNF3, HNF4, HNF6, with Enh1, comprising HNF1, (sense)-HNF3, (sense)-HNF4, (antisense)-HNF1, (antisense)-HNF6, (sense)-EBP, (antisense)-HNF4 (antisense).

In a particular example, a promoter useful for the disclosure comprises SEQ ID NO:63 (i.e., ET promoter), which is also known as GenBank No. AY661265. See also Vigna et al., Molecular Therapy 11(5):763 (2005). Examples of other suitable vectors and gene regulatory elements are described in WO 02/092134, EP1395293, or U.S. Pat. Nos. 6,808,905, 7,745,179, or 7,179,903, which are incorporated by reference herein in their entireties.

In general, the expression control sequences shall include, as necessary, 5′ non-transcribing and 5′ non-translating sequences involved with the initiation of transcription and translation, respectively, such as a TATA box, capping sequence, CAAT sequence, and the like. Especially, such 5′ non-transcribing sequences will include a promoter region which includes a promoter sequence for transcriptional control of the operably joined coding nucleic acid. The gene expression sequences optionally include enhancer sequences or upstream activator sequences as desired.

### EXAMPLES

### Example 1—Recombinant Lentiviral Vector (LV) Preparation

Lentiviral vector drug product stability was assessed by exposure of vector to various stress conditions (e.g. freezing and thawing (F/T), elevated temperature (37° C.), agitation) and monitoring changes over time. Methods of determining stability included: ddPCR for functional titer, p24 ELISA for p24 concentration, and NanoSight for particle size distribution and particle concentration. Functional titer is a cell-based assay (HEK293) whereby LV is incubated with cells, allowed to integrate into cellular genome, extracted, and DNA is measured with ddPCR. The ELISA based p24 method is a kit-based method (Invitrogen) where the viral capsid protein, p24, is measured and related to a total particle concentration. NanoSight is a method that uses Brownian motion of the particles to evaluate size and concentration of LV particles in solution.

Functional titer is a dose defining parameter and is a key metric. It provides information about whether LVs are stable and can therefore integrate their payload into cells (related to the efficacy of the drug and mechanism of action). Functional titer is a dose defining criterion.

### Vector Production and Measurement

VSV-pseudotyped third-generation lentiviral vectors (LVs) were produced by transient four-plasmid cotransfection into HEK293T cells and purified by anion-exchange as described in OXB patent (U.S. Pat. No. 9,169,491 B2). Vector particles were initially analyzed by functional titer and HIV-I gag p24 antigen immunocapture (NEN Life Science Products) to ensure proper transfection, production, and purification yields. Concentrated vector expression titer, or functional titer ranged from 1-10E8 TU/mL transducing units^{293T} (TU)/ml for all vectors.

### Cell Cultures

Functional titer was measured by transducing adherent HEK293T cells with lentivector. Cells were split three times and then harvested for genomic DNA isolation. LV integration was measured in the genomic DNA by droplet digital PCR (ddPCR) using lentivector specific primers and probes. HEK293T adherent cells were maintained in Iscove's modified Dulbecco's medium (IMDM; Gibco) supplemented with 10% fetal bovine serum (FBS; Gibco) and a combination of penicillin-streptomycin and glutamine.

Processing LVs into Vehicle (Formulation)

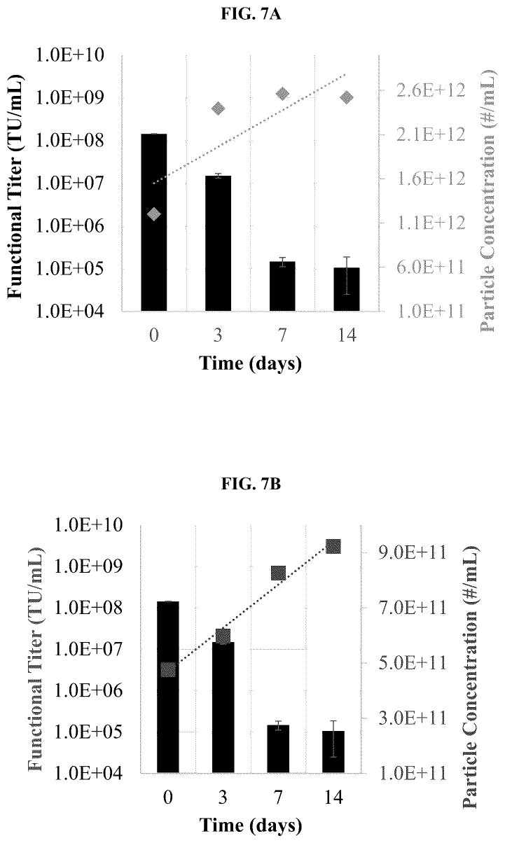

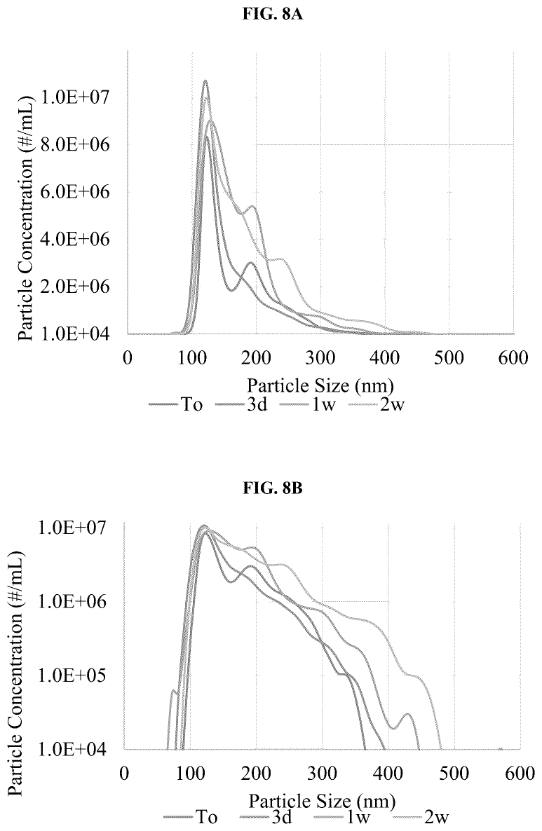

After purification the LV material (drug substance—DS) was pooled and buffer exchanged into respective formulation buffers using a hollow fiber membrane. The DS was first concentrated roughly ten-fold and then exchanged with the respective formulation buffer six times the volume of the concentrated DS (e.g. 6 mL buffer for every 1 mL of concentrated DS). The final formulated LV was considered Drug Product (DP) and was tested for stability. FIG. 7A-7B and FIGS. 8A-8B show characterization of the formulation upon re-processing into the vehicle Phosphate (Formulation 1) (10 mM phosphate, 100 mM NaCl, 3% (w/v) sucrose, 0.05% (w/v) P188, pH 7.3) from the vehicle TSSM (20 mM Tris, 100 mM NaCl, 1% (w/v) sucrose, 1% (w/v) mannitol, pH 7.3). In addition to TSSM, there were four alternative formulations tested herein: Formulation 2 (Phosphate HigherSalt). 10 mM Phosphate, 130 mM NaCl, 1% (w/v) sucrose, 0.05% (w/v) P188, pH 7.3; Formulation 3 (Histidine). 20 mM Histidine, 100 mM NaCl, 3% (w/v) sucrose, 0.05% (w/v) P188, pH 6.5; Formulation 4 (Phosphate pH 7.0). 10 mM Phosphate, 100 mM NaCl, 3% (w/v) sucrose, 0.05% (w/v) P188, pH 7.0; Formulation 5 (Histidine pH 7.0). 20 mM Histidine, 100 mM NaCl, 3% (w/v) sucrose, 0.05% (w/v) P188, pH 7.0.

### Example 2—In Vivo Administration of Lentiviral Vector (LV) Preparation

5-week old CD-1 and C57BL6 mice were purchased from Charles Rivers Laboratories and maintained in specific-pathogen-free conditions. 6 male HemA mice were obtained from our colony housed at Charles River. Administration of vector plus vehicle or vehicle alone was carried out by tail vein injection or temporal vein injection in mice. All animal procedures were performed according to protocols approved by Bioverativ/Sanofi IACUC (Animal Protocol 547). Three different formulations were tested: (1) 20 mM Tris, 100 mM NaCl, 1% (w/v) sucrose, 1% (w/v) mannitol, pH 7.3 (TSSM); (2) 10 mM phosphate, 100 mM NaCl, 3% (w/v) sucrose, 0.05% (w/v) P188, pH 7.3 (Phosphate); and (3) 20 mM histidine, 100 mM NaCl, 3% (w/v) sucrose, 0.05% (w/v) P188, pH 6.5 (Histidine) (Table 4).

TABLE 4
In vivo administration of LV preparations

| Formu- | | | | No |
|---|---|---|---|---|
| lation | Treatment | Fatality | Observation | Response |
| TSSM | Vector + Vehicle | x | x [rough coat, | |
| | | (n = 1) | excessive grooming, | |
| | | | hunched posture] | |
| | | | (n = 5) | |
| TSSM | Vehicle alone | | x [rough coat, | |
| | | | excessive grooming, | |
| | | | hunched posture] | |
| | | | (n = 4) | |
| Phosphate | Vector + Vehicle | | | x (n = 3) |
| Phosphate | Vehicle alone | | | x (n = 3) |
| Histidine | Vector + Vehicle | | | x (n = 3) |
| Histidine | Vehicle alone | | | x (n = 3) |

Formulation: Vehicle (TSSM) Alone and Vehicle (TSSM) with LV

A dose response study was conducted using LV-coFIX in C57BL6 and CD-1 adult mice after a single intravenous administration. 11 male C57BL6 mice (5 weeks old) and 11 male CD-1 mice (5 weeks old) were used. The doses administered were as follows: 6E10 (n=3), 2E10 (n=4), and 7.5E9 (n=4) TU/kg. The formulation vehicle was TSSM buffer.

C57BL6 and CD-1 mice were dosed with LV-FIX vector formulated in TSSM Buffer. The 6E10 TU/kg dose was given straight, no dilution at 13.3-15 ml/kg. The other two lower doses were diluted in PBS prior to administration. Three mice of each strain were given the high dose. Immediately upon injection of the 6E10 TU/kg dose, one C57BL6 mouse went into cardiac arrest and died. The other mice in the group exhibited adverse effects approximately a half hour later. We observed excessive grooming, then rough coat, listlessness, and inactivity. These mice seemed to recover after an hour post injection. Only the mice in the high dose (6E10 TU/kg) group showed an adverse effect. The mice that received a diluted dose showed no adverse effects at all. Later, 2 CD-1 and 2 C57BL6 mice were given just TSSM vehicle formulation buffer alone at 10-15 ml/kg. These mice also exhibited adverse effects post injection (excessive grooming, then rough coat, listlessness, and inactivity) that normalized after about an hour. The results are summarized in Table 4.

### Formulation: Vehicle (Phosphate) Alone

A dose response study was conducted using HemA mice. 3 male HemA mice (9 weeks old) were used. The dose administered was 15 ml/kg. The formulation vehicle was Phosphate buffer.

Three HemA mice were dosed with 15 ml/kg Phosphate formulation buffer to test for any adverse in-vivo effects. Mice were closely observed for the next 2 days. No adverse effects were noted including the excessive grooming, rough coat, listlessness, and inactivity seen with the TSSM formulation buffer.

Formulation: Vehicle (Phosphate) with LV

A dose response study was conducted using LV-coFVIII-6XTEN in HemA mice pups by temporal vein injection. 22 male and female HemA pups (2 days old) were used. The doses administered were as follows: 3E9 and 1.5E9 TU/kg. The formulation vehicle was Phosphate buffer.

Two day old mice were administered LV-FVIIIXTEN formulated in Phosphate Buffer at 3E9 or 1.5E9 TU/kg by temporal vein injection. The high dose (3E9) was given with no dilution. No adverse effects were seen in these mice post injection.

### Formulation: Vehicle (Histidine) Alone

A dose response study was conducted using HemA mice. 3 male HemA mice (19 weeks old) were used. The dose administered was 15 ml/kg. The formulation vehicle was Histidine buffer.

Three HemA mice were dosed with 15 ml/kg Histidine formulation buffer to test for any adverse in-vivo effects. Mice were closely observed for the next 2 days. No adverse effects were noted including the excessive grooming, rough coat, listlessness, and inactivity seen with the TSSM formulation buffer.

Formulation: Vehicle (Histidine) with LV

A dose response study was conducted using LV-coFVIII-6XTEN in tolerized adult HemA mice by tail vein injection. 4 male HF8 mice (12 weeks old) were used. The dose administered was 15 ml/kg. The formulation vehicle was Histidine buffer.

Four tolerized HemA mice (HF8) were dosed with 15 ml/kg LV-FVIIIXTEN vector formulated in Histidine formulation buffer. No adverse effects were noted including the excessive grooming, rough coat, listlessness, and inactivity seen with the TSSM formulation.

In summary, mice were injected with vector plus vehicle or vehicle alone for each of the formulations. Table 4 shows that TSSM injection, both vector plus vehicle and vehicle alone, had adverse toxic effects on mice, including one fatality. Conversely, Phosphate and Histidine formulations, vector plus vehicle or vehicle alone, resulted in no response (no toxic effects) in the mice.

### Example 3—Testing of Formulation Using the Vehicle TSSM

### Agitation, Freeze-Thaw, and Temperature Conditions

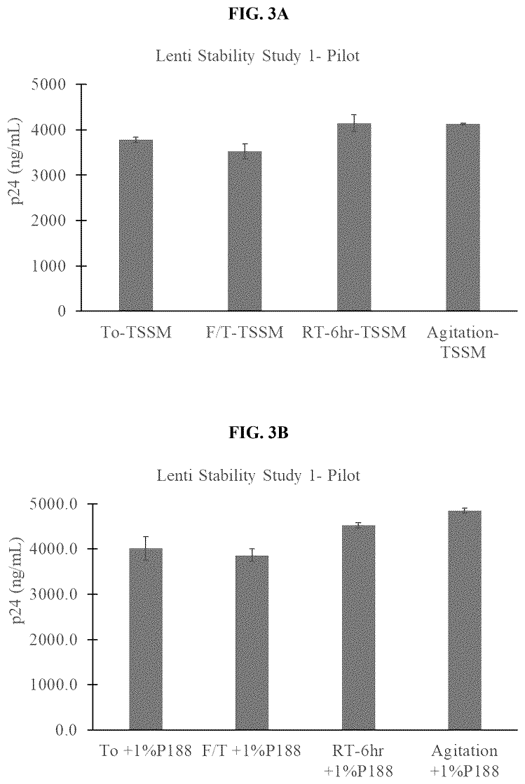

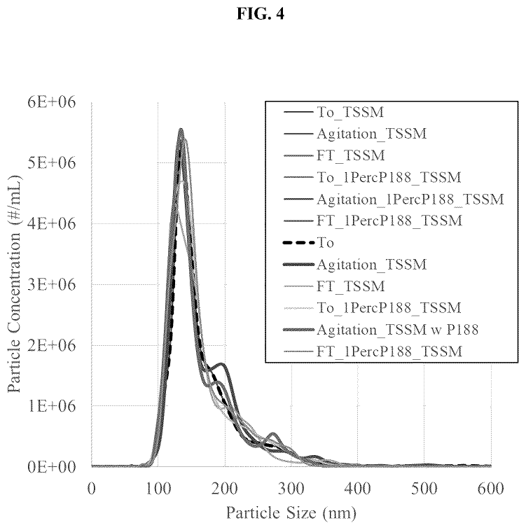

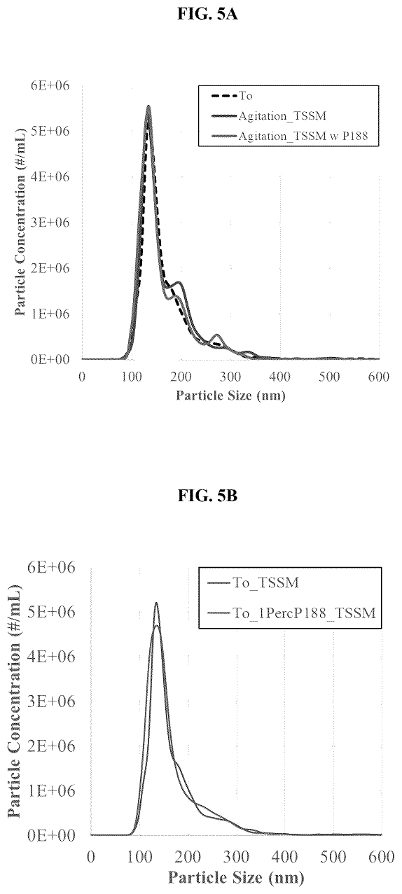

The stability of the TSSM formulation (vector plus vehicle) with and without the addition of 1% (w/v) P188, for a final concentration of 1% (w/v) P188, was tested by subjecting the formulation to agitation, freeze-thaw (F/T) cycles (5 and 10), and 6 hour room temperature incubation (FIGS. 3A-3B, FIG. 4, FIGS. 5A-5B, Table 5). Stability of the vector was measured by determining the functional titer, p24 concentration, and particle size and distribution (NanoSight). As shown in FIGS. 3A-3B, FIG. 4, FIGS. 5A-5B, and Table 5, there was no significant change in vector stability under the different conditions for the formulation with or without P188. Vector integrity was determined by particle size measurements using NanoSight.

TABLE 5
Testing of Formulation Using the Vehicle TSSM

| | | ddPCR | NanoSight | p24 | NanoSight/ddPCR | p24/ddPCR |
|---|---|---|---|---|---|---|
| Formulation | Stress | TU/mL | particles/mL | particles/mL | Inf. Ratio | Inf. Ratio |
| TSSM | To | 1.33E+08 | 6.08E+11 | 9.46E+11 | 4571 | 7109 |
| TSSM + | To | 1.27E+08 | 6.74E+11 | 1.00E+12 | 5292 | 7876 |
| P188 | | | | | | |
| TSSM | Agitation | 1.45E+08 | 6.50E+11 | 1.03E+12 | 4471 | 7101 |
| TSSM + | Agitation | 1.45E+08 | 6.46E+11 | 1.21E+12 | 4443 | 8339 |
| P188 | | | | | | |
| TSSM | RT-6 h | 1.29E+08 | NT | 1.04E+12 | NT | 8047 |
| TSSM + | RT-6 h | 1.34E+08 | NT | 1.13E+12 | NT | 8461 |
| P188 | | | | | | |
| TSSM | F/T | 1.49E+08 | 6.18E+11 | 8.81E+11 | 4153 | 5919 |
| TSSM + | F/T | 1.72E+08 | 6.78E+11 | 9.66E+11 | 3945 | 5620 |
| P188 | | | | | | |

*assuming 1 ng/mL p24 = 1.25E7 particles/mL (NT = Not Tested)

### Dilution Conditions

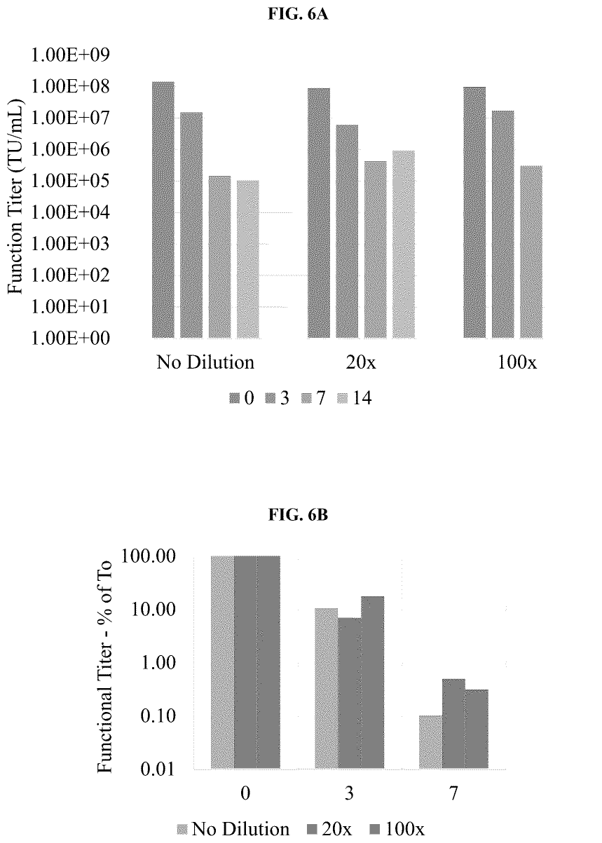

The TSSM formulation (vector plus vehicle) was diluted 1×, 20×, and 100×, incubated at 37° C., and stability determined through measurement of functional titer via ddPCR on days 0, 3, 7, and 14. FIG. 6A and FIG. 6B show that dilution had no effect on stability over two weeks at elevated temperature.

### Incubation for Different Time Periods

The TSSM formulation (vector plus vehicle) was incubated at 37° C. for 0, 3, 7, or 14 days. Stability was measured through determination of functional titer via ddPCR, while particle integrity (particle concentration) was measured using Nanosight or p24 ELISA. Stability and vector integrity (particle concentration) as a function of incubation time were determined, as shown in FIGS. 7A-7B and FIGS. 8A-8B. Vector stability decreased as incubation time increased, while particle concentration increased.

### Extended Incubation at 37° C.

The TSSM formulation (vector plus vehicle) was incubated at 37° C. for 0 days, 3 days, 1 week, or 2 weeks. Particle size distribution was measured using Nanosight. As shown in FIGS. 7A-7B and FIGS. 8A-8B, with extended incubation time, particle size distribution broadened, while particle concentration increased. The results may be explained as follows. The total particle concentration is increasing while infectivity is decreasing, because prolonged exposure to 37° C. temperature is causing the capsid to break apart, resulting in more apparent p24 measured in ELISA. Furthermore, the virus breaking apart leads to an increase in smaller sized species.

### Example 4—Testing Formulations Using the Vehicles Phosphate and Histidine Phosphate Formulation

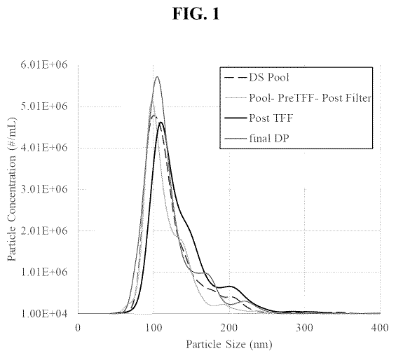

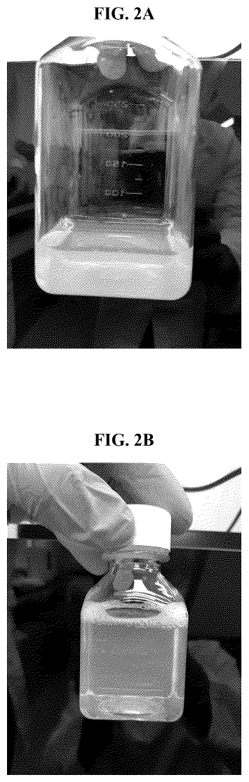

The lentiviral vector (LV) formulation was characterized upon processing into the vehicle Phosphate (10 mM Phosphate, 100 mM NaCl, 3% (w/v) sucrose, 0.05% (w/v) P188, pH 7.3). Particle size and distribution was measured using Nanosight (FIG. 1). FIG. 1 is a plot showing results from Nanosight showing a clean monomeric peak for the drug substance (DS) pool, which after ultrafiltration/diafiltration into the final vehicle buffer (post tangential flow filtration—TFF) shifts slightly to a larger particle size with the appearance of the presence of some larger particles. Without being bound to theory, this may be due to physical degradation of the particle during the stress of processing the material. The final drug product (DP) when filtered through a 0.22 μm sized filter membrane results in a particle size and distribution profile that returns back in line with the DS pool at the start of processing. TFF stress is visually shown in the photographs in FIGS. 2A-2B.

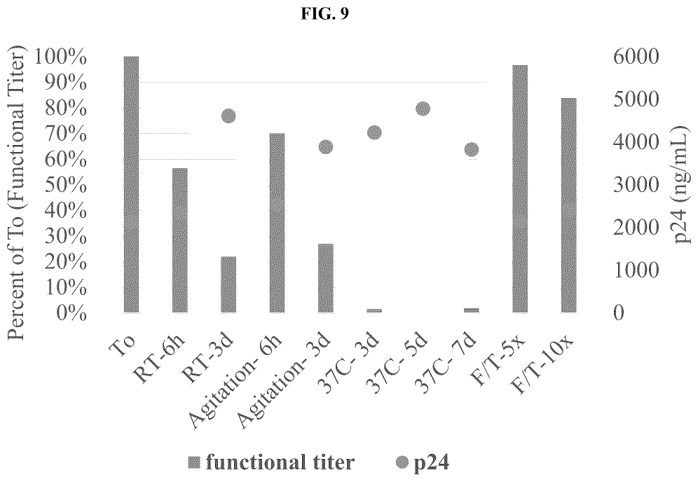

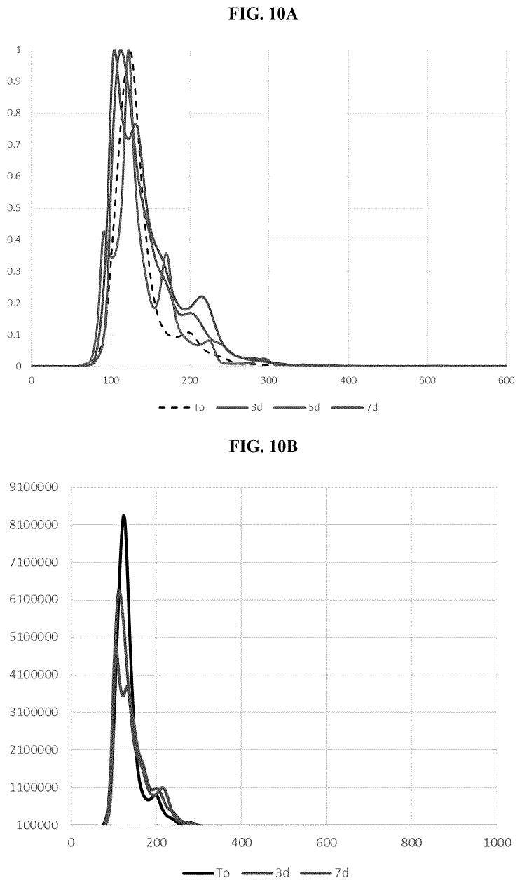

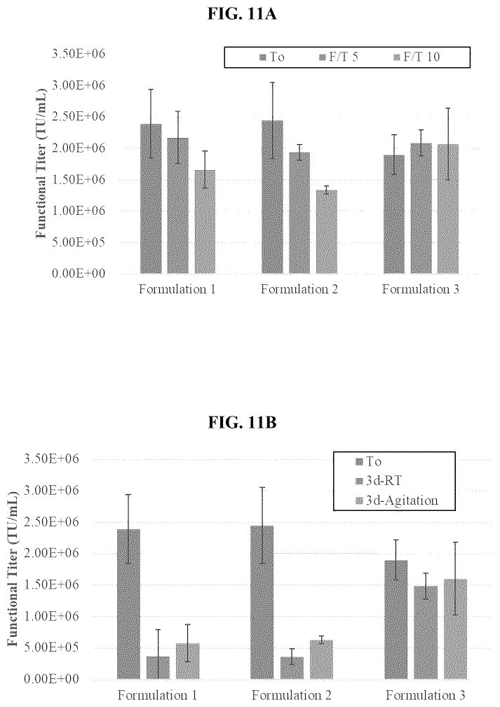

Vector stability was tested for the phosphate formulation (vector plus vehicle) under different conditions, including agitation, incubation at 37° C., duration, and dilution (FIG. 9 and FIGS. 10A-10B).

FIGS. 10A-10B shows the NanoSight size data for the phosphate buffer over one week at 37° C. There is a slight increase in higher molecular weight species as well as an overall drop in total particles over incubation time. The data suggests the loss in function titer shown in FIG. 9 over the course of stability at RT or 37° C. may correspond to the physical loss of particles observed in FIGS. 10A-10B.

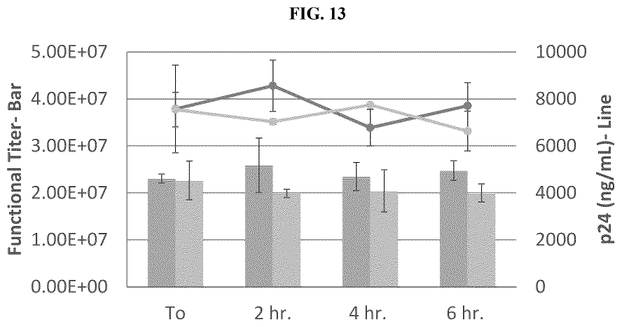

A mock in-use study was performed in order to assess the stability of the phosphate formulation over the course of a typical infusion scenario during clinical administration. The LVV material was diluted with the phosphate vehicle and injected into an empty IV bag. Data was collected over six hours at room temperature, FIG. 13. The stability study shows no signs of loss by functional titer or p24 indicating the vector was stable over the duration of the study.

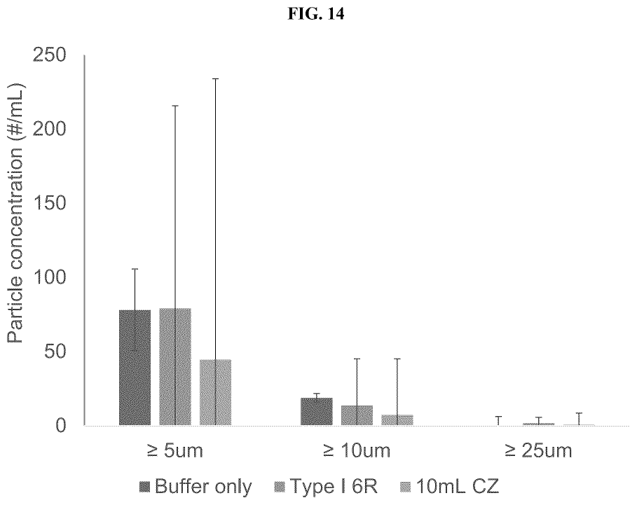

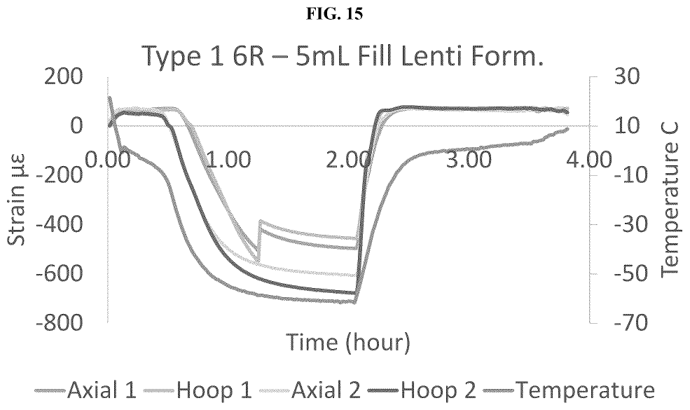

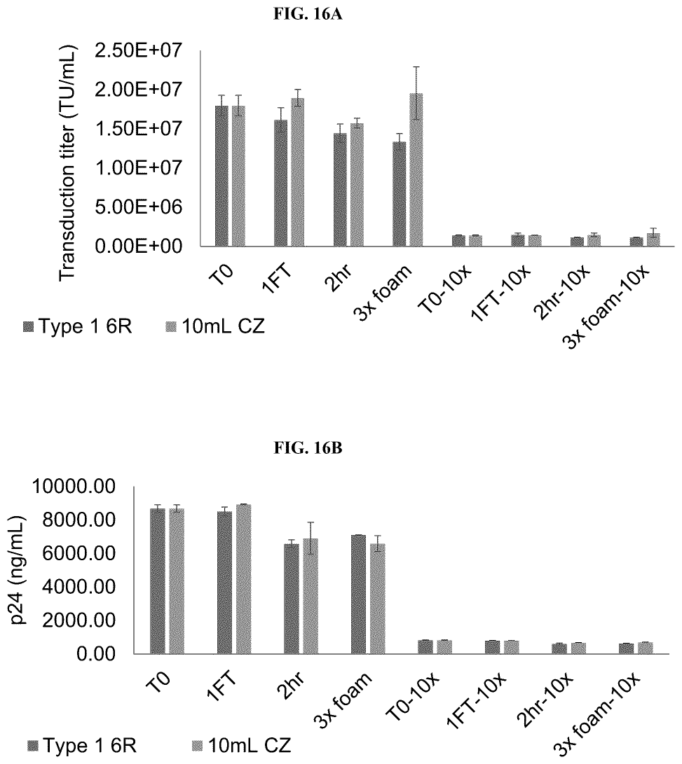

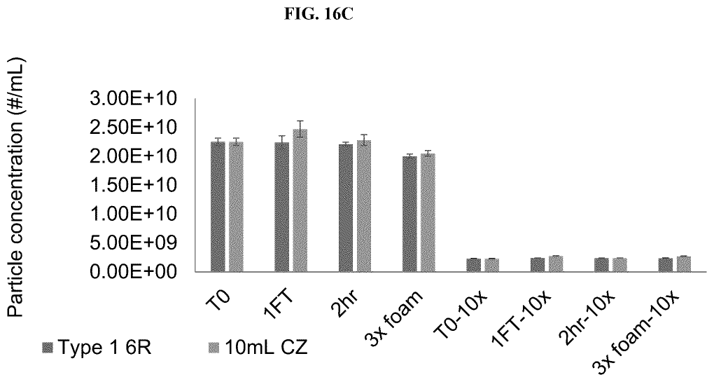

In order to test compatibility with a prospective container closure system, a study was performed in order to examine the stability of the vector and phosphate vehicle in Schott Type 1 glass vials as well as West CZ COP vials (FIG. 14, FIG. 15, and FIGS. 16A-16C). The data indicates that there were no significant particles produced when treated with the vehicle only in either the Type 1 or CZ vial, FIG. 14. In order to assess strain on the vial during freezing and thawing, a strain gauge was glued to the glass vial and placed in a −80° C. freezer for a period of two hours, followed by rapid warming in a 37° C. water bath, FIG. 15. The study indicates no appreciable positive strain on the vial, which would suggest that at this relatively high fill volume of 5 mL, the phosphate formulation does not impose high stress on the container, which has been shown in literature to damage certain vials. LVV material was also tested for compatibility with the respective containers, shown in FIGS. 16A-16C. Through each test, transduction titer (FIG. 16A), p24 (FIG. 16B), and particle concentration (FIG. 16C), there was comparability stability and integrity of the vector indicating compatibility with both container closure formats, glass and plastic vials.

### Phosphate and Histidine Formulation Comparisons

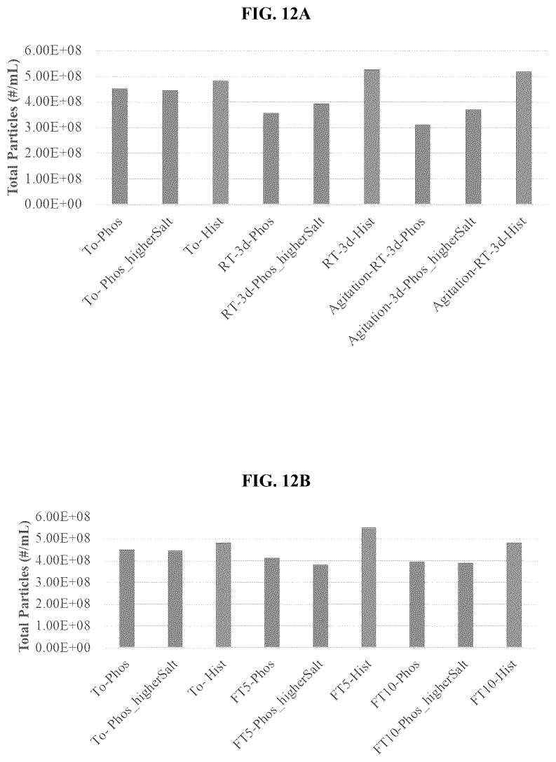

Vector stability was tested for the phosphate (Formulation 1 and Formulation 2) and histidine (Formulation 3) formulations (vector plus vehicle) under different stress conditions (FIGS. 11A-11B, FIGS. 12A-12B). In the freeze-thaw cycling study both phosphate formulations were observed to decrease in functional titer as a function of cycling. By contrast, the histidine formulation did not show a functional loss and remained stable. During the room temperature hold as well as the agitation stress condition, the phosphate formulation showed a high level of functional loss (down to the limit of quantitation). Surprisingly, the Histidine formulation remained unaffected by the incubation at room temperature for 3 days or the agitation for 3 days. Additionally, the addition of more sodium chloride and the drop in sucrose for the phosphate formulation (comparing Formulation 1 and Formulation 2) did not affect vector stability.

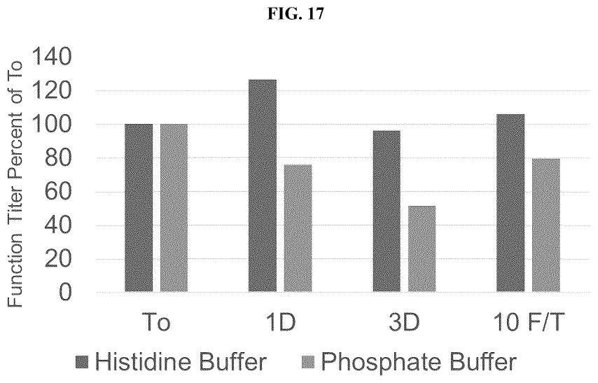

In a separate preparation of material, re-testing of the stability study demonstrated in FIGS. 11A-11B was repeated and reported in FIG. 17. Losses in functional titer were not as drastic for the phosphate formulation, but the trends are consistent between histidine and phosphate formulations.

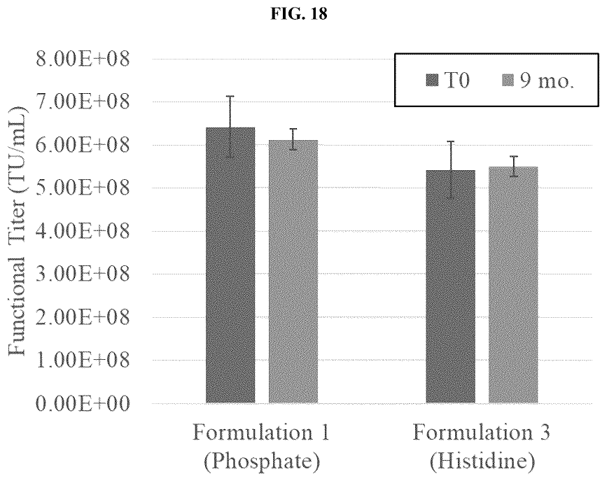

Over the course of a 9 month, frozen (−80° C.), stability study both the Phosphate Formulation 1 and the Histidine Formulation 3 buffers seemed to enable stable LVV DP, FIG. 18. Based on functional titer, the data suggests there was not any loss of material over storage at −80° C., within assay variability.

Lastly, in order to evaluate the effect of the buffers only (phosphate and histidine), the two formulations were prepared identically, at the same pH, 7.0. That is, the only difference between Formulation 4 and Formulation 5 was the buffer component (either phosphate or histidine). In the context of the manufacturing process, both formulations seem to behave similarly across ultrafiltration and diafiltration (TFF) as well as terminal sterile filtration, Table 6.

TABLE 6
Preparation of Formulation 4 (Phosphate) and Formulation 5
(Histidine) using Tangential Flow Filtration. The samples were
diafiltered against respective formulations six times by volume
and then ultrafiltered (concentrated) several fold, by volume.

| | | Volume | NanoSight | Total |
|---|---|---|---|---|
| Formulation | Process Step | (mL) | (particles/mL) | Particles |
| Phosphate | Pre TFF | 165.2 | 1.28E+11 | 2.11E+13 |
| Phosphate | Post 0.2 um filtration | 88.5 | 2.11E+11 | 1.87E+13 |
| Histidine | Pre TFF | 155.8 | 9.64E+10 | 1.50E+13 |
| Histidine | Post 0.2 um filtration | 65.2 | 2.54E+11 | 1.66E+13 |

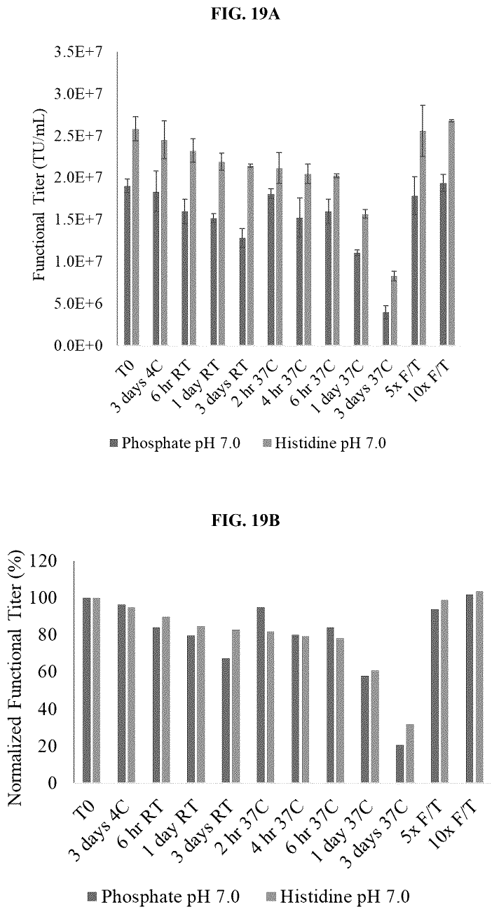

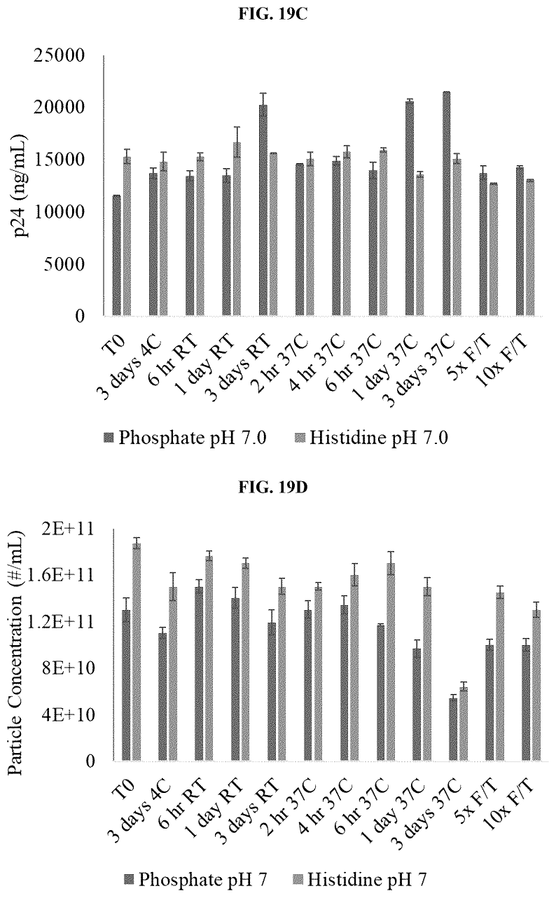

Upon preparing the respective formulations, a stability study was conducted in order to compare the buffer components. FIGS. 19A-19D summarizes the findings, shown as a function of stability condition across functional and normalized functional titer, p24 and particle concentration that there were only minor differences between the LVV stability of either formulation. This seems to suggest that results presented in FIGS. 11A-11B, FIGS. 12A-12B, and FIG. 13 seem to reflect varying levels of stability due to the differences in pH (7.3 and 6.5) and not the buffer composition (phosphate or histidine).

1-48. (canceled)

49. A reduced in-vivo toxicity recombinant lentiviral particle preparation comprising:
(a) a therapeutically effective dose of a lentiviral particle comprising a recombinant lentiviral vector;
(b) a TRIS-free buffer system comprising a phosphate buffer;
(c) a salt;
(d) a poloxamer 188 (P188);
(e) a carbohydrate;
wherein the lentiviral particle further comprises an envelope comprising a vesicular stomatitis virus G (VSV-G) protein or a fragment thereof;
wherein the recombinant lentiviral vector comprises a nucleic acid comprising a nucleotide sequence that is at least 80% identical to a Factor IX (FIX) coding sequence set forth in SEQ ID NO: 3;
wherein the recombinant lentiviral particle preparation is suitable for systemic administration to a human patient; and
wherein systemic administration of the recombinant lentiviral particle preparation to the patient has reduced in-vivo toxicity relative to a recombinant lentiviral particle preparation comprising a TRIS buffer.

50. The preparation of claim 49, wherein the pH of the buffer system or of the preparation is from about from about 7.0 to about 8.0.

51. The preparation of claim 49, wherein the concentration of the phosphate buffer is from about 5 mM to about 30 mM.

52. The preparation of claim 49, wherein the concentration of the phosphate buffer is from about 10 mM to about 20 mM.

53. The preparation of claim 49, wherein the concentration of the salt is from about 80 mM to about 150 mM.

54. The preparation of claim 49, wherein the concentration of the poloxamer 188 is from about 0.01% (w/v) to about 0.1% (w/v), about 0.03% (w/v), about 0.05% (w/v), about 0.07% (w/v), or about 0.09% (w/v).

55. The preparation of claim 49, wherein the concentration of the carbohydrate is from about 0.50% (w/v) to about 50% (w/v), about 1% (w/v), about 2% (w/v), about 30% (w/v), or about 4% (w/v), optionally wherein the carbohydrate is sucrose.

56. The preparation of claim 49, comprising:
(a) a therapeutically effective dose of a recombinant lentiviral vector;
(b) about 10 mM phosphate buffer;
(c) about 100 mM sodium chloride;
(d) about 0.05% (w/v) P188; and
(e) about 3% (w/v) sucrose,
wherein the pH of the preparation is about 7.3, and wherein the preparation is suitable for systemic administration to a human patient.

57. The preparation of claim 49, comprising:
(a) a therapeutically effective dose of a recombinant lentiviral vector;
(b) about 10 mM phosphate buffer;
(c) about 130 mM sodium chloride;
(d) about 0.05% (w/v) P188; and
(e) about 1% (w/v) sucrose,
wherein the pH of the preparation is about 7.3, and wherein the preparation is suitable for systemic administration to a human patient.

58. The preparation of claim 49, comprising:
(a) a therapeutically effective dose of a recombinant lentiviral vector;
(b) about 10 mM phosphate buffer;
(c) about 100 mM sodium chloride;
(d) about 0.05% (w/v) P188; and
(e) about 3% (w/v) sucrose,
wherein the pH of the preparation is about 7.0, and wherein the preparation is suitable for systemic administration to a human patient.

59. The preparation of claim 49, wherein the recombinant lentiviral vector comprises:
an enhanced transthyretin (ET) promoter; and/or
a nucleotide sequence at least 90% identical to the target sequence for miR-142 set forth in SEQ ID NO: 7.

60. The preparation of claim 49, wherein the recombinant lentiviral vector is isolated from a transfected host cell selected from the group consisting of a CHO cell, a HEK293 cell, a BHK21 cell, a PER.C6 cell, an NSO cell, and a CAP cell.

61. The preparation of claim 49, wherein the recombinant lentiviral vector comprises a nucleic acid comprising the FIX coding sequence set forth in SEQ ID NO: 3.

62. A reduced in-vivo toxicity recombinant lentiviral particle preparation, comprising:
(a) a therapeutically effective dose of a lentiviral particle comprising a recombinant lentiviral vector;
(b) a TRIS-free buffer system comprising about 10 mM phosphate buffer;
(c) about 100 mM or about 130 mM sodium chloride;
(d) about 0.05% (w/v) P188;
(e) about 3% (w/v) sucrose;
wherein the pH of the preparation is about 7.3;
wherein the lentiviral particle preparation is suitable for systemic administration to a human patient;
wherein the recombinant lentiviral particle further comprises an envelope comprising a vesicular stomatitis virus G (VSV-G) protein or a fragment thereof;
wherein the recombinant lentiviral vector comprises a nucleic acid comprising a nucleotide sequence that is at least 80% identical to a Factor IX (FIX) coding sequence set forth in SEQ ID NO: 3; and
wherein administration of the lentiviral particle preparation to a patient has reduced in-vivo toxicity relative to a recombinant lentiviral particle preparation comprising a TRIS buffer.

63. A reduced in-vivo toxicity recombinant lentiviral particle preparation, comprising:
(a) a therapeutically effective dose of a lentiviral particle comprising a recombinant lentiviral vector;
(b) a TRIS-free buffer system comprising about 10 mM phosphate buffer;
(c) about 100 mM or about 130 mM sodium chloride;
(d) about 0.05% (w/v) P188;
(e) about 3% (w/v) sucrose;
wherein the pH of the preparation is about 7.0; and wherein the lentiviral particle preparation is suitable for systemic administration to a human patient;
wherein the recombinant lentiviral particle further comprises an envelope comprising a vesicular stomatitis virus G (VSV-G) protein or a fragment thereof;
wherein the recombinant lentiviral vector comprises a nucleic acid comprising a nucleotide sequence that is at least 80% identical to a Factor IX (FIX) coding sequence set forth in SEQ ID NO: 3; and
wherein administration of the lentiviral particle preparation to a patient has reduced in-vivo toxicity relative to a recombinant lentiviral particle preparation comprising a TRIS buffer.


SEQUENCE LISTING (63 sequences)

SEQ ID NO: 1 - DNA, 4371 bp, synthetic construct
  misc_feature 1..4371: note = Synthetic polynucleotide sequence
  source 1..4371: mol_type = other DNA
atgcagattgagctgtccacttgtttcttcctgtgcctcctgcgcttctgtttctccgccactcgccggtactaccttggagccgtggagctttcatgggactacatgcagagcgacctgggcgaactccccgtggatgccagattccccccccgcgtgccaaagtccttcccctttaacacctccgtggtgtacaagaaaaccctctttgtcgagttcactgaccacctgttcaacatcgccaagccgcgcccaccttggatgggcctcctgggaccgaccattcaagctgaagtgtacgacaccgtggtgatcaccctgaagaacatggcgtcccaccccgtgtccctgcatgcggtcggagtgtcctactggaaggcctccgaaggagctgagtacgacgaccagactagccagcgggaaaaggaggacgataaagtgttcccgggcggctcgcatacttacgtgtggcaagtcctgaaggaaaacggacctatggcatccgatcctctgtgcctgacttactcctacctttcccatgtggacctcgtgaaggacctgaacagcgggctgattggtgcacttctcgtgtgccgcgaaggttcgctcgctaaggaaaagacccagaccctccataagttcatccttttgttcgctgtgttcgatgaaggaaagtcatggcattccgaaactaagaactcgctgatgcaggaccgggatgccgcctcagcccgcgcctggcctaaaatgcatacagtcaacggatacgtgaatcggtcactgcccgggctcatcggttgtcacagaaagtccgtgtactggcacgtcatcggcatgggcactacgcctgaagtgcactccatcttcctggaagggcacaccttcctcgtgcgcaaccaccgccaggcctctctggaaatctccccgattacctttctgaccgcccagactctgctcatggacctggggcagttccttctcttctgccacatctccagccatcagcacgacggaatggaggcctacgtgaaggtggactcatgcccggaagaacctcagttgcggatgaagaacaacgaggaggccgaggactatgacgacgatttgactgactccgagatggacgtcgtgcggttcgatgacgacaacagccccagcttcatccagattcgcagcgtggccaagaagcaccccaaaacctgggtgcactacatcgcggccgaggaagaagattgggactacgccccgttggtgctggcacccgatgaccggtcgtacaagtcccagtatctgaacaatggtccgcagcggattggcagaaagtacaagaaagtgcggttcatggcgtacactgacgaaacgtttaagacccgggaggccattcaacatgagagcggcattctgggaccactgctgtacggagaggtcggcgataccctgctcatcatcttcaaaaaccaggcctcccggccttacaacatctaccctcacggaatcaccgacgtgcggccactctactcgcggcgcctgccgaagggcgtcaagcacctgaaagacttccctatcctgccgggcgaaatcttcaagtataagtggaccgtcaccgtggaggacgggcccaccaagagcgatcctaggtgtctgactcggtactactccagcttcgtgaacatggaacgggacctggcatcgggactcattggaccgctgctgatctgctacaaagagtcggtggatcaacgcggcaaccagatcatgtccgacaagcgcaacgtgatcctgttctccgtgtttgatgaaaacagatcctggtacctcactgaaaacatccagaggttcctcccaaaccccgcaggagtgcaactggaggaccctgagtttcaggcctcgaatatcatgcactcgattaacggttacgtgttcgactcgctgcagctgagcgtgtgcctccatgaagtcgcttactggtacattctgtccatcggcgcccagactgacttcctgagcgtgttcttttccggttacacctttaagcacaagatggtgtacgaagataccctgaccctgttccctttctccggcgaaacggtgttcatgtcgatggagaacccgggtctgtggattctgggatgccacaacagcgactttcggaaccgcggaatgactgccctgctgaaggtgtcctcatgcgacaagaacaccggagactactacgaggactcctacgaggatatctcagcctacctcctgtccaagaacaacgcgatcgagccgcgcagcttcagccagaacccgcctgtgctgaagaggcaccagcgagaaattacccggaccaccctccaatcggatcaggaggaaatcgactacgacgacaccatctcggtggaaatgaagaaggaagatttcgatatctacgacgaggacgaaaatcagtcccctcgctcattccaaaagaaaactagacactactttatcgccgcggtggaaagactgtgggactatggaatgtcatccagccctcacgtccttcggaaccgggcccagagcggatcggtgcctcagttcaagaaagtggtgttccaggagttcaccgacggcagcttcacccagccgctgtaccggggagaactgaacgaacacctgggcctgctcggtccctacatccgcgcggaagtggaggataacatcatggtgaccttccgtaaccaagcatccagaccttactccttctattcctccctgatctcatacgaggaggaccagcgccaaggcgccgagccccgcaagaacttcgtcaagcccaacgagactaagacctacttctggaaggtccaacaccatatggccccgaccaaggatgagtttgactgcaaggcctgggcctacttctccgacgtggaccttgagaaggatgtccattccggcctgatcgggccgctgctcgtgtgtcacaccaacaccctgaacccagcgcatggacgccaggtcaccgtccaggagtttgctctgttcttcaccatttttgacgaaactaagtcctggtacttcaccgagaatatggagcgaaactgtagagcgccctgcaatatccagatggaagatccgactttcaaggagaactatagattccacgccatcaacgggtacatcatggatactctgccggggctggtcatggcccaggatcagaggattcggtggtacttgctgtcaatgggatcgaacgaaaacattcactccattcacttctccggtcacgtgttcactgtgcgcaagaaggaggagtacaagatggcgctgtacaatctgtaccccggggtgttcgaaactgtggagatgctgccgtccaaggccggcatctggagagtggagtgcctgatcggagagcacctccacgcggggatgtccaccctcttcctggtgtactcgaataagtgccagaccccgctgggcatggcctcgggccacatcagagacttccagatcacagcaagcggacaatacggccaatgggcgccgaagctggcccgcttgcactactccggatcgatcaacgcatggtccaccaaggaaccgttctcgtggattaaggtggacctcctggcccctatgattatccacggaattaagacccagggcgccaggcagaagttctcctccctgtacatctcgcaattcatcatcatgtacagcctggacgggaagaagtggcagacttacaggggaaactccaccggcaccctgatggtctttttcggcaacgtggattcctccggcattaagcacaacatcttcaacccaccgatcatagccagatatattaggctccaccccactcactactcaatccgctcaactcttcggatggaactcatggggtgcgacctgaactcctgctccatgccgttggggatggaatcaaaggctattagcgacgcccagatcaccgcgagctcctacttcactaacatgttcgccacctggagcccctccaaggccaggctgcacttgcagggacggtcaaatgcctggcggccgcaagtgaacaatccgaaggaatggcttcaagtggatttccaaaagaccatgaaagtgaccggagtcaccacccagggagtgaagtcccttctgacctcgatgtatgtgaaggagttcctgattagcagcagccaggacgggcaccagtggaccctgttcttccaaaacggaaaggtcaaggtgttccaggggaaccaggactcgttcacacccgtggtgaactccctggaccccccactgctgacgcggtacttgaggattcatcctcagtcctgggtccatcagattgcattgcgaatggaagtcctgggctgcgaggcccaggacctgtac

SEQ ID NO: 2 - DNA, 4812 bp, synthetic construct
  misc_feature 1..4812: note = Synthetic polynucleotide sequence
  source 1..4812: mol_type = other DNA
atgcagattgagctgtccacttgtttcttcctgtgcctcctgcgcttctgtttctccgccactcgccggtactaccttggagccgtggagctttcatgggactacatgcagagcgacctgggcgaactccccgtggatgccagattccccccccgcgtgccaaagtccttcccctttaacacctccgtggtgtacaagaaaaccctctttgtcgagttcactgaccacctgttcaacatcgccaagccgcgcccaccttggatgggcctcctgggaccgaccattcaagctgaagtgtacgacaccgtggtgatcaccctgaagaacatggcgtcccaccccgtgtccctgcatgcggtcggagtgtcctactggaaggcctccgaaggagctgagtacgacgaccagactagccagcgggaaaaggaggacgataaagtgttcccgggcggctcgcatacttacgtgtggcaagtcctgaaggaaaacggacctatggcatccgatcctctgtgcctgacttactcctacctttcccatgtggacctcgtgaaggacctgaacagcgggctgattggtgcacttctcgtgtgccgcgaaggttcgctcgctaaggaaaagacccagaccctccataagttcatccttttgttcgctgtgttcgatgaaggaaagtcatggcattccgaaactaagaactcgctgatgcaggaccgggatgccgcctcagcccgcgcctggcctaaaatgcatacagtcaacggatacgtgaatcggtcactgcccgggctcatcggttgtcacagaaagtccgtgtactggcacgtcatcggcatgggcactacgcctgaagtgcactccatcttcctggaagggcacaccttcctcgtgcgcaaccaccgccaggcctctctggaaatctccccgattacctttctgaccgcccagactctgctcatggacctggggcagttccttctcttctgccacatctccagccatcagcacgacggaatggaggcctacgtgaaggtggactcatgcccggaagaacctcagttgcggatgaagaacaacgaggaggccgaggactatgacgacgatttgactgactccgagatggacgtcgtgcggttcgatgacgacaacagccccagcttcatccagattcgcagcgtggccaagaagcaccccaaaacctgggtgcactacatcgcggccgaggaagaagattgggactacgccccgttggtgctggcacccgatgaccggtcgtacaagtcccagtatctgaacaatggtccgcagcggattggcagaaagtacaagaaagtgcggttcatggcgtacactgacgaaacgtttaagacccgggaggccattcaacatgagagcggcattctgggaccactgctgtacggagaggtcggcgataccctgctcatcatcttcaaaaaccaggcctcccggccttacaacatctaccctcacggaatcaccgacgtgcggccactctactcgcggcgcctgccgaagggcgtcaagcacctgaaagacttccctatcctgccgggcgaaatcttcaagtataagtggaccgtcaccgtggaggacgggcccaccaagagcgatcctaggtgtctgactcggtactactccagcttcgtgaacatggaacgggacctggcatcgggactcattggaccgctgctgatctgctacaaagagtcggtggatcaacgcggcaaccagatcatgtccgacaagcgcaacgtgatcctgttctccgtgtttgatgaaaacagatcctggtacctcactgaaaacatccagaggttcctcccaaaccccgcaggagtgcaactggaggaccctgagtttcaggcctcgaatatcatgcactcgattaacggttacgtgttcgactcgctgcagctgagcgtgtgcctccatgaagtcgcttactggtacattctgtccatcggcgcccagactgacttcctgagcgtgttcttttccggttacacctttaagcacaagatggtgtacgaagataccctgaccctgttccctttctccggcgaaacggtgttcatgtcgatggagaacccgggtctgtggattctgggatgccacaacagcgactttcggaaccgcggaatgactgccctgctgaaggtgtcctcatgcgacaagaacaccggagactactacgaggactcctacgaggatatctcagcctacctcctgtccaagaacaacgcgatcgagccgcgcagcttcagccagaacacatcagagagcgccacccctgaaagtggtcccgggagcgagccagccacatctgggtcggaaacgccaggcacaagtgagtctgcaactcccgagtccggacctggctccgagcctgccactagcggctccgagactccgggaacttccgagagcgctacaccagaaagcggacccggaaccagtaccgaacctagcgagggctctgctccgggcagcccagccggctctcctacatccacggaggagggcacttccgaatccgccaccccggagtcagggccaggatctgaacccgctacctcaggcagtgagacgccaggaacgagcgagtccgctacaccggagagtgggccagggagccctgctggatctcctacgtccactgaggaagggtcaccagcgggctcgcccaccagcactgaagaaggtgcctcgagcccgcctgtgctgaagaggcaccagcgagaaattacccggaccaccctccaatcggatcaggaggaaatcgactacgacgacaccatctcggtggaaatgaagaaggaagatttcgatatctacgacgaggacgaaaatcagtcccctcgctcattccaaaagaaaactagacactactttatcgccgcggtggaaagactgtgggactatggaatgtcatccagccctcacgtccttcggaaccgggcccagagcggatcggtgcctcagttcaagaaagtggtgttccaggagttcaccgacggcagcttcacccagccgctgtaccggggagaactgaacgaacacctgggcctgctcggtccctacatccgcgcggaagtggaggataacatcatggtgaccttccgtaaccaagcatccagaccttactccttctattcctccctgatctcatacgaggaggaccagcgccaaggcgccgagccccgcaagaacttcgtcaagcccaacgagactaagacctacttctggaaggtccaacaccatatggccccgaccaaggatgagtttgactgcaaggcctgggcctacttctccgacgtggaccttgagaaggatgtccattccggcctgatcgggccgctgctcgtgtgtcacaccaacaccctgaacccagcgcatggacgccaggtcaccgtccaggagtttgctctgttcttcaccatttttgacgaaactaagtcctggtacttcaccgagaatatggagcgaaactgtagagcgccctgcaatatccagatggaagatccgactttcaaggagaactatagattccacgccatcaacgggtacatcatggatactctgccggggctggtcatggcccaggatcagaggattcggtggtacttgctgtcaatgggatcgaacgaaaacattcactccattcacttctccggtcacgtgttcactgtgcgcaagaaggaggagtacaagatggcgctgtacaatctgtaccccggggtgttcgaaactgtggagatgctgccgtccaaggccggcatctggagagtggagtgcctgatcggagagcacctccacgcggggatgtccaccctcttcctggtgtactcgaataagtgccagaccccgctgggcatggcctcgggccacatcagagacttccagatcacagcaagcggacaatacggccaatgggcgccgaagctggcccgcttgcactactccggatcgatcaacgcatggtccaccaaggaaccgttctcgtggattaaggtggacctcctggcccctatgattatccacggaattaagacccagggcgccaggcagaagttctcctccctgtacatctcgcaattcatcatcatgtacagcctggacgggaagaagtggcagacttacaggggaaactccaccggcaccctgatggtctttttcggcaacgtggattcctccggcattaagcacaacatcttcaacccaccgatcatagccagatatattaggctccaccccactcactactcaatccgctcaactcttcggatggaactcatggggtgcgacctgaactcctgctccatgccgttggggatggaatcaaaggctattagcgacgcccagatcaccgcgagctcctacttcactaacatgttcgccacctggagcccctccaaggccaggctgcacttgcagggacggtcaaatgcctggcggccgcaagtgaacaatccgaaggaatggcttcaagtggatttccaaaagaccatgaaagtgaccggagtcaccacccagggagtgaagtcccttctgacctcgatgtatgtgaaggagttcctgattagcagcagccaggacgggcaccagtggaccctgttcttccaaaacggaaaggtcaaggtgttccaggggaaccaggactcgttcacacccgtggtgaactccctggaccccccactgctgacgcggtacttgaggattcatcctcagtcctgggtccatcagattgcattgcgaatggaagtcctgggctgcgaggcccaggacctgtac

SEQ ID NO: 3 - DNA, 1383 bp, synthetic construct
  misc_feature 1..1383: note = Synthetic polynucleotide sequence
  source 1..1383: mol_type = other DNA
atgcagagagtcaacatgattatggctgagtcacctgggctgattactatttgcctgctgggctacctgctgtccgccgagtgtaccgtgttcctggaccatgagaacgcaaataagatcctgaacaggcccaaaagatacaatagtgggaagctggaggaatttgtgcagggcaacctggagagagaatgcatggaggaaaagtgtagcttcgaggaagcccgcgaggtgtttgaaaatacagagcgaaccacagagttctggaagcagtatgtggacggcgatcagtgcgagagcaacccctgtctgaatggcggaagttgcaaagacgatatcaactcatacgaatgctggtgtcctttcgggtttgaaggcaaaaattgcgagctggacgtgacatgtaacattaagaatggacggtgcgagcagttttgtaaaaactctgccgataataaggtggtgtgcagctgtactgaaggatatcgcctggctgagaaccagaagtcctgcgaaccagcagtgcccttcccttgtgggagggtgagcgtctcccagacttcaaaactgaccagagcagagacagtgtttcccgacgtggattacgtcaacagcactgaggccgaaaccatcctggacaacattactcagtctacccagagtttcaatgactttactcgggtggtcgggggcgaggatgctaaaccaggccagttcccctggcaggtggtcctgaacggaaaggtggatgcattttgcggagggtctatcgtgaatgagaaatggattgtcaccgccgctcactgcgtggaaaccggagtcaagatcacagtggtcgctggggagcacaacattgaggaaacagaacatactgagcagaagcggaatgtgatccgcatcattcctcaccataactacaatgcagccatcaacaaatacaatcatgacattgccctgctggaactggatgagcctctggtgctgaacagctacgtcactccaatctgcattgctgacaaagagtataccaatatcttcctgaagtttggatcagggtacgtgagcggctggggaagagtcttccacaagggcaggagcgccctggtgctccagtatctgcgagtgcctctggtcgatcgagctacctgtctgctctctaccaagtttacaatctacaacaacatgttctgcgctgggtttcacgagggaggacgagactcctgtcagggcgattctgggggcccacatgtgacagaggtcgaaggcaccagcttcctgactggcatcatttcctggggagaggaatgtgcaatgaagggaaaatacgggatctacaccaaagtgagccgctatgtgaactggatcaaggaaaaaaccaaactgacc

SEQ ID NO: 4 - protein, 2332 aa, synthetic construct
  REGION 1..2332: note = Synthetic amino acid sequence
ATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNSRHPSTRQKQFNATTIPENDIEKTDPWFAHRTPMPKIQNVSSSDLLMLLRQSPTPHGLSLSDLQEAKYETFSDDPSPGAIDSNNSLSEMTHFRPQLHHSGDMVFTPESGLQLRLNEKLGTTAATELKKLDFKVSSTSNNLISTIPSDNLAAGTDNTSSLGPPSMPVHYDSQLDTTLFGKKSSPLTESGGPLSLSEENNDSKLLESGLMNSQESSWGKNVSSTESGRLFKGKRAHGPALLTKDNALFKVSISLLKTNKTSNNSATNRKTHIDGPSLLIENSPSVWQNILESDTEFKKVTPLIHDRMLMDKNATALRLNHMSNKTTSSKNMEMVQQKKEGPIPPDAQNPDMSFFKMLFLPESARWIQRTHGKNSLNSGQGPSPKQLVSLGPEKSVEGQNFLSEKNKVVVGKGEFTKDVGLKEMVFPSSRNLFLTNLDNLHENNTHNQEKKIQEEIEKKETLIQENVVLPQIHTVTGTKNFMKNLFLLSTRQNVEGSYDGAYAPVLQDFRSLNDSTNRTKKHTAHFSKKGEEENLEGLGNQTKQIVEKYACTTRISPNTSQQNFVTQRSKRALKQFRLPLEETELEKRIIVDDTSTQWSKNMKHLTPSTLTQIDYNEKEKGAITQSPLSDCLTRSHSIPQANRSPLPIAKVSSFPSIRPIYLTRVLFQDNSSHLPAASYRKKDSGVQESSHFLQGAKKNNLSLAILTLEMTGDQREVGSLGTSATNSVTYKKVENTVLPKPDLPKTSGKVELLPKVHIYQKDLFPTETSNGSPGHLDLVEGSLLQGTEGAIKWNEANRPGKVPFLRVATESSAKTPSKLLDPLAWDNHYGTQIPKEEWKSQEKSPEKTAFKKKDTILSLNACESNHAIAAINEGQNKPEIEVTWAKQGRTERLCSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 5 - protein, 1604 aa, synthetic construct
  REGION 1..1604: note = Synthetic amino acid sequence
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGASSPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 6 - protein, 461 aa, synthetic construct
  REGION 1..461: note = Synthetic amino acid sequence
MQRVNMIMAESPGLITICLLGYLLSAECTVFLDHENANKILNRPKRYNSGKLEEFVQGNLERECMEEKCSFEEAREVFENTERTTEFWKQYVDGDQCESNPCLNGGSCKDDINSYECWCPFGFEGKNCELDVTCNIKNGRCEQFCKNSADNKVVCSCTEGYRLAENQKSCEPAVPFPCGRVSVSQTSKLTRAETVFPDVDYVNSTEAETILDNITQSTQSFNDFTRVVGGEDAKPGQFPWQVVLNGKVDAFCGGSIVNEKWIVTAAHCVETGVKITVVAGEHNIEETEHTEQKRNVIRIIPHHNYNAAINKYNHDIALLELDEPLVLNSYVTPICIADKEYTNIFLKFGSGYVSGWGRVFHKGRSALVLQYLRVPLVDRATCLLSTKFTIYNNMFCAGFHEGGRDSCQGDSGGPHVTEVEGTSFLTGIISWGEECAMKGKYGIYTKVSRYVNWIKEKTKLT

SEQ ID NO: 7 - DNA, 23 bp, synthetic construct
  misc_feature 1..23: note = Synthetic polynucleotide sequence
  source 1..23: mol_type = other DNA
tccataaagtaggaaacactaca

SEQ ID NO: 8 - protein, 4 aa, synthetic construct
  REGION 1..4: note = Synthetic amino acid sequence
ELLG

SEQ ID NO: 9 - protein, 32 aa, synthetic construct
  REGION 1..32: note = Synthetic amino acid sequence
DPRFQDSSSSKAPPPSLPSPSRLPGPSDTPIL

SEQ ID NO: 10 - protein, 28 aa, synthetic construct
  REGION 1..28: note = Synthetic amino acid sequence
SSSSKAPPPSLPSPSRLPGPSDTPILPQ

SEQ ID NO: 11 - protein, 12 aa, synthetic construct
  REGION 1..12: note = Synthetic amino acid sequence
GESPGGSSGSES

SEQ ID NO: 12 - protein, 12 aa, synthetic construct
  REGION 1..12: note = Synthetic amino acid sequence
GSEGSSGPGESS

SEQ ID NO: 13 - protein, 12 aa, synthetic construct
  REGION 1..12: note = Synthetic amino acid sequence
GSSESGSSEGGP

SEQ ID NO: 14 - protein, 12 aa, synthetic construct
  REGION 1..12: note = Synthetic amino acid sequence
GSGGEPSESGSS

SEQ ID NO: 15 - protein, 12 aa, synthetic construct
  REGION 1..12: note = Synthetic amino acid sequence
GSPAGSPTSTEE

SEQ ID NO: 16 - protein, 12 aa, synthetic construct
  REGION 1..12: note = Synthetic amino acid sequence
GSEPATSGSETP

SEQ ID NO: 17 - protein, 12 aa, synthetic construct
  REGION 1..12: note = Synthetic amino acid sequence
GTSESATPESGP

SEQ ID NO: 18 - protein, 12 aa, synthetic construct
  REGION 1..12: note = Synthetic amino acid sequence
GTSTEPSEGSAP

SEQ ID NO: 19 - protein, 12 aa, synthetic construct
  REGION 1..12: note = Synthetic amino acid sequence
GSTSESPSGTAP

SEQ ID NO: 20 - protein, 12 aa, synthetic construct
  REGION 1..12: note = Synthetic amino acid sequence
GTSTPESGSASP

SEQ ID NO: 21 - protein, 12 aa, synthetic construct
  REGION 1..12: note = Synthetic amino acid sequence
GTSPSGESSTAP

SEQ ID NO: 22 - protein, 12 aa, synthetic construct
  REGION 1..12: note = Synthetic amino acid sequence
GSTSSTAESPGP

SEQ ID NO: 23 - protein, 12 aa, synthetic construct
  REGION 1..12: note = Synthetic amino acid sequence
GTPGSGTASSSP

SEQ ID NO: 24 - protein, 12 aa, synthetic construct
  REGION 1..12: note = Synthetic amino acid sequence
GSSTPSGATGSP

SEQ ID NO: 25 - protein, 12 aa, synthetic construct
  REGION 1..12: note = Synthetic amino acid sequence
GSSPSASTGTGP

SEQ ID NO: 26 - protein, 12 aa, synthetic construct
  REGION 1..12: note = Synthetic amino acid sequence
GASPGTSSTGSP

SEQ ID NO: 27 - protein, 12 aa, synthetic construct
  REGION 1..12: note = Synthetic amino acid sequence
GEPAGSPTSTSE

SEQ ID NO: 28 - protein, 12 aa, synthetic construct
  REGION 1..12: note = Synthetic amino acid sequence
GTGEPSSTPASE

SEQ ID NO: 29 - protein, 12 aa, synthetic construct
  REGION 1..12: note = Synthetic amino acid sequence
GSGPSTESAPTE

SEQ ID NO: 30 - protein, 12 aa, synthetic construct
  REGION 1..12: note = Synthetic amino acid sequence
GSETPSGPSETA

SEQ ID NO: 31 - protein, 12 aa, synthetic construct
  REGION 1..12: note = Synthetic amino acid sequence
GPSETSTSEPGA

SEQ ID NO: 32 - protein, 12 aa, synthetic construct
  REGION 1..12: note = Synthetic amino acid sequence
GSPSEPTEGTSA

SEQ ID NO: 33 - protein, 12 aa, synthetic construct
  REGION 1..12: note = Synthetic amino acid sequence
GSGASEPTSTEP

SEQ ID NO: 34 - protein, 12 aa, synthetic construct
  REGION 1..12: note = Synthetic amino acid sequence
GSEPATSGTEPS

SEQ ID NO: 35 - protein, 12 aa, synthetic construct
  REGION 1..12: note = Synthetic amino acid sequence
GTSEPSTSEPGA

SEQ ID NO: 36 - protein, 12 aa, synthetic construct
  REGION 1..12: note = Synthetic amino acid sequence
GTSTEPSEPGSA

SEQ ID NO: 37 - protein, 12 aa, synthetic construct
  REGION 1..12: note = Synthetic amino acid sequence
GSTAGSETSTEA

SEQ ID NO: 38 - protein, 12 aa, synthetic construct
  REGION 1..12: note = Synthetic amino acid sequence
GSETATSGSETA

SEQ ID NO: 39 - protein, 12 aa, synthetic construct
  REGION 1..12: note = Synthetic amino acid sequence
GTSESATSESGA

SEQ ID NO: 40 - protein, 12 aa, synthetic construct
  REGION 1..12: note = Synthetic amino acid sequence
GTSTEASEGSAS

SEQ ID NO: 41 - protein, 42 aa, synthetic construct
  REGION 1..42: note = Synthetic amino acid sequence
GAPGSPAGSPTSTEEGTSESATPESGPGSEPATSGSETPASS

SEQ ID NO: 42 - protein, 144 aa, synthetic construct
  REGION 1..144: note = Synthetic amino acid sequence
TSTEPSEGSAPGSPAGSPTSTEEGTSTEPSEGSAPGTSTEPSEGSAPGTSESATPESGPGTSTEPSEGSAPGTSESATPESGPGSEPATSGSETPGTSTEPSEGSAPGTSTEPSEGSAPGTSESATPESGPGTSESATPESGPG

SEQ ID NO: 43 - protein, 144 aa, synthetic construct
  REGION 1..144: note = Synthetic amino acid sequence
SPAGSPTSTEEGTSESATPESGPGSEPATSGSETPGTSESATPESGPGTSTEPSEGSAPGTSTEPSEGSAPGTSTEPSEGSAPGTSTEPSEGSAPGTSTEPSEGSAPGTSTEPSEGSAPGSPAGSPTSTEEGTSTEPSEGSAPG

SEQ ID NO: 44 - protein, 144 aa, synthetic construct
  REGION 1..144: note = Synthetic amino acid sequence
TSESATPESGPGSEPATSGSETPGTSESATPESGPGSEPATSGSETPGTSESATPESGPGTSTEPSEGSAPGTSESATPESGPGSPAGSPTSTEEGSPAGSPTSTEEGSPAGSPTSTEEGTSESATPESGPGTSTEPSEGSAPG

SEQ ID NO: 45 - protein, 144 aa, synthetic construct
  REGION 1..144: note = Synthetic amino acid sequence
TSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEG

SEQ ID NO: 46 - protein, 144 aa, synthetic construct
  REGION 1..144: note = Synthetic amino acid sequence
TSTEPSEGSAPGTSESATPESGPGTSESATPESGPGTSESATPESGPGSEPATSGSETPGSEPATSGSETPGSPAGSPTSTEEGTSTEPSEGSAPGTSTEPSEGSAPGSEPATSGSETPGTSESATPESGPGTSTEPSEGSAPG

SEQ ID NO: 47 - protein, 144 aa, synthetic construct
  REGION 1..144: note = Synthetic amino acid sequence
PGSSPSASTGTGPGSSPSASTGTGPGTPGSGTASSSPGSSTPSGATGSPGSSPSASTGTGPGASPGTSSTGSPGTPGSGTASSSPGSSTPSGATGSPGTPGSGTASSSPGASPGTSSTGSPGASPGTSSTGSPGTPGSGTASSS

SEQ ID NO: 48 - protein, 144 aa, synthetic construct
  REGION 1..144: note = Synthetic amino acid sequence
GASPGTSSTGSPGSSPSASTGTGPGSSPSASTGTGPGTPGSGTASSSPGSSTPSGATGSPGSSPSASTGTGPGASPGTSSTGSPGTPGSGTASSSPGSSTPSGATGSPGTPGSGTASSSPGASPGTSSTGSPGASPGTSSTGSP

SEQ ID NO: 49 - protein, 144 aa, synthetic construct
  REGION 1..144: note = Synthetic amino acid sequence
GTPGSGTASSSPGSSTPSGATGSPGASPGTSSTGSPGTPGSGTASSSPGSSTPSGATGSPGSSPSASTGTGPGSSPSASTGTGPGSSTPSGATGSPGSSTPSGATGSPGASPGTSSTGSPGASPGTSSTGSPGASPGTSSTGSP

SEQ ID NO: 50 - protein, 144 aa, synthetic construct
  REGION 1..144: note = Synthetic amino acid sequence
GTPGSGTASSSPGASPGTSSTGSPGASPGTSSTGSPGASPGTSSTGSPGSSPSASTGTGPGTPGSGTASSSPGASPGTSSTGSPGASPGTSSTGSPGASPGTSSTGSPGSSTPSGATGSPGSSTPSGATGSPGASPGTSSTGSP

SEQ ID NO: 51 - protein, 144 aa, synthetic construct
  REGION 1..144: note = Synthetic amino acid sequence
GSSPSASTGTGPGSSPSASTGTGPGASPGTSSTGSPGASPGTSSTGSPGSSTPSGATGSPGSSPSASTGTGPGASPGTSSTGSPGSSPSASTGTGPGTPGSGTASSSPGSSTPSGATGSPGSSTPSGATGSPGASPGTSSTGSP

SEQ ID NO: 52 - DNA, 450 bp, synthetic construct
  misc_feature 1..450: note = Synthetic polynucleotide sequence
  source 1..450: mol_type = other DNA
ggcgcgccaacatcagagagcgccacccctgaaagtggtcccgggagcgagccagccacatctgggtcggaaacgccaggcacaagtgagtctgcaactcccgagtccggacctggctccgagcctgccactagcggctccgagactccgggaacttccgagagcgctacaccagaaagcggacccggaaccagtaccgaacctagcgagggctctgctccgggcagcccagccggctctcctacatccacggaggagggcacttccgaatccgccaccccggagtcagggccaggatctgaacccgctacctcaggcagtgagacgccaggaacgagcgagtccgctacaccggagagtgggccagggagccctgctggatctcctacgtccactgaggaagggtcaccagcgggctcgcccaccagcactgaagaaggtgcctcgagc

SEQ ID NO: 53 - skipped

SEQ ID NO: 54 - protein, 11 aa, synthetic construct
  REGION 1..11: note = Synthetic amino acid sequence
DICLPRWGCLW

SEQ ID NO: 55 - protein, 20 aa, synthetic construct
  REGION 1..20: note = Synthetic amino acid sequence
ASPAAPAPASPAAPAPSAPA

SEQ ID NO: 56 - protein, 20 aa, synthetic construct
  REGION 1..20: note = Synthetic amino acid sequence
AAPASPAPAAPSAPAPAAPS

SEQ ID NO: 57 - protein, 20 aa, synthetic construct
  REGION 1..20: note = Synthetic amino acid sequence
APSSPSPSAPSSPSPASPSS

SEQ ID NO: 58 - protein, 19 aa, synthetic construct
  REGION 1..19: note = Synthetic amino acid sequence
APSSPSPSAPSSPSPASPS

SEQ ID NO: 59 - protein, 20 aa, synthetic construct
  REGION 1..20: note = Synthetic amino acid sequence
SSPSAPSPSSPASPSPSSPA

SEQ ID NO: 60 - protein, 24 aa, synthetic construct
  REGION 1..24: note = Synthetic amino acid sequence
AASPAAPSAPPAAASPAAPSAPPA

SEQ ID NO: 61 - protein, 20 aa, synthetic construct
  REGION 1..20: note = Synthetic amino acid sequence
ASAAAPAAASAAASAPSAAA

SEQ ID NO: 62 - protein, 2813 aa, synthetic construct
  REGION 1..2813: note = Synthetic amino acid sequence
MIPARFAGVLLALALILPGTLCAEGTRGRSSTARCSLFGSDFVNTFDGSMYSFAGYCSYLLAGGCQKRSFSIIGDFQNGKRVSLSVYLGEFFDIHLFVNGTVTQGDQRVSMPYASKGLYLETEAGYYKLSGEAYGFVARIDGSGNFQVLLSDRYFNKTCGLCGNFNIFAEDDFMTQEGTLTSDPYDFANSWALSSGEQWCERASPPSSSCNISSGEMQKGLWEQCQLLKSTSVFARCHPLVDPEPFVALCEKTLCECAGGLECACPALLEYARTCAQEGMVLYGWTDHSACSPVCPAGMEYRQCVSPCARTCQSLHINEMCQERCVDGCSCPEGQLLDEGLCVESTECPCVHSGKRYPPGTSLSRDCNTCICRNSQWICSNEECPGECLVTGQSHFKSFDNRYFTFSGICQYLLARDCQDHSFSIVIETVQCADDRDAVCTRSVTVRLPGLHNSLVKLKHGAGVAMDGQDVQLPLLKGDLRIQHTVTASVRLSYGEDLQMDWDGRGRLLVKLSPVYAGKTCGLCGNYNGNQGDDFLTPSGLAEPRVEDFGNAWKLHGDCQDLQKQHSDPCALNPRMTRFSEEACAVLTSPTFEACHRAVSPLPYLRNCRYDVCSCSDGRECLCGALASYAAACAGRGVRVAWREPGRCELNCPKGQVYLQCGTPCNLTCRSLSYPDEECNEACLEGCFCPPGLYMDERGDCVPKAQCPCYYDGEIFQPEDIFSDHHTMCYCEDGFMHCTMSGVPGSLLPDAVLSSPLSHRSKRSLSCRPPMVKLVCPADNLRAEGLECTKTCQNYDLECMSMGCVSGCLCPPGMVRHENRCVALERCPCFHQGKEYAPGETVKIGCNTCVCRDRKWNCTDHVCDATCSTIGMAHYLTFDGLKYLFPGECQYVLVQDYCGSNPGTFRILVGNKGCSHPSVKCKKRVTILVEGGEIELFDGEVNVKRPMKDETHFEVVESGRYIILLLGKALSVVWDRHLSISVVLKQTYQEKVCGLCGNFDGIQNNDLTSSNLQVEEDPVDFGNSWKVSSQCADTRKVPLDSSPATCHNNIMKQTMVDSSCRILTSDVFQDCNKLVDPEPYLDVCIYDTCSCESIGDCACFCDTIAAYAHVCAQHGKVVTWRTATLCPQSCEERNLRENGYECEWRYNSCAPACQVTCQHPEPLACPVQCVEGCHAHCPPGKILDELLQTCVDPEDCPVCEVAGRRFASGKKVTLNPSDPEHCQICHCDVVNLTCEACQEPGGLVVPPTDAPVSPTTLYVEDISEPPLHDFYCSRLLDLVFLLDGSSRLSEAEFEVLKAFVVDMMERLRISQKWVRVAVVEYHDGSHAYIGLKDRKRPSELRRIASQVKYAGSQVASTSEVLKYTLFQIFSKIDRPEASRITLLLMASQEPQRMSRNFVRYVQGLKKKKVIVIPVGIGPHANLKQIRLIEKQAPENKAFVLSSVDELEQQRDEIVSYLCDLAPEAPPPTLPPDMAQVTVGPGLLGVSTLGPKRNSMVLDVAFVLEGSDKIGEADFNRSKEFMEEVIQRMDVGQDSIHVTVLQYSYMVTVEYPFSEAQSKGDILQRVREIRYQGGNRTNTGLALRYLSDHSFLVSQGDREQAPNLVYMVTGNPASDEIKRLPGDIQVVPIGVGPNANVQELERIGWPNAPILIQDFETLPREAPDLVLQRCCSGEGLQIPTLSPAPDCSQPLDVILLLDGSSSFPASYFDEMKSFAKAFISKANIGPRLTQVSVLQYGSITTIDVPWNVVPEKAHLLSLVDVMQREGGPSQIGDALGFAVRYLTSEMHGARPGASKAVVILVTDVSVDSVDAAADAARSNRVTVFPIGIGDRYDAAQLRILAGPAGDSNVVKLQRIEDLPTMVTLGNSFLHKLCSGFVRICMDEDGNEKRPGDVWTLPDQCHTVTCQPDGQTLLKSHRVNCDRGLRPSCPNSQSPVKVEETCGCRWTCPCVCTGSSTRHIVTFDGQNFKLTGSCSYVLFQNKEQDLEVILHNGACSPGARQGCMKSIEVKHSALSVELHSDMEVTVNGRLVSVPYVGGNMEVNVYGAIMHEVRFNHLGHIFTFTPQNNEFQLQLSPKTFASKTYGLCGICDENGANDFMLRDGTVTTDWKTLVQEWTVQRPGQTCQPILEEQCLVPDSSHCQVLLLPLFAECHKVLAPATFYAICQQDSCHQEQVCEVIASYAHLCRTNGVCVDWRTPDFCAMSCPPSLVYNHCEHGCPRHCDGNVSSCGDHPSEGCFCPPDKVMLEGSCVPEEACTQCIGEDGVQHQFLEAWVPDHQPCQICTCLSGRKVNCTTQPCPTAKAPTCGLCEVARLRQNADQCCPEYECVCDPVSCDLPPVPHCERGLQPTLTNPGECRPNFTCACRKEECKRVSPPSCPPHRLPTLRKTQCCDEYECACNCVNSTVSCPLGYLASTATNDCGCTTTTCLPDKVCVHRSTIYPVGQFWEEGCDVCTCTDMEDAVMGLRVAQCSQKPCEDSCRSGFTYVLHEGECCGRCLPSACEVVTGSPRGDSQSSWKSVGSQWASPENPCLINECVRVKEEVFIQQRNVSCPQLEVPVCPSGFQLSCKTSACCPSCRCERMEACMLNGTVIGPGKTVMIDVCTTCRCMVQVGVISGFKLECRKTTCNPCPLGYKEENNTGECCGRCLPTACTIQLRGGQIMTLKRDETLQDGCDTHFCKVNERGEYFWEKRVTGCPPFDEHKCLAEGGKIMKIPGTCCDTCEEPECNDITARLQYVKVGSCKSEVEVDIHYCQGKCASKAMYSIDINDVQDQCSCCSPTRTEPMQVALHCTNGSVVYHEVLNAMECKCSPRKCSK

SEQ ID NO: 63 - DNA, 577 bp, synthetic construct
  misc_feature 1..577: note = Synthetic polynucleotide sequence
  source 1..577: mol_type = other DNA
ctcgaggtcaattcacgcgagttaataattaccagcgcgggccaaataaataatccgcgaggggcaggtgacgtttgcccagcgcgcgctggtaattattaacctcgcgaatattgattcgaggccgcgattgccgcaatcgcgaggggcaggtgacctttgcccagcgcgcgttcgccccgccccggacggtatcgataagcttaggagcttgggctgcaggtcgagggcactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgccaccatgg